SEQUENCE LISTING (26 sequences)

SEQ ID NO: 1 - DNA, 303 bp, Homo sapiens
aagcggcgagtccaaggggctaagggcttccagcaccaacggatgaccaacggggccatgaacgtggagattggaaaccccacctacaagatgtacgaaggcggagagcctgatgatgtgggaggcctactggacgctgactttgccctggaccctgacaagcccaccaacttcaccaaccccgtgtatgccacactctacatggggggccatggcagtcgccactccctggccagcacggacgagaagcgagaactcctgggccggggccctgaggacgagataggggaccccttggcatag

SEQ ID NO: 2 - DNA, 1179 bp, Homo sapiens
atgctgaccccgccgttgctcctgctgctgcccctgctctcagctctggtcgcggcggctatcgacaagcggctggacaacggcacatgcgtgcctgtgccctctccaacgccccccccagatgctccccggcctggaacctgtaacctgcagtgcttcaacggtggcagctgtttcctcaatgcacggaggcagcccaagtgccgctgccaaccccgctacacgggtgacaagtgtgaactggaccagtgctgggagcactgtcgcaatgggggcacctgtgctgcctccccctctggcatgcccacgtgccggtgccccacgggcttcacgggccccaaatgcacccagcaggtgtgtgcgggctactgtgccaacaacagcacctgcactgtcaaccagggcaaccagccccagtgccgatgcctacccggcttcctgggcgaccgctgccagtaccggcagtgctctggctactgtgagaactttggcacatgccagatggctgctgatggctcccgacaatgccgctgcactgcctactttgagggatcgaggtgtgaggtgaacaagtgcagccgctgtctcgaaggggcctgtgtggtcaacaagcagagtggggatgtcacctgcaactgcacggatggccgggtggcccccagctgtctgacctgcgtcggccactgcagcaatggcggctcctgtaccatgaacagcaaaatgatgcctgagtgccagtgcccaccccacatgacagggccccggtgtgaggagcacgtcttcagccagcagcagccaggacatatagcctccatcctaatccctctgctgttgctgctgctgctggttctggtggccggagtggtattctggtataagcggcgagtccaaggggctaagggcttccagcaccaacggatgaccaaacggggccatgaacgtggagattggaaacccacctacaagatgtacgaaggcggagagcctgatgatgtgggaggcctactggacgctgactttgccctggaccctgacaagcccaccaacttcaccaaccccgtgtatgccacactctacatggggggccatggcagtcgccactccctggccagcacggacgagaagcgagaactcctgggccggggccctgaggacgagataggggaccccttggcatag

SEQ ID NO: 3 - DNA, 303 bp, Homo sapiens
aagcggcgagtccaaggggctaagggcttccagcaccaacggatgaccaacggggccatgaacgtggagattggaaaccccacctacaagatgtacgaaggcggagagcctgatgatgtgggaggcctactggacgctgactttgccctggaccctgacaagcccaccaacttcaccgctcccgtgtatgccacactctacatggggggccatggcagtcgccactccctggccagcacggacgagaagcgagaactcctgggccggggccctgaggacgagataggggaccccttggcatag

SEQ ID NO: 4 - DNA, 303 bp, Homo sapiens
aagcggcgagtccaaggggctaagggcttccagcaccaacggatgaccaacggggccatgaacgtggagattggaaaccccacctacaagatgtacgaaggcggagagcctgatgatgtgggaggcctactggacgctgactttgccctggaccctgacaagcccaccaacttcaccaaccccgtggctgccacactctacatggggggccatggcagtcgccactccctggccagcacggacgagaagcgagaactcctgggccggggccctgaggacgagataggggaccccttggcatag

SEQ ID NO: 5 - DNA, 303 bp, Homo sapiens
aagcggcgagtccaaggggctaagggcttccagcaccaacggatgaccaacggggccatgaacgtggagattggaaaccccacctacaagatgtacgaaggcggagagcctgatgatgtgggaggcctactggacgctgactttgccctggaccctgacaagcccaccaacttcaccaaccccgtgtatgccacagcttacatggggggccatggcagtcgccactccctggccagcacggacgagaagcgagaactcctgggccggggccctgaggacgagataggggaccccttggcatag

SEQ ID NO: 6 - DNA, 1134 bp, Homo sapiens
atgctgaccccgccgttgctcctgctgctgcccctgctctcagctctggtcgcggcggctatcgacaagcggctggacaacggcacatgcgtgcctgtgccctctccaacgccccccccagatgctccccggcctggaacctgtaacctgcagtgcttcaacggtggcagctgtttcctcaatgcacggaggcagcccaagtgccgctgccaaccccgctacacgggtgacaagtgtgaactggaccagtgctgggagcactgtcgcaatgggggcacctgtgctgcctccccctctggcatgcccacgtgccggtgccccacgggcttcacgggccccaaatgcacccagcaggtgtgtgcgggctactgtgccaacaacagcacctgcactgtcaaccagggcaaccagccccagtgccgatgcctacccggcttcctgggcgaccgctgccagtaccggcagtgctctggctactgtgagaactttggcacatgccagatggctgctgatggctcccgacaatgccgctgcactgcctactttgagggatcgaggtgtgaggtgaacaagtgcagccgctgtctcgaaggggcctgtgtggtcaacaagcagagtggggatgtcacctgcaactgcacggatggccgggtggcccccagctgtctgacctgcgtcggccactgcagcaatggcggctcctgtaccatgaacagcaaaatgatgcctgagtgccagtgcccaccccacatgacagggccccggtgtgaggagcacgtcttcagccagcagcagccaggacatatagcctccatcctaatccctctgctgttgctgctgctgctggttctggtggccggagtggtattctggtataagcggcgagtccaaggggctaagggcttccagcaccaacggatgaccaacggggccatgaacgtggagattggaggaggcctactggacgctgactttgccctggaccctgacaagcccaccaacttcaccaaccccgtgtatgccacactctacatggggggccatggcagtcgccactccctggccagcacggacgagaagcgagaactcctgggccggggccctgaggacgagataggggaccccttggcatag

SEQ ID NO: 7 - DNA, 1116 bp, Homo sapiens
atgctgaccccgccgttgctcctgctgctgcccctgctctcagctctggtcgcggcggctatcgacaagcggctggacaacggcacatgcgtgcctgtgccctctccaacgccccccccagatgctccccggcctggaacctgtaacctgcagtgcttcaacggtggcagctgtttcctcaatgcacggaggcagcccaagtgccgctgccaaccccgctacacgggtgacaagtgtgaactggaccagtgctgggagcactgtcgcaatgggggcacctgtgctgcctccccctctggcatgcccacgtgccggtgccccacgggcttcacgggccccaaatgcacccagcaggtgtgtgcgggctactgtgccaacaacagcacctgcactgtcaaccagggcaaccagccccagtgccgatgcctacccggcttcctgggcgaccgctgccagtaccggcagtgctctggctactgtgagaactttggcacatgccagatggctgctgatggctcccgacaatgccgctgcactgcctactttgagggatcgaggtgtgaggtgaacaagtgcagccgctgtctcgaaggggcctgtgtggtcaacaagcagagtggggatgtcacctgcaactgcacggatggccgggtggcccccagctgtctgacctgcgtcggccactgcagcaatggcggctcctgtaccatgaacagcaaaatgatgcctgagtgccagtgcccaccccacatgacagggccccggtgtgaggagcacgtcttcagccagcagcagccaggacatatagcctccatcctaatccctctgctgttgctgctgctgctggttctggtggccggagtggtattctggtataagcggcgagtccaaggggctaagggcttccagcaccaacggatgaccaacggggccatgaacgtggagattggaaaccccacctacaagatgtacgaaggcggagagcctgatgatgtgggaggcgccacactctacatggggggccatggcagtcgccactccctggccagcacggacgagaagcgagaactcctgggccggggccctgaggacgagataggggaccccttggcatag

SEQ ID NO: 8 - DNA, 1101 bp, Homo sapiens
aagcggctggacaacggcacatgcgtgcctgtgccctctccaacgccccccccagatgctccccggcctggaacctgtaacctgcagtgcttcaacggtggcagctgtttcctcaatgcacggaggcagcccaagtgccgctgccaaccccgctacacgggtgacaagtgtgaactggaccagtgctgggagcactgtcgcaatgggggcacctgtgctgcctccccctctggcatgcccacgtgccggtgccccacgggcttcacgggccccaaatgcacccagcaggtgtgtgcgggctactgtgccaacaacagcacctgcactgtcaaccagggcaaccagccccagtgccgatgcctacccggcttcctgggcgaccgctgccagtaccggcagtgctctggctactgtgagaactttggcacatgccagatggctgctgatggctcccgacaatgccgctgcactgcctactttgagggatcgaggtgtgaggtgaacaagtgcagccgctgtctcgaaggggcctgtgtggtcaacaagcagagtggggatgtcacctgcaactgcacggatggccgggtggcccccagctgtctgacctgcgtcggccactgcagcaatggcggctcctgtaccatgaacagcaaaatgatgcctgagtgccagtgcccaccccacatgacagggccccggtgtgaggagcacgtcttcagccagcagcagccaggacatatagcctccatcctaatccctctgctgttgctgctgctgctggttctggtggccggagtggtattctggtataagcggcgagtccaaggggctaagggcttccagcaccaacggatgaccaacggggccatgaacgtggagattggaaaccccacctacaagatgtacgaaggcggagagcctgatgatgtgggaggcctactggacgctgactttgccctggaccctgacaagcccaccaacttcaccgccacactctacatggggggccatggcagtcgccactccctggccagcacggacgagaagcgagaactcctgggccggggccctgaggacgagataggggaccccttggcatag

SEQ ID NO: 9 - DNA, 1167 bp, Homo sapiens
atgctgaccccgccgttgctcctgctgctgcccctgctctcagctctggtcgcggcggctatcgacaagcggctggacaacggcacatgcgtgcctgtgccctctccaacgccccccccagatgctccccggcctggaacctgtaacctgcagtgcttcaacggtggcagctgtttcctcaatgcacggaggcagcccaagtgccgctgccaaccccgctacacgggtgacaagtgtgaactggaccagtgctgggagcactgtcgcaatgggggcacctgtgctgcctccccctctggcatgcccacgtgccggtgccccacgggcttcacgggccccaaatgcacccagcaggtgtgtgcgggctactgtgccaacaacagcacctgcactgtcaaccagggcaaccagccccagtgccgatgcctacccggcttcctgggcgaccgctgccagtaccggcagtgctctggctactgtgagaactttggcacatgccagatggctgctgatggctcccgacaatgccgctgcactgcctactttgagggatcgaggtgtgaggtgaacaagtgcagccgctgtctcgaaggggcctgtgtggtcaacaagcagagtggggatgtcacctgcaactgcacggatggccgggtggcccccagctgtctgacctgcgtcggccactgcagcaatggcggctcctgtaccatgaacagcaaaatgatgcctgagtgccagtgcccaccccacatgacagggccccggtgtgaggagcacgtcttcagccagcagcagccaggacatatagcctccatcctaatccctctgctgttgctgctgctgctggttctggtggccggagtggtattctggtataagcggcgagtccaaggggctaagggcttccagcaccaacggatgaccaacggggccatgaacgtggagattggaaaccccacctacaagatgtacgaaggcggagagcctgatgatgtgggaggcctactggacgctgactttgccctggaccctgacaagcccaccaacttcaccaaccccgtgtacatggggggccatggcagtcgccactccctggccagcacggacgagaagcgagaactcctgggccggggccctgaggacgagataggggaccccttggcatag

SEQ ID NO: 10 - DNA, 1179 bp, Homo sapiens
atgctgaccccgccgttgctcctgctgctgcccctgctctcagctctggtcgcggcggctatcgacaagcggctggacaacggcacatgcgtgcctgtgccctctccaacgccccccccagatgctccccggcctggaacctgtaacctgcagtgcttcaacggtggcagctgtttcctcaatgcacggaggcagcccaagtgccgctgccaaccccgctacacgggtgacaagtgtgaactggaccagtgctgggagcactgtcgcaatgggggcacctgtgctgcctccccctctggcatgcccacgtgccggtgccccacgggcttcacgggccccaaatgcacccagcaggtgtgtgcgggctactgtgccaacaacagcacctgcactgtcaaccagggcaaccagccccagtgccgatgcctacccggcttcctgggcgaccgctgccagtaccggcagtgctctggctactgtgagaactttggcacatgccagatggctgctgatggctcccgacaatgccgctgcactgcctactttgagggatcgaggtgtgaggtgaacaagtgcagccgctgtctcgaaggggcctgtgtggtcaacaagcagagtggggatgtcacctgcaactgcacggatggccgggtggcccccagctgtctgacctgcgtcggccactgcagcaatggcggctcctgtaccatgaacagcaaaatgatgcctgagtgccagtgcccaccccacatgacagggccccggtgtgaggagcacgtcttcagccagcagcagccaggacatatagcctccatcctaatccctctgctgttgctgctgctgctggttctggtggccggagtggtattctggtataagcggcgagtccaaggggctaagggcttccagcaccaacggatgaccaacggggccatgaacgtggagattggaaaccccacctacaagatgtacgaaggcggagagcctgatgatgtgggaggcctactggacgctgactttgccctggaccctgacaagcccaccaacttcaccgctcccgtgtatgccacactctacatggggggccatggcagtcgccactccctggccagcacggacgagaagcgagaactcctgggccggggccctgaggacgagataggggaccccttggcatag

SEQ ID NO: 11 - DNA, 1179 bp, Homo sapiens
atgctgaccccgccgttgctcctgctgctgcccctgctctcagctctggtcgcggcggctatcgacaagcggctggacaacggcacatgcgtgcctgtgccctctccaacgccccccccagatgctccccggcctggaacctgtaacctgcagtgcttcaacggtggcagctgtttcctcaatgcacggaggcagcccaagtgccgctgccaaccccgctacacgggtgacaagtgtgaactggaccagtgctgggagcactgtcgcaatgggggcacctgtgctgcctccccctctggcatgcccacgtgccggtgccccacgggcttcacgggccccaaatgcacccagcaggtgtgtgcgggctactgtgccaacaacagcacctgcactgtcaaccagggcaaccagccccagtgccgatgcctacccggcttcctgggcgaccgctgccagtaccggcagtgctctggctactgtgagaactttggcacatgccagatggctgctgatggctcccgacaatgccgctgcactgcctactttgagggatcgaggtgtgaggtgaacaagtgcagccgctgtctcgaaggggcctgtgtggtcaacaagcagagtggggatgtcacctgcaactgcacggatggccgggtggcccccagctgtctgacctgcgtcggccactgcagcaatggcggctcctgtaccatgaacagcaaaatgatgcctgagtgccagtgcccaccccacatgacagggccccggtgtgaggagcacgtcttcagccagcagcagccaggacatatagcctccatcctaatccctctgctgttgctgctgctgctggttctggtggccggagtggtattctggtataagcggcgagtccaaggggctaagggcttccagcaccaacggatgaccaacggggccatgaacgtggagattggaaaccccacctacaagatgtacgaaggcggagagcctgatgatgtgggaggcctactggacgctgactttgccctggaccctgacaagcccaccaacttcaccaaccccgtggctgccacactctacatggggggccatggcagtcgccactccctggccagcacggacgagaagcgagaactcctgggccggggccctgaggacgagataggggaccccttggcatag

SEQ ID NO: 12 - DNA, 1179 bp, Homo sapiens
atgctgaccccgccgttgctcctgctgctgcccctgctctcagctctggtcgcggcggctatcgacaagcggctggacaacggcacatgcgtgcctgtgccctctccaacgccccccccagatgctccccggcctggaacctgtaacctgcagtgcttcaacggtggcagctgtttcctcaatgcacggaggcagcccaagtgccgctgccaaccccgctacacgggtgacaagtgtgaactggaccagtgctgggagcactgtcgcaatgggggcacctgtgctgcctccccctctggcatgcccacgtgccggtgccccacgggcttcacgggccccaaatgcacccagcaggtgtgtgcgggctactgtgccaacaacagcacctgcactgtcaaccagggcaaccagccccagtgccgatgcctacccggcttcctgggcgaccgctgccagtaccggcagtgctctggctactgtgagaactttggcacatgccagatggctgctgatggctcccgacaatgccgctgcactgcctactttgagggatcgaggtgtgaggtgaacaagtgcagccgctgtctcgaaggggcctgtgtggtcaacaagcagagtggggatgtcacctgcaactgcacggatggccgggtggcccccagctgtctgacctgcgtcggccactgcagcaatggcggctcctgtaccatgaacagcaaaatgatgcctgagtgccagtgcccaccccacatgacagggccccggtgtgaggagcacgtcttcagccagcagcagccaggacatatagcctccatcctaatccctctgctgttgctgctgctgctggttctggtggccggagtggtattctggtataagcggcgagtccaaggggctaagggcttccagcaccaacggatgaccaacggggccatgaacgtggagattggaaaccccacctacaagatgtacgaaggcggagagcctgatgatgtgggaggcctactggacgctgactttgccctggaccctgacaagcccaccaacttcaccaaccccgtgtatgccacagcttacatggggggccatggcagtcgccactccctggccagcacggacgagaagcgagaactcctgggccggggccctgaggacgagataggggaccccttggcatag

SEQ ID NO: 13 - DNA, 13635 bp, Homo sapiens
atgctgaccccgccgttgctcctgctgctgcccctgctctcagctctggtcgcggcggctatcgacgcccctaagacttgcagccccaagcagtttgcctgcagagatcaaataacctgtatctcaaagggctggcggtgcgacggtgagagggactgcccagacggatctgacgaggcccctgagatttgtccacagagtaaggcccagcgatgccagccaaacgagcataactgcctgggtactgagctgtgtgttcccatgtcccgcctctgcaatggggtccaggactgcatggacggctcagatgaggggccccactgccgagagctccaaggcaactgctctcgcctgggctgccagcaccattgtgtccccacactcgatgggcccacctgctactgcaacagcagctttcagcttcaggcagatggcaagacctgcaaagattttgatgagtgctcagtgtacggcacctgcagccagctatgcaccaacacagacggctccttcatatgtggctgtgttgaaggatacctcctgcagccggataaccgctcctgcaaggccaagaacgagccagtagaccggccccctgtgctgttgatagccaactcccagaacatcttggccacgtacctgagtggggcccaggtgtctaccatcacacctacgagcacgcggcagaccacagccatggacttcagctatgccaacgagaccgtatgctgggtgcatgttggggacagtgctgctcagacgcagctcaagtgtgcccgcatgcctggcctaaagggcttcgtggatgagcacaccatcaacatctccctcagtctgcaccacgtggaacagatggccatcgactggctgacaggcaacttctactttgtggatgacatcgatgataggatctttgtctgcaacagaaatggggacacatgtgtcacattgctagacctggaactctacaaccccaagggcattgccctggaccctgccatggggaaggtgtttttcactgactatgggcagatcccaaaggtggaacgctgtgacatggatgggcagaaccgcaccaagctcgtcgacagcaagattgtgtttcctcatggcatcacgctggacctggtcagccgccttgtctactgggcagatgcctatctggactatattgaagtggtggactatgagggcaagggccgccagaccatcatccagggcatcctgattgagcacctgtacggcctgactgtgtttgagaattatctctatgccaccaactcggacaatgccaatgcccagcagaagacgagtgtgatccgtgtgaaccgctttaacagcaccgagtaccaggttgtcacccgggtggacaagggtggtgccctccacatctaccaccagaggcgtcagccccgagtgaggagccatgcctgtgaaaacgaccagtctgggaagccgggtggctgctctgacatctgcctgctggccaacagccacaaggcgcggacctgccgctgccgttccggcttcagcctgggcagtgacgggaagtcatgcaagaagccggagcatgagctgttcctcgtgtatggcaagggccggccaggcatcatccggggcatggatatgggggccaaggtcccggatgagcacatgatccccattgaaaacctcatgaacccccgagccctggacttccacgctgagaccggcttcatctactttgccgacaccaccagctacctcattggccgccagaagattgatggcactgagcgggagaccatcctgaaggacggcatccacaatgtggagggtgtggccgtggactggatgggagacaatctgtactggacggacgatgggcccaaaaagacaatcagcgtggccaggctggagaaagctgctcagacccgcaagactttaatcgagggcaaaatgacacaccccagggctattgtggtggatccactcaatgggtggatgtactggacagactgggaggaggaccccaaggacagtcggcgtgggcggctggagagggcgtggatggatggctcacaccgagacatctttgtcacctccaagacagtgctttggcccaatgggctaagcctggacatcccggctgggcgcctctactgggtggatgccttctacgaccgcatcgagacgatactgctcaatggcacagaccggaagattgtgtatgaaggtcctgagctgaaccacgcctttggcctgtgtcaccatggcaactacctcttctggactgagtatcggagtggcagtgtctaccgcttggaacggggtgtaggaggcgcaccccccactgtgacccttctgcgcagtgagcggccccccatctttgagatccgaatgtatgatgcccagcagcagcaagttggcaccaacaaatgccgggtgaacaatggcggctgcagcagcctgtgcttggccacccctgggagccgccagtgcgcctgtgctgaggaccaggtgttggacgcagacggcgtcacttgcttggcgaacccatcctacgtgcctccaccccagtgccagccaggcgagtttgcctgtgccaacagccgctgcatccaggagcgctggaagtgtgacggagacaacgattgcctggacaacagtgatgaggccccagccctctgccatcagcacacctgcccctcggaccgattcaagtgcgagaacaaccggtgcatccccaaccgctggctctgcgacggggacaatgactgtgggaacagtgaagatgagtccaatgccacttgttcagcccgcacctgcccccccaaccagttctcctgtgccagtggccgctgcatccccatctcctggacgtgtgatctggatgacgactgtggggaccgctctgatgagtctgcttcgtgtgcctatcccacctgcttccccctgactcagtttacctgcaacaatggcagatgtatcaacatcaactggagatgcgacaatgacaatgactgtggggacaacagtgacgaagccggctgcagccactcctgttctagcacccagttcaagtgcaacagcgggcgttgcatccccgagcactggacctgcgatggggacaatgactgcggagactacagtgatgagacacacgccaactgcaccaaccaggccacgaggccccctggtggctgccacactgatgagttccagtgccggctggatggactatgcatccccctgcggtggcgctgcgatggggacactgactgcatggactccagcgatgagaagagctgtgagggagtgacccacgtctgcgatcccagtgtcaagtttggctgcaaggactcagctcggtgcatcagcaaagcgtgggtgtgtgatggcgacaatgactgtgaggataactcggacgaggagaactgcgagtccctggcctgcaggccaccctcgcacccttgtgccaacaacacctcagtctgcctgccccctgacaagctgtgtgatggcaacgacgactgtggcgacggctcagatgagggcgagctctgcgaccagtgctctctgaataacggtggctgcagccacaactgctcagtggcacctggcgaaggcattgtgtgttcctgccctctgggcatggagctggggcccgacaaccacacctgccagatccagagctactgtgccaagcatctcaaatgcagccaaaagtgcgaccagaacaagttcagcgtgaagtgctcctgctacgagggctgggtcctggaacctgacggcgagagctgccgcagcctggaccccttcaagccgttcatcattttctccaaccgccatgaaatccggcgcatcgatcttcacaaaggagactacagcgtcctggtgcccggcctgcgcaacaccatcgccctggacttccacctcagccagagcgccctctactggaccgacgtggtggaggacaagatctaccgcgggaagctgctggacaacggagccctgactagtttcgaggtggtgattcagtatggcctggccacacccgagggcctggctgtagactggattgcaggcaacatctactgggtggagagtaacctggatcagatcgaggtggccaagctggatgggaccctccggaccaccctgctggccggtgacattgagcacccaagggcaatcgcactggatccccgggatgggatcctgttttggacagactgggatgccagcctgccccgcattgaggcagcctccatgagtggggctgggcgccgcaccgtgcaccgggagaccggctctgggggctggcccaacgggctcaccgtggactacctggagaagcgcatcctttggattgacgccaggtcagatgccatttactcagcccgttacgacggctctggccacatggaggtgcttcggggacacgagttcctgtcgcacccgtttgcagtgacgctgtacgggggggaggtctactggactgactggcgaacaaacacactggctaaggccaacaagtggaccggccacaatgtcaccgtggtacagaggaccaacacccagccctttgacctgcaggtgtaccacccctcccgccagcccatggctcccaatccctgtgaggccaatgggggccagggcccctgctcccacctgtgtctcatcaactacaaccggaccgtgtcctgcgcctgcccccacctcatgaagctccacaaggacaacaccacctgctatgagtttaagaagttcctgctgtacgcacgtcagatggagatccgaggtgtggacctggatgctccctactacaactacatcatctccttcacggtgcccgacatcgacaacgtcacagtgctagactacgatgcccgcgagcagcgtgtgtactggtctgacgtgcggacacaggccatcaagcgggccttcatcaacggcacaggcgtggagacagtcgtctctgcagacttgccaaatgcccacgggctggctgtggactgggtctcccgaaacctgttctggacaagctatgacaccaataagaagcagatcaatgtggcccggctggatggctccttcaagaacgcagtggtgcagggcctggagcagccccatggccttgtcgtccaccctctgcgtgggaagctctactggaccgatggtgacaacatcagcatggccaacatggatggcagcaatcgcaccctgctcttcagtggccagaagggccccgtgggcctggctattgacttccctgaaagcaaactctactggatcagctccgggaaccataccatcaaccgctgcaacctggatgggagtgggctggaggtcatcgatgccatgcggagccagctgggcaaggccaccgccctggccatcatgggggacaagctgtggtgggctgatcaggtgtcggaaaagatgggcacatgcagcaaggctgacggctcgggctccgtggtccttcggaacagcaccaccctggtgatgcacatgaaggtctatgacgagagcatccagctggaccataagggcaccaacccctgcagtgtcaacaacggtgactgctcccagctctgcctgcccacgtcagagacgacccgctcctgcatgtgcacagccggctatagcctccggagtggccagcaggcctgcgagggcgtaggttcctttctcctgtactctgtgcatgagggaatcaggggaattcccctggatcccaatgacaagtcagatgccctggtcccagtgtccgggacctcgctggctgtcggcatcgacttccacgctgaaaatgacaccatctactgggtggacatgggcctgagcacgatcagccgggccaagcgggaccagacgtggcgtgaagacgtggtgaccaatggcattggccgtgtggagggcattgcagtggactggatcgcaggcaacatctactggacagaccagggctttgatgtcatcgaggtcgcccggctcaatggctccttccgctacgtggtgatctcccagggtctagacaagccccgggccatcaccgtccacccggagaaagggtacttgttctggactgagtggggtcagtatccgcgtattgagcggtctcggctagatggcacggagcgtgtggtgctggtcaacgtcagcatcagctggcccaacggcatctcagtggactaccaggatgggaagctgtactggtgcgatgcacggacagacaagattgaacggatcgacctggagacaggtgagaaccgcgaggtggttctgtccagcaacaacatggacatgttttcagtgtctgtgtttgaggatttcatctactggagtgacaggactcatgccaacggctctatcaagcgcgggagcaaagacaatgccacagactccgtgcccctgcgaaccggcatcggcgtccagcttaaagacatcaaagtcttcaaccgggaccggcagaaaggcaccaacgtgtgcgcggtggccaatggcgggtgccagcagctgtgcctgtaccggggccgtgggcagcgggcctgcgcctgtgcccacgggatgctggctgaagacggagcatcgtgccgcgagtatgccggctacctgctctactcagagcgcaccattctcaagagtatccacctgtcggatgagcgcaacctcaatgcgcccgtgcagcccttcgaggaccctgagcacatgaagaacgtcatcgccctggcctttgactaccgggcaggcacctctccgggcacccccaatcgcatcttcttcagcgacatccactttgggaacatccaacagatcaacgacgatggctccaggaggatcaccattgtggaaaacgtgggctccgtggaaggcctggcctatcaccgtggctgggacactctctattggacaagctacacgacatccaccatcacgcgccacacagtggaccagacccgcccaggggccttcgagcgtgagaccgtcatcactatgtctggagatgaccacccacgggccttcgttttggacgagtgccagaacctcatgttctggaccaactggaatgagcagcatcccagcatcatgcgggcggcgctctcgggagccaatgtcctgacccttatcgagaaggacatccgtacccccaatggcctggccatcgaccaccgtgccgagaagctctacttctctgacgccaccctggacaagatcgagcggtgcgagtatgacggctcccaccgctatgtgatcctaaagtcagagcctgtccaccccttcgggctggccgtgtatggggagcacattttctggactgactgggtgcggcgggcagtgcagcgggccaacaagcacgtgggcagcaacatgaagctgctgcgcgtggacatcccccagcagcccatgggcatcatcgccgtggccaacgacaccaacagctgtgaactctctccatgccgaatcaacaacggtggctgccaggacctgtgtctgctcactcaccagggccatgtcaactgctcatgccgagggggccgaatcctccaggatgacctcacctgccgagcggtgaattcctcttgccgagcacaagatgagtttgagtgtgccaatggcgagtgcatcaacttcagcctgacctgcgacggcgtcccccactgcaaggacaagtccgatgagaagccatcctactgcaactcccgccgctgcaagaagactttccggcagtgcagcaatgggcgctgtgtgtccaacatgctgtggtgcaacggggccgacgactgtggggatggctctgacgagatcccttgcaacaagacagcctgtggtgtgggcgagttccgctgccgggacgggacctgcatcgggaactccagccgctgcaaccagtttgtggattgtgaggacgcctcagatgagatgaactgcagtgccaccgactgcagcagctacttccgcctgggcgtgaagggcgtgctcttccagccctgcgagcggacctcactctgctacgcacccagctgggtgtgtgatggcgccaatgactgtggggactacagtgatgagcgcgactgcccaggtgtgaaacgccccagatgccctctgaattacttcgcctgccctagtgggcgctgcatccccatgagctggacgtgtgacaaagaggatgactgtgaacatggcgaggacgagacccactgcaacaagttctgctcagaggcccagtttgagtgccagaaccatcgctgcatctccaagcagtggctgtgtgacggcagcgatgactgtggggatggctcagacgaggctgctcactgtgaaggcaagacgtgcggcccctcctccttctcctgccctggcacccacgtgtgcgtccccgagcgctggctctgtgacggtgacaaagactgtgctgatggtgcagacgagagcatcgcagctggttgcttgtacaacagcacttgtgacgaccgtgagttcatgtgccagaaccgccagtgcatccccaagcacttcgtgtgtgaccacgaccgtgactgtgcagatggctctgatgagtcccccgagtgtgagtacccgacctgcggccccagtgagttccgctgtgccaatgggcgctgtctgagctcccgccagtgggagtgtgatggcgagaatgactgccacgaccagagtgacgaggctcccaagaacccacactgcaccagcccagagcacaagtgcaatgcctcgtcacagttcctgtgcagcagtgggcgctgtgtggctgaggcactgctctgcaacggccaggatgactgtggcgacagctcggacgagcgtggctgccacatcaatgagtgtctcagccgcaagctcagtggctgcagccaggactgtgaggacctcaagatcggcttcaagtgccgctgtcgccctggcttccggctgaaggatgacggccggacgtgtgctgatgtggacgagtgcagcaccaccttcccctgcagccagcgctgcatcaacacccatggcagctataagtgtctgtgtgtggagggctatgcaccccgcggcggcgacccccacagctgcaaggctgtgactgacgaggaaccgtttctgatcttcgccaaccggtactacctgcgcaagctcaacctggacgggtccaactacacgttacttaagcagggcctgaacaacgccgttgccttggattttgactaccgagagcagatgatctactggacagatgtgaccacccagggcagcatgatccgaaggatgcaccttaacgggagcaatgtgcaggtcctacaccgtacaggcctcagcaaccccgatgggctggctgtggactgggtgggtggcaacctgtactggtgcgacaaaggccgggacaccatcgaggtgtccaagctcaatggggcctatcggacggtgctggtcagctctggcctccgtgagcccagggctctggtggtggatgtgcagaatgggtacctgtactggacagactggggtgaccattcactgatcggccgcatcggcatggatgggtccagccgcagcgtcatcgtggacaccaagatcacatggcccaatggcctgacgctggactatgtcactgagcgcatctactgggccgacgcccgcgaggactacattgaatttgccagcctggatggctccaatcgccacgttgtgctgagccaggacatcccgcacatctttgcactgaccctgtttgaggactacgtctactggaccgactgggaaacaaagtccattaaccgagcccacaagaccacgggcaccaacaaaacgctcctcatcagcacgctgcaccggcccatggacctgcatgtcttccatgccctgcgccagccagacgtgcccaatcacccctgcaaggtcaacaatggtggctgcagcaacctgtgcctgctgtcccccgggggagggcacaaatgtgcctgccccaccaacttctacctgggcagcgatgggcgcacctgtgtgtccaactgcacggctagccagtttgtatgcaagaacgacaagtgcatccccttctggtggaagtgtgacaccgaggacgactgcggggaccactcagacgagcccccggactgccctgagttcaagtgccggcccggacagttccagtgctccacaggtatctgcacaaaccctgccttcatctgcgatggcgacaatgactgccaggacaacagtgacgaggccaactgtgacatccacgtctgcttgcccagtcagttcaaatgcaccaacaccaaccgctgtattcccggcatcttccgctgcaatgggcaggacaactgcggagatggggaggatgagagggactgccccgaggtgacctgcgcccccaaccagttccagtgctccattaccaaacggtgcatcccccgggtctgggtctgcgaccgggacaatgactgtgtggatggcagtgatgagcccgccaactgcacccagatgacctgtggtgtggacgagttccgctgcaaggattcgggccgctgcatcccagcgcgttggaagtgtgacggagaggatgactgtggggatggctcggatgagcccaaggaagagtgtgatgaacgcacctgtgagccataccagttccgctgcaagaacaaccgctgcgtgcccggccgctggcagtgcgactacgacaacgattgcggtgacaactccgatgaagagagctgcacccctcggccctgctccgagagtgagttctcctgtgccaacggccgctgcatcgcggggcgctggaaatgcgatggagaccacgactgcgcggacggctcggacgagaaagactgcaccccccgctgtgacatggaccagttccagtgcaagagcggccactgcatccccctgcgctggcgctgtgacgcagacgccgactgcatggacggcagcgacgaggaggcctgcggcactggcgtgcggacctgccccctggacgagttccagtgcaacaacaccttgtgcaagccgctggcctggaagtgcgatggcgaggatgactgtggggacaactcagatgagaaccccgaggagtgtgcccggttcgtgtgccctcccaaccggcccttccgttgcaagaatgaccgcgtctgtctgtggatcgggcgccaatgcgatggcacggacaactgtggggatgggactgatgaagaggactgtgagccccccacagcccacaccacccactgcaaagacaagaaggagtttctgtgccggaaccagcgctgcctctcctcctccctgcgctgcaacatgttcgatgactgcggggacggctctgacgaggaggactgcagcatcgaccccaagctgaccagctgcgccaccaatgccagcatctgtggggacgaggcacgctgcgtgcgcaccgagaaagcggcctactgtgcctgccgctcgggcttccacaccgtgcccggccagcccggatgccaagacatcaacgagtgcctgcgcttcggcacctgctcccagctctgcaacaacaccaagggcggccacctctgcagctgcgctcggaacttcatgaagacgcacaacacctgcaaggccgaaggctctgagtaccaggtcctgtacatcgctgatgacaatgagatccgcagcctgttccccggccacccccattcggcttacgagcaggcattccagggtgacgagagtgtccgcattgatgctatggatgtccatgtcaaggctggccgtgtctattggaccaactggcacacgggcaccatctcctaccgcagcctgccacctgctgcgcctcctaccacttccaaccgccaccggcgacagattgaccggggtgtcacccacctcaacatttcagggctgaagatgcccagaggcatcgccatcgactgggtggccggaaacgtgtactggaccgactcgggccgagatgtgattgaggtggcgcagatgaagggcgagaaccgcaagacgctcatctcgggcatgattgacgagccccacgccattgtggtggacccactgagggggaccatgtactggtcagactggggcaaccaccccaagattgagacggcagcgatggatgggacgcttcgggagacactggtgcaggacaacattcagtggcccacaggcctggccgtggattatcacaatgagcggctgtactgggcagacgccaagctttcagtcatcggcagcatccggctcaatggcacggaccccattgtggctgctgacagcaaacgaggcctaagtcaccccttcagcatcgacgtctttgaggattacatctatggtgtcacctacatcaataatcgtgtcttcaagatccataagtttggccacagccccttggtcaacctgacagggggcctgagccacgcctctgacgtggtcctttaccatcagcacaagcagcccgaagtgaccaacccatgtgaccgcaagaaatgcgagtggctctgcctgctgagccccagtgggcctgtctgcacctgtcccaatgggaagcggctggacaacggcacatgcgtgcctgtgccctctccaacgccccccccagatgctccccggcctggaacctgtaacctgcagtgcttcaacggtggcagctgtttcctcaatgcacggaggcagcccaagtgccgctgccaaccccgctacacgggtgacaagtgtgaactggaccagtgctgggagcactgtcgcaatgggggcacctgtgctgcctccccctctggcatgcccacgtgccggtgccccacgggcttcacgggccccaaatgcacccagcaggtgtgtgcgggctactgtgccaacaacagcacctgcactgtcaaccagggcaaccagccccagtgccgatgcctacccggcttcctgggcgaccgctgccagtaccggcagtgctctggctactgtgagaactttggcacatgccagatggctgctgatggctcccgacaatgccgctgcactgcctactttgagggatcgaggtgtgaggtgaacaagtgcagccgctgtctcgaaggggcctgtgtggtcaacaagcagagtggggatgtcacctgcaactgcacggatggccgggtggcccccagctgtctgacctgcgtcggccactgcagcaatggcggctcctgtaccatgaacagcaaaatgatgcctgagtgccagtgcccaccccacatgacagggccccggtgtgaggagcacgtcttcagccagcagcagccaggacatatagcctccatcctaatccctctgctgttgctgctgctgctggttctggtggccggagtggtattctggtataagcggcgagtccaaggggctaagggcttccagcaccaacggatgaccaacggggccatgaacgtggagattggaaaccccacctacaagatgtacgaaggcggagagcctgatgatgtgggaggcctactggacgctgactttgccctggaccctgacaagcccaccaacttcaccaaccccgtgtatgccacactctacatggggggccatggcagtcgccactccctggccagcacggacgagaagcgagaactcctgggccggggccctgaggacgagataggggaccccttggcatag

SEQ ID NO: 14 - protein, 4 aa, Artificial Sequence
  exemplary motif
NICD

SEQ ID NO: 15 - protein, 6 aa, Artificial Sequence
  exemplary motif
YENPTY

SEQ ID NO: 16 - protein, 4 aa, Artificial Sequence
  exemplary motif
NPVY

SEQ ID NO: 17 - protein, 4 aa, Artificial Sequence
  exemplary motif
YATL

SEQ ID NO: 18 - protein, 7 aa, Artificial Sequence
  exemplary motif
NPVYATL

SEQ ID NO: 19 - protein, 8 aa, Artificial Sequence
  exemplary motif
NPVYYATL

SEQ ID NO: 20 - protein, 4 aa, Artificial Sequence
  exemplary motif
NPTY

SEQ ID NO: 21 - protein, 6 aa, Artificial Sequence
  exemplary motif
IGNPTY

SEQ ID NO: 22 - protein, 6 aa, Artificial Sequence
  exemplary motif
FTNPVY

SEQ ID NO: 23 - protein, 4 aa, Artificial Sequence
  exemplary motif
YENP

SEQ ID NO: 24 - protein, 6 aa, Artificial Sequence
  exemplary motif
XENPXY

SEQ ID NO: 25 - protein, 7 aa, Artificial Sequence
  exemplary motif
NPTYATL

SEQ ID NO: 26 - protein, 100 aa, Homo sapiens
KRRVQGAKGFQHQRMTNGAMNVEIGNPTYKMYEGGEPDDVGGLLDADFALDPDKPTNFTNPVYATLYMGGHGSRHSLASTDEKRELLGRGPEDEIGDPLA

(19) United States
(12) Patent Application Publication
(10) Pub. No.: US 2004/0038338 A1
(43) Pub. Date: Feb. 26, 2004

(54) Influence of LRP cytoplasmic domain on Abeta production

(76) Inventors: Edward H. Koo, La Jolla, CA (US); Claus Pietrzik, Nierstein (DE)

Correspondence Address: FISH & RICHARDSON, PC, 12390 EL CAMINO REAL, SAN DIEGO, CA, 92130-2081, US

(21) Appl. No.: 10/210,175

(22) Filed: Jul. 31, 2002

Related U.S. Application Data
(60) Provisional application No. 60/309,249, filed on Jul. 31, 2001.

(51) Int. Cl. C07K 14/705; C07H 21/04; C12P 21/02; C12N 5/06

(52) U.S. Cl. 435/69.1; 435/320.1; 435/325; 530/350; 536/23.5

(57) ABSTRACT

A truncated dominant negative mammalian LDL receptor related protein (LRP) cytoplasmic tail mutant (LRP-CT) molecule and DNA sequences for its construction is described in this disclosure as is a method for disrupting generation of amyloid β-protein (Aβ). Methods for preventing or treating diseases wherein amyloid β-protein (Aβ) is a major constituent of amyloid plaques or amyloidosis by interfering with production of Aβ are described, as is a high throughput assay for screening compounds that inhibit Aβ production. Also described is a method for inhibiting LRP or APP:Fe65 interaction in vivo, and kit suitable for providing the required reactants for screening assays.

This application claims priority to U.S. Provisional application No. 60/309,249, filed Jul. 31, 2001.

This work was supported in part by National Institutes of Health No. AG12376. The U.S. Government may have some interest in this application.

### BACKGROUND OF THE INVENTION

1. Field of the Invention

The present invention generally concerns compositions and methods for disrupting the production of Aβ peptides.

The present invention particularly concerns a new method and procedure for preventing the formation of Aβ-containing plaques associated with Alzheimer's Disease. More specifically, a truncated LRP cytoplasmic tail mutant that is capable of inhibiting production of Aβ peptide is described.

2. Description of Related Art

Senile Plaques, Aβ and AD.

The pathological hallmarks of Alsheimer's Disease (AD), the most common age-related neurodegenerative disorder, are the presence of neurofibrillary tangles and extracellular deposits of amyloid β-protein (Aβ) in senile plaques in cerebral cortex [Katzman R. New Engl. J. Med. 314:964-973, 1986, Tomlinson B E. Neuropath. Appl. Neurobiol. 15: 491-512, 1989]. Although these brain lesions are seen in aged non-demented individuals, the deposition of amyloid is increasingly viewed as the earliest histopathologic event in the pathogenesis of AD. This view is at the heart of the decade old “amyloid cascade hypothesis” which states that neuronal dysfunction and death, synapse loss, neurofibrillary degeneration, microglial activation and the full manifestation of Alzheimer pathology is initiated by abnormal Aβ processing [Selkoe D J. Physiol Rev. 81:741-766, 2001]. Increasing evidence in the past several years argues that Aβ42, the 42 amino acid Aβ isoform, derived from the amyloid precursor protein (APP) is the peptide that is initially deposited in brain and hence the pathogenic Aβ species [De Strooper B and Annaert W. J. Cell Sci. 113:1857-1870, 2000]. The shorter Aβ40 is the more abundant species and is constitutively produced both in vitro and in vivo. However, Aβ42 aggregates more readily than Aβ40 in vitro, is consistently more abundant than Aβ40 in brain, and oligomers and aggregated Aβ is toxic to a variety of cells in culture [Yankner B A. Neuron. 16:921-932, 1996, Teplow D B. Amyloid. 5:121-142, 2002]. All genetic mutations that are causative of familial AD are consistently associated with perturbations in Aβ peptides levels, most commonly with a selective increase in the levels of Aβ42 peptides [Selkoe D J. Physiol Rev. 81:741-766, 2001]. This latter finding has been seen in all PS1 and PS2 mutations analyzed to date [Murayama O, et al. Neurosci. Lett. 265:61-63, 1999].

Aβ and APP Biology.

The APP gene is located on chromosome 21 and triplication of this gene appears to be necessary for Down's syndrome affected individuals to develop AD pathology [Prasher V P, et al. Ann. Neurol. 43:380-383, 1998]. APP is part of a family of three proteins, including APLP1 and APLP2, neither of which contain the Aβ sequence [De Strooper B and Annaert W. J. Cell Sci. 113:1857-1870, 2000]. All are type I membrane proteins with a large extracellular domain and a short cytoplasmic region. The mature APP molecule contains a signal peptide, N- and O-linked carbohydrates, tyrosine sulfation and phosphorylation sites, the latter acquired as it transits the endoplasmic reticulum (ER) and Golgi apparatus. Alternative splicing generates a number of APP isoforms, the major species being the Kunitz protease inhibitor domain (KPI), APP751 and APP770, and the neuron-specific form without the KPI domain (APP695). In neurons, APP is transported in axons via the fast anterograde and retrograde components [Koo E H, et al. Proc. Natl. Acad. Sci. USA. 87:1561-1565, 1990, Sisodia S S, et al. J. Neurosci. 13:3136-3142, 1993] where it interacts with kinesin light chain and may be a cargo receptor for kinesin-1 [Kamal A, et al. Neuron. 28:449-459, 2000].

Both Aβ40 and Aβ42 peptides are derived by proteolysis from APP. APP undergoes constitutive cleavage by α-secretases (TACE and ADAM family of proteases) releasing the large soluble N-terminal derivative, APPs□ [Buxbaum J D, et al. J. Biol. Chem. 273:27765-27767, 1999, Lammich S, et al. Proc. Natl. Acad. Sci U.S.A. 96:3922-3927, 1999]. This cleavage event takes place both intracellularly and on the cell surface. Significantly, the cleavage site occurs within the Aβ domain and thus generation of APPsα precludes the formation of full length Aβ peptides [Sisodia S S, et al. Science. 248:492-495, 1990]. APP molecules can alternatively be cleaved by β-secretase or BACE, identified almost simultaneously by several laboratories as a novel membrane bound aspartyl protease (Asp2, memepsin), generating the N-terminus of A [Sinha S, et al. Nature. 402:537-540, 1999, Vassar R, et al. Science. 286:735-741, 1999, Yankner B A. Neuron. 16:921-932, 1996]. BACE is localized primarily to the trans-Golgi network (TGN) and in endosomes, where the cleavage event is believed to take place [Dingwall C. J. Clin. Invest. 108:1243-1246, 2001, Huse J T, et al. J. Biol. Chem. 275:33729-33737, 2000]. Whether BACE activity accounts for the low levels of Aβ formed in the early compartment (ER and intermediate compartment) that may not be secreted is not resolved. A final cleavage at the C-terminal end by an as yet unidentified proteolytic activities within the transmembrane region generates and releases Aβ from APP [De Strooper B and Annaert W. Nature Cell Biology. 3:E221-E225, 2001]. This enigmatic intramembrane cleavage event has been designated as γ-secretase and requires the presence of presenilin-1 (PS1) and to a lesser extent presenilin-2 (PS2) as well as nicastrin [Yu G, et al. Nature. 407:48-54, 2000]. Presenilins are also required for the intramembranous cleavage of notch receptors, which releases the intracellular signalling domain, NICD [De Strooper B, et al. Nature. 398:518-522, 1999]. Several lines of indirect evidence have suggested that PS1 may itself be the elusive γ-secretase but direct proof is lacking and the issue remains unresolved [Esler W P, et al. Nat. Cell Biol. 2:428-434,2000, Li Y M, et al. Proc. Natl. Acad. Sci U.S.A. 97:6138-6143, 2000, Li Y M, et al. Nature. 405:689-694, 2000, Wolfe M S, et al. Nature. 398:513-517, 1999]. Significantly, the subcellular localization of presenilins and the compartments where Aβ is formed do not overlap completely, a mismatch that has been termed a spatial paradox” and remains to be resolved [Cupers P, et al. J. Cell Biol. 154:731-740, 2001]. Importantly, whether the different Aβ C-termini result from the activities of one or more γ-secretases is unknown. In addition, how APP and presenilin (PS) mutations alter γ-secretase activity in ways to selectively increase the Aβ42 isoform is unclear.

APP is Internalized.

In addition to the constitutive secretion of APPs following □-secretase cleavage, full-length APP molecules at the cell surface can be rapidly endocytosed without secretion or cleavage [Haass C, et al. Nature. 357:500-503, 1992]. This internalization is mediated through the -YENPTY- motif in the cytoplasmic tail of APP, a consensus sequence for receptor mediated endocytosis [Koo E H and Squazzo S L. J. Biol. Chem. 269:17386-17389, 1994, Perez R G, et a. J. Biol. Chem. 274:18851-18856, 1999]. Consistent with this finding, APP is localized to clathrin coated vesicles and early endosomes [Marquez-Sterling N R, et al. J. Neurosci. 17:140-151, 1997, Yamazaki T, et at. J. Cell Science. 109:999-1008, 1996]. After endocytosis, some of the APP molecules are targeted to late endosomes and lysosomes, while others recycle to the cell surface. Recent studies have also localized APP to cholesterol-rich microdomains on the plasma membrane (lipid rafts) where it may co-localize with caveolin-1 [Ikezu T, et al. J. Biol. Chem. 273:10485-10495, 1998, Lee S-J, et al. Nature Medicine. 4:730-734, 1999, Parkin E T, et al. Biochem. J. 344 Pt 1:23-30, 1999, Riddell D R, et al. Current Biology. 11: 1288-1293, 2001]. Thus, APP is trafficked through both the canonical clathrin-mediated endocytic pathway as well as in the caveolar pathway. Inventors have previously proposed that the internalization pathway is essential to Aβ production [Koo E H and Squazzo S L. J. Biol. Chem. 269:17386-17389, 1994]. Inventors' initial speculation was based on the simple observation that APP was internalized without secretion in this endocytic pathway and that potentially amyloidogenic C-terminal fragments were recovered in purified lysosomes. However, most investigators now agree that lysosomes are not a major and perhaps even minor site for Aβ generation. By multiple approaches, Inventors have provided strong evidence that endocytic processing is indeed one route of Aβ formation, although as mentioned above, some Aβ species are clearly derived from the early secretory pathway, i.e. without targeting to the cell surface. Recent studies have also suggested that Aβ can be generated from cholesterol-rich microdomains (lipid rafts or detergent insoluble glycolipid-enriched fraction [DIG]) [Lee S-J, et al. Nature Medicine. 4:730-734, 1999, Morishima-Kawashima M and Ihara Y. Biochemistry. 37:15247-15253, 11-3-1998]. The latter conclusion derives from the findings that APP, presenilin, BACE, and Aβ can be found in these detergent insoluble compartments [Riddell D R, et al. Current Biology. 11:1288-1293, 2001], an interpretation that is in agreement with the association of cholesterol with AD risk [Wolozin B. Proc. Natl. Acad. Sci U.S.A. 98:5371-5373, 2001] and the modulation of Aβ production by changing cholesterol levels [Fassbender K, et al. Proc. Natl. Acad. Sci U.S.A. 98:5856-5861, 2001, Kojro E, et al. Proc. Natl. Acad. Sci U.S.A. 98:5815-5820, 2001, Simons M, et al. Proc. Natl. Acad. Sci. USA. 95:6460-6464, 1999, Simons M, et al. 57:1089-1093, 2001]. In one proposed model, BACE is present in rafts together with a small fraction of APP but the localization as well as secretase activities can be shifted depending on the cholesterol state of the cell. Importantly, the relative contribution of each of these pathways to overall generation and release of Aβ is not clear. Inventors favor a primary role for the endocytic pathway in the production of Aβ from wild type APP expressed in neurons and in non neural cells, an interpretation supported by studies from other laboratories [Cescato R, et al. J. Neurochem. 74:1131-1139, 2000, Cupers P, et al. J. Cell Biol. 154:731-740, 2001, LeBlanc A C and Gambetti P. Biochem. Biophys. Res. Commun. 204:1371-1380, 1994]. On the other hand, APP containing the Swedish mutation skews the generation of Aβ generation into the secretory pathway because the mutation renders APP a substantially better substrate for BACE [Citron M, et al. Nature. 360:672-674, 1992, Perez R G, et al. J. Biol. Chem. 271:9100-9107, 1996, Thinakaran G, et al. J. Biol. Chem. 271:9390-9397, 1996]. In this setting, the need for endocytic processing to generate α- and γ-secretase cleavages is negated. The issue of lipid rafts remains somewhat controversial and it is unclear where they fit into the aforementioned scheme.

LDL Receptor-Related Protein (LRP) Influences Aβ Production.

As briefly described above, there is substantial evidence to support the generation of Aβ in early compartments, in the endocytic pathway, and recently, in lipid rafts. However, how the production of Aβ may be regulated, beyond identification of BACE and γ-secretase activities, and which protein partners are necessary for Aβ generation, is unknown. Accumulating evidence favors a role of the low density lipoprotein receptor-related protein (LRP) in AD pathogenesis [Hyman B T, et al. Arch. Neurol. 57:646-650, 2000]. LRP is a member of the low density lipoprotein (LDL) receptor family of endocytic receptors. Although structurally similar to other members of the LDL receptor (LDLR) gene family, LRP is considerably larger [Krieger M and Herz J. Ann. Rev. Biochem. 63:601-637, 1994], synthesized as a 600 kDa transmembrane glycoprotein that is cleaved in the TGN by furin to generate a 515 kDa α- and an 85 kDa β-subunit [Herz J, et al. EMBO J. 9:1769-1776, 1990]. The subunits remain associated in a noncovalent fashion as they are routed to the cell surface [Willnow T E, et al. EMBO J. 15:2632-2639, 1996]. From there, LRP recycles between endosomal locations and the cell surface. More than 19 ligands, including the KPI-containing APP isoform, have been reported to bind to the large 515 kDa N-terminal fragment of LRP, which can be divided into four ligand binding domains [Bu G and Rennke S. J. Biol. Chem. 271:22218-22224, 1996, Mikhailenko I, et a. J. Biol. Chem. 276:39484-39491, 2001, Moestrup K and Gliemann J. J. Biol. Chem. 266:14011-14017, 1991, Strickland D K, et al. J. Biol. Chem. 266:13364-13369, 1991, Willnow T E, et al. J. Biol. Chem. 269:15827-15832, 1994], but not to the 85 kDa C-terminal β-subunit of LRP. While the LDLR appears to function exclusively in lipoprotein metabolism, increasing evidence suggest that LRP and the other members of the gene family have diverse biological roles, including signal transduction and neurotransmission [Herz J and Strickland D K. J. Clin. Invest. 108:779-784, 2001]. In the cytoplasmic region of 100 amino acids are two NPXY motifs for endocytosis and interaction with adaptor molecules [Herz J, et al. EMBO J. 7:4119-4127, 1988]. LRP complexes with various cytoplasmic adaptor proteins such as Fe65, Disabled-1 (mDab1), and signaling intermediates such as JIP-1 (JNK interacting protein) [Gotthardt M, et al. J. Biol. Chem. 275:25616-25624, 2000, Howell B W, et al. Mol. Cell Biol. 19:5179-5188, 1999]. Remarkably, LRP and three of its ligands: APP [Kounnas M Z, et al. Cell. 82:331-340, 1995], apolipoprotein E, and α-2-macroglobulin (α2M), are all genetically associated with late-onset AD [Hyman B T, et al. Arch. Neurol. 57:646-650, 2000]. LRP has been shown to mediate the clearance of Aβ-α2M complexes in vitro and in vivo [Narita M, et al. J. Neurochem. 69:1904-1911, 1997, Shibata M, et al. J. Clin. Invest. 106:1489-1499, 2000]. Inventors recently proposed that the levels of LRP in brain may contribute to AD pathogenesis by modulating the clearance, and hence levels, of Aβ in vivo [Kang D E, et al. J. Clin. Invest. 106:1159-1166, 2000]. In addition to mediating Aβ clearance, LRP was recently shown to influence Aβ production. Aβ generation and secretion was dramatically reduced in cells lacking LRP and overexpressing the KPI-containing APP751 isoform [Ulery P G, et al. J. Biol. Chem. 275:7410-7415, 2000]. Furthermore, treatment with exogenous 39 kDa receptor associated protein (RAP), an LRP chaperone molecule that blocks all binding of all known LRP ligands, also reduced Aβ release. At the same time, secretion of APPsα was increased significantly, indicating a shift towards the nonamyloidogenic pathway. Therefore, Ulery and colleagues proposed that the association of APP via the KPI domain at the cell surface with LRP modulates APP internalization and subsequent Aβ production [Ulery P G, et al. J. Biol. Chem. 275:7410-7415, 2000].

The breakdown of APP leads to the production of Aβ peptide, which is believed to at least participate in causing Alzheimer's Disease. The correct mechanism by which the APP is internalized and broken down has not heretofore been elucidated. In U.S. Pat. No. 6,156,311, issued to Strickland, there is described a method of attack against formation of the Amyloid placques and AD, however, the method involves incorrect assumptions and therefore is ineffective, except to a non-specific degree. The disclosure therein assumes that secreted forms of APP are generated by proteolytic cleavages within their extracellular domain close to the transmembrane region. This now is known not to be true, as disclosed herein. There is, therefore, a great need to discover the mode of interaction between APP and LRP, and to develop effective methods, assays and pharmaceutical compositions in order to wage war against the severely debilitating amyloid diseases.

### SUMMARY OF THE INVENTION

The primary object of this invention is to provide effective compositions and methods for treating amyloid diseases such as Alzheimer's.

Another object in accordance with the present invention is to provide a high throughput system to screen for appropriate molecules that are capable of inhibiting the formation of APP:Fe65:LRP complexes that result in triggering a cascade of events to cause plaque formation.

A further, most preferred object is to provide a rational approach to the treatment of diseases involving Aβ protein.

In accordance with these objects, this invention contemplates a truncated dominant negative mammalian LDL receptor related protein (LRP) cytoplasmic tail mutant (LRP-CT) molecule, having a signal peptide and about 100 to 400 amino acids at the cytoplasmic tail end of LRP. This fragment encompasses the entire transmembrane and cytoplasmic domains, and has a deletion of 22 amino acids, from residue number 4486 to residue number 4507, from an LRP molecule having 4544 amino acid residues. The deletion encompasses the domain that is critical in processing amyloid precursor protein (APP).

The contemplated method further involves a DNA sequence for constructs for expressing truncated LDL receptor related protein (LRP) cytoplasmic tail mutant (LRP-CT) molecule selected from the group consisting of SEQ ID NO 1, SEQ ID NO 3, SEQ ID NO 4, SEQ ID NO 5, SEQ ID NO 6, SEQ ID NO 7, SEQ ID NO 8, SEQ ID NO 9, SEQ ID NO 10, SEQ ID NO 11, and SEQ ID NO 12.

An equally preferred embodiment in accordance with this invention is a method for disrupting generation of amyloid β-protein (Aβ) by constructing a truncated LDL receptor related protein cytoplasmic tail mutant (LRP-CT). The mutant is introduced and overexpressed in a normal mammalian cell. Subsequently, the mutant is allowed to react with an amyloid precursor protein (APP) that is required for producing Aβ and that is produced by the cell. The reacting of the LRP-CT mutant protein with the APP reactive site competes with LRP resulting in diminishing formation of an APP:LRP complex, thereby interfering with enzymatic processing of APP that is required in Aβ production.

The interaction that forms the complex requires a third, adaptor, molecule. One such adaptor is a molecule known as Fe65. The inhibition can be effectuated by a functional analogue of the truncated LRP-CT molecule. In that case, the analogue of the truncated LRD-CT molecule is selected from the group comprising reagents, drugs, compounds peptides, peptidomimetics and antibodies to the reactive site of either APP or LRP cytoplasmic tail region.

A most preferred embodiment in accordance with this invention is a method for preventing or treating diseases wherein amyloid β-protein (Aβ) is a major constituent of amyloid plaques or amyloidosis by interfering with production of Aβ. The method preferably entails contacting the affected cells or tissues of a recipient with the truncated dominant negative LDL receptor related protein cytoplasmic tail mutant (LRP-CT) described above, or an analogue thereof. The inhibitor is permitted to internalize into the target cells, allowing it to interact with APP or an adaptor molecule thereby binding to the APP or adaptor molecule. The binding competes with wild-type APP and interferes with enzymic processing of the APP into Aβ, preventing the triggering of a cascade of molecular events causing amyloid plaque formation found in these diseases. The recipient's Aβ profile can be monitored throughout the treatment.

Another most preferred embodiment is a high throughput assay for screening compounds that inhibit Aβ production, combining a substrate molecule, a test molecule and a reporter molecule in an appropriate reaction chamber, and permitting the reaction to proceed for an appropriate time. The loss of binding capability by the reporter molecules can then be easily detected, and represents interference and loss of the LRP (or APP):Fe65 interaction due to competition by the candidate test molecule.

The loss of interaction can be between APP and Fe65 or between LRP and Fe65. The reporter molecule can be labeled to facilitate detection of binding. The label can be one of a number of fluorescent dyes, enzyme reaction products, radioisotopes or other suitable markers known to those experienced in the field.

It is important to note the method can be practiced by impairing formation of an LRP-Fe65-APP trimeric complex by blocking active receptor sites of any of the three components capable of forming the complex.

The impairing can be by a truncated cytoplasmic tail of LRP. Especially preferred is impairment by the truncated cytoplasmic tail of LRP having sequences SEQ ID NOS 1, 2-8 described herein, and their analogs or functional equivalents. The impairing can also be accomplished by a small, functionally analogous molecules such as peptides, peptidomimetics, drugs or antibodies to the active site.

Most preferred is a method for inhibiting LRP or APP:Fe65 interaction in vivo by administering to a recipient an effective amount of inhibitor compound to prevent the formation of LRP-Fe65-APP trimeric complexes facilitating catabolism of APP to produce Aβ.

The administering can be orally, as by taking an effective drug in liquid or tablet form. The administering can also be by injection, intravenously, intraperitoneally or intracranially. Other methods include administering is by infusion intravenously, by gene therapy or by implanting into a recipient a time-release mechanism or substance. The molecules can be designed to facilitate treatment of the brain so that they can permeate the blood/brain barrier. These techniques are known to those experienced in the field.

Also highly preferred is a kit suitable for providing the required reactants for screening assays. The kit may have a vessel of appropriate size containing APP, a vessel containing an appropriate amount of Fe65 adaptor peptide, a signal substance and instructions, including an appropriate standardized profile chart.

Still further embodiments and advantages of the invention will become apparent to those skilled in the art upon reading the entire disclosure contained herein.

### BRIEF DESCRIPTION OF THE DRAWINGS

FIG. 1 is a diagramatic representation of the model for the assembly and internalization of APP and LRP according to this invention.

FIG. 2 shows APPs and APP-CTF levels in LRP−/− cells.

Mouse embryonic fibroblasts lacking the LRP gene (LRP−/−) and corresponding LRP expressing control fibroblasts (LRP±) were stably transfected with APP751. APPs was immunoprecipitated using the monoclonal antibodies 1G7-5A3 and the samples immunoblotted with an APP polyclonal antibody (863) as described in the Methods section. a) LRP deficient cells secrete approximately 2.5 fold more APPs then LRP expressing cells. Results are expressed as average of two experiments in triplicate ±SEM. b) APP expression in LRP± and LRP−/− fibroblasts was determined by immunoblotting with a polyclonal antibody (CT15) raised against the C-terminus of APP. Single clones of similar APP expression were selected (top panel). In LRP−/− cells, there is a dramatic reduction in the level of APP-CTF (bottom panel).

FIG. 3 shows the turnover of full-length APP and APP-CTF in LRP−/− cells.

LRP± and LRP−/− fibroblasts were pulse-labeled with [35S] methionine/cysteine for 15 min and chased for 0, 1, 2, and 4 hours (a). At time 0, APP consists predominantly of immature N-glycosylated species. Both the N-glycosylated and mature N- and O-glycosylated species are abundant at 1 hour for both cell types. After 2 hours chase period, APP level is dramatically reduced in LRP± cells and hardly detectable at 4 hours. In contrast, APP can still be detected in LRP−/− cells even after a four hour chase period. b) Half-life was determined by quantitating the results from (a) as shown. c) APP-CTF turnover was determined metabolically labeling LRP± and LRP−/− fibroblasts with [35S] methionine/cysteine for 1 hour and chased for 3, 6, and 18 hours (c). Similar amounts of APP-CTF's are present after the labeling and after 3 hours chase period in LRP± and LRP−/− cells. Even after an 18 hours chase period APP-CTF's can still be detected in LRP± fibroblasts. However, APP-CTF's in LRP−/− fibroblasts were almost completely degraded. d) Half-life of APP-CTF was determined by quantitating the results from (c) as shown. Experiment a) was performed in triplicate and c) in duplicate and representative experiments are shown.

FIG. 4 depicts APP processing in CHO cell lines.

a) Endogenous full-length APP (upper panel) and APP-CTF (lower panel) were immunoblotted with APP C-terminal antibody (CT15) from CHO cell line 13-5-1 deficient in LRP, CHO cell line 14-2-1 expressing a mutant LRP defective in trafficking to the plasma membrane, and control CHO-K1 cell line. Note that cells lacking LRP (13-5-1) show a significant reduction in the level of APP-CTF (bottom panel) while the cells expressing the LRP trafficking mutant (14-2-1) show higher endogenous APP-CTF levels. b) LRP is detected by immunoblotting with the polyclonal antibody 1704 raised against the C-terminus of LRP. As expected, no LRP can be seen in CHO cell line 13-5-1 (middle lanes) while the amount of β-subunit in CHO 14-2-1 cells is significantly less than in control cells ratio indicating lack of furin cleavage due to retention in the early secretory compartments. Uncleaved full length LRP (α+β) can be visualized and the levels are comparable between CHO 14-2-1 and CHO K1 control cells. c) Secretion of endogenous APPs into medium was increased in both CHO 13-5-1 and CHO 14-2-1 cells as compared to control CHO K1 cells.

FIG. 5 indicates that loss of APP-CTF in LRP−/− cells is isoform independent and can be restored by expression of LRP-CT construct.

a) LRP± and LRP−/− cells stably transfected with APP695 were analyzed for levels of APP-CTFs. Single clones of similar APP expression were selected for analysis (top panel). Note that LRP−/− fibroblasts expressing APP695 show a dramatic reduction in APP-CTF levels expression (bottom panel). b) LRP−/− fibroblasts transfected with APP751 were infected with a retrovirus expressing LRP-CT construct and immunoblotted with the LRP C-terminal antibody 1704. No signal is seen in the LRP−/− cells (middle lanes, top panel). The LRP-CT protein is a truncated β-subunit (right lanes LRP−/− LRP-CT) and migrates faster than the authentic β-subunit (left lanes, LRP±) and as expected, the full length LRP species (α+β) is absent in the LRP−/−; LRP-CT cells (right lanes, top panel). In the bottom panel, immunoblotting of APP-CTF was carried out with the APP C-terminal antibody CT15. Note that following expression of LRP-CT construct in LRP−/− cells, the normally low levels of APP-CTF in LRP−/− cells (middle lanes, bottom panel) are now restored to the level seen in LRP± cells (compare right with left lanes, bottom panel). c) Levels of APPs in medium of LRP±, LRP−/− and LRP−/−; LRP-CT cells were determined by immunoprecipitation-western blotting as before. As with APP-CTF levels, expression of LRP-CT in LRP−/− cells restores the abnormal levels of APPs in LRP−/− cells. In this case, the APPs are decreased after introduction of LRP-CT in LRP−/− cells. d) LRP-CT is transported to the cell surface. LRP±, LRP−/− and LRP−/− LRP-CT cells were surface biotinylated, precipitated with streptavidin beads and blotted for LRP. Note that endogenous full length (α+βsubunit) LRP was detected only in cell lysates but not biotinylated samples from LRP± cells, indicating that only proteins at the cell surface were labeled.

FIG. 6 shows Aβ, cell surface APP, and APP internalization in LRP−/− cells.

a) Reduction in Aβ release in LRP−/− cells is rescued by expression of LRP-CT construct. Aβ was measured by ELISA from LRP−/−, LRP−/−; LTP-CT, and LRP± cells after 72 hour collection. Aβ level is reduced five fold in LRP−/− cells transfected with APP751 as compared to control LRP± cells transfected with APP751, normalized for level of APP expression. The reduction in Aβ in LRP−/− cells is restored to control level after expression of LRP-CT construct. b) Cell surface APP was measured by incubating the radio iodinated 1G7 antibody with the indicated cells at 4° C. The radioactivity from acidic labile washes (see methods) is a measure of cell surface APP. Note that lack of LRP expression does not alter the amount of APP at the cell surface, nor does expression of LRP-CT in LRP−/− cells. c and d) Internalization of APP from the cell surface was measured with radio iodinated 1G7 antibody at 37□C. (see methods). LRP−/− cells stably transfected with APP751 (c) or APP695 (d) show 50% reduction in APP endocytosis as compared to control (LRP±) cells. c) The reduction in APP internalization in LRP−/− cells is similarly restored after expression of LRP-CT construct. All the graphs in this figure show results (average ±SEM) of representative experiments performed in triplicate. Statistical Analysis was performed using Students T-Test (*p<0.05).

FIG. 7 demonstrates that the region around the second NPXY domain in LRP regulates APP processing.

Two LRP-CT deletion constructs: LRP-CTΔ1 (lacking amino acid residues 4469-4484) or LRP-CTΔ2 (lacking amino acid residues 4486-4507), are schematically illustrated (a) and expressed in LRP−/− fibroblasts. b) Expression of the LRP-CTΔ1 and LRP-CTΔ2 in LRP−/− is comparable to each other and to endogenous level as seen by Immunoblotting with LRP antibody 1704 (upper panel). The reduction in the levels of APP-CTFs in LRP−/− cells is restored by expression of LRP-CTΔ1 but not LRP-CTΔ2 construct. c) Consistent with (b), APPs secretion was restored to control LRP± levels only in the LRP-CTΔ1 transfected cell line but not in the LRP-CTΔ2 cell line. d) LRP-CTΔNPVY (lacking amino acid residues 4504-4507), LRP-CTΔYATL (lacking amino acid residues 4507-4510) were transiently expressed in LRP deficient CHO 13-5-1 (see FIG. 7a for schematic). Similar to LRP-CTΔ2, expression of neither LRPCT ΔNPVY nor LRP-CTΔYATL was able to restore the reduction in APP-CTF levels (d, top) or the increase in APPs (d, bottom) to that seen in control K1 cells. In contrast, the expression of LRP-CT is able to restore the deficiencies in APP processing in LRP−/− CHO 13-5-1 cells to control levels.

FIG. 8 is a schematic representation of a model for the assembly and internalization of APP and LRP.

It has been demonstrated that isoforms of APP that contain the Kunitz-type protease inhibitor domain can directly interact with the extracellular domain of LRP (A). However, these APP isoforms are expressed only at low levels in neurons. As shown in FIG. 6 the region around the second NPXY domain of the cytoplasmic tail of LRP might form a complex with APP through adaptor proteins regulating APP processing. Our results show that, regardless of the presence of a Kunitz domain, LRP and APP can also interact through their cytoplasmic tails, suggesting that LRP can modulate the intracellular trafficking of APP.

FIG. 9 contains panels a-f, referring to data collected that is not presented in the Description part of the text, but is presented here in its entirety.

A: Inventors assayed for APP CTF in LRP± and LRP−/− cells stably transfected with either APP695 or APP751. The expression levels of APP in −/− and control ± cells were comparable (Panel A top). Interestingly the levels of the α-secretase cleaved membrane remaining stub of APP (C83) were consistently and significantly decreased in LRP−/− cells as compared to controls (Panel A, arrow). More interestingly, this decrease occurred in the LRP−/− cells independent of whether the APP695 or APP751 isoform was expressed. The isoform independence indicated to Inventors that the KPI domain is probably not responsible for this marked difference in CTF generation. To Inventors' knowledge, this is the first demonstration of APP695 processing is affected by LRP and may represent a consequence of LRP:APP interaction.

In view of the idea that the cytoplasmic domains of LRP and APP may interact with each other, Inventors engineered a truncated LRP construct consisting of the signal peptide and the last amino acids of LRP which encompasses the entire transmembrane and cytoplasmic domains (LRP-CT). When this construct, LRP-CT, was expressed in LRP deficient cells, Inventors' preliminary analysis showed remarkably that the levels of APP CTF were restored (Panel A right panel). This is an exciting preliminary observation because it is consistent with Inventors' hypothesis that the cytoplasmic domain of LRP modulates APP processing. To Inventors' knowledge this is the first demonstration of a change in APP processing, which is KPI-domain independent, but dependant on the LRP cytoplasmic domain.

B: When the cell lysates were normalized to both protein and APP levels, the amount of APPs in LRP−/− cells was estimated to be two to three fold higher than LRP± cell (Panel B). After stable transfection of the LRP-CT fragment APPs secretion was restored to normal levels.

C: Note that the turnover rate of APP in LRP−/− cells is dramatically altered compared to LRP expressing LRP± cells.

D: It has been shown that endocytic processing is a major pathway for the production and subsequent release of Aβ into medium. Inventors therefore assessed the level of endocytosis in LRP−/− and LRP± cells stable transfected with APP685 or APP751 constructs. The levels of APP that has been internalized compared to cell surface molecules can be rapidly determined by the acid resistant and acid labile pools, respectively. In the LRP± cells transfected with APP695 or 751, APP was rapidly internalized from the cell surface, as has been shown for other cell types (data not shown). In contrast, after 30 minutes, the internalized pool of APP in LRP−/− cells was reduced by approximately 40% as compared to LRP± cells (Panel D). Significantly, this result was seen in both APP695 and APP751 expressing cells.

E: Aβ was measured from serum free medium after 48 and 72 h ours incubation of LRP± and LRP−/− cells stably transfected with APP751. Media samples were measured by ELISA (by Dr. Maria Kounnas at SIBIA Neurosciences) using Aβ40 end specific monoclonal antibody. Note that at both the 48 and 72 time interval, LRP−/− cells transfected with APP751 secreted approximately 50-90% less AP than control LRP± cells transfected with APP751 (Panel E).

F: Inventors transfected the LRP-CT construct into CHO cells already expressing APP751. Note that in the presence of the LRP-CT construct Aβ secretion is dramatically inhibited.

FIG. 11a shows an increase in APPs secretion in LRP−/− cells.

FIG. 12a shows loss of APP CTF in LRP−/− cells expressing APP695.

FIG. 12b shows APP CTF and APPs reduced in LRP−/− cells, but can be restored with expression of LRP-CT.

FIG. 13 shows reduced APP internalization in LRP−/− cells, partially restored with LRP-CT.

FIG. 14 demonstrates delayed turnover of full length APP in LRP−/− cells by pulse chase analysis (upper panel). Increased turnover of APP CTF in LRP−/− cells by pulse chase analysis (bottom panel).

FIG. 17 demonstrates APP and LRP co-immunoprecipitations. In upper panel, note that APP complexes with LRP β-subunit as well as full length LRP (α+β) [right lane]. In bottom panel, APP complexes with LRP-CT.

FIG. 18 shows that Aβ secretion is not reduced in C99 APP construct mutated at the endocytic motif (Y/A)EN(P/A). The cDNAs were transiently transfected into HEK293 cells in duplicate and Aβ precipitated from medium.

FIG. 19 demonstrates co-fractionation of full length APP and APP CTFs with caveolin-1 in low density fractions of CHO cells transfected with APP751.

FIG. 20 demonstrates that transfection of LRP-CT in APP transfected CHO cells reduce Aβ secretion in proportion to level of expression. Upper panel, clone CT2 expresses LRP-CT several fold more than endogenous LRP β-subunit.

FIG. 21 shows N2a neuroblastoma cells stained for β-galactosidase 4 hours after Tat-β-galactosidase was added to the culture medium. Essentially all the cells are positive.

FIG. 22 depicts the expression of human PS1 in brain under Tet-regulation. In absence of tetracycline, there is abundant expression of the PS1 transgene as detected by an antibody specific for human PS1. For comparison, the level of PS1 is higher than a transgenic line under control of the PDGF promoter.

### DESCRIPTION OF THE PREFERRED EMBODIMENTS

### INTRODUCTION

The low density lipoprotein receptor-related protein (LRP) is a member of the LDL receptor family of endocytic receptors. Although structurally similar to other members of the LDL receptor gene family, LRP is considerably larger. LRP is a 600 kDa transmembrane glycoprotein that is cleaved in the trans-Golgi network by furin to generate a 515 kDa α- and an 85 kDa β-subunit (Herz et al., 1990). The subunits remain associated in a noncovalent fashion as they are routed to the cell surface (Herz et al., 1990; Willnow et al., 1996); from there LRP recycles between endosomal locations and the cell surface (Ward et al., 1989). More than 19 ligands have been reported to bind to the large 515 kDa N-terminal fragment of LRP, which can be divided into four ligand binding domains (Moestrup et al., 1993; Willnow et al., 1994; Holtzman et al., 1995; Buand Rennke, 1996; Horn et al., 1997), but not to the 85 kDa C-terminal β-subunit of LRP. While the LDL receptor appears to function exclusively in lipoprotein metabolism, increasing evidence suggests that LRP and the other members of the gene family have diverse biological roles, including signal transduction (Boucher et al., 2002; Loukinova et al. 2002) and neurotransmission (reviewed by Herz 2001).

LRP and several of its ligands: amyloid precursor protein (APP); apolipoprotein E (ApoE); and α-2-macroglobulin (α2M), all of which are found within senile plaques, have been genetically associated with AD (reviewed by Hyman et al., 2000). In addition, LRP has been shown to be a receptor for the Kunitz proteinase inhibitor (KPI)-containing isoform of the amyloid precursor protein (APP). As the KPI isoform is present either as a cell surface (APP) or secreted molecule (APPs), both, upon complexing with serum proteases, have been shown to interact with LRP (Kounnas et al., 1995; Knauer et al., 1996; Rebeck et al., 2001). LRP has been shown to mediate clearance of amyloid β-protein (Aβ)-α2M complexes in vitro and possibly facilitate the clearance of Aβ from brain in vivo (Kang et al., 2000; Narita et al., 1997; Shibata et al., 2000). Aβ derived from APP is the principal constituent of senile plaques deposited in brains of AD individuals, and is believed to hold a central position in AD pathogenesis (reviewed by De Strooper and Annaert, 2000).

Although the factors governing production and deposition of Aβ are not fully understood, it has been shown that APP can undergo at least two post-translational processing pathways. In one pathway, APP is cleaved within the Aβ region by a proteinase activity known as α-secretase, which recently has been determined to be a member of the ADAM family of metalloproteinases (reviewed by De Strooper and Annaert, 2000). This gives rise to the secreted form (APPs) and prevents generation of an intact Aβ polypeptide. The residual 10 kDa C-terminal APP fragment (CTF-α) remains within the plasma membrane and can be cleaved by γ-secretase activity to release a truncated Aβ peptide, termed p3.

Alternatively, APP can undergo proteolytic cleavage by β-secretase (BACE), followed by γ-secretase activity to generate Aβ (Vassar et al., 1999; Hussain et al., 1999). This is referred to as the amyloidogenic pathway of APP processing, which can take place intracellularly in the secretory compartments, or following internalization of cell surface APP in the endocytic pathway (Koo and Squazzo, 1994; Cook et al., 1997; Hartmann et al., 1997; Skovronsky et al., 1998). It has been suggested that the cytoplasmic YENPTY sequence mediates APP internalization into endosomes, and in turn, generation of the secreted pool of Aβ (Koo and Squazzo 1994; Perez et al., 1999).

In a recent study, LRP was directly linked to Aβ production in cells overexpressing APP751. When LRP binding was blocked by the 39 kDa receptor associated protein, RAP, or in cells deficient in LRP, Aβ levels were dramatically reduced (Ulery et al., 2000). This led the authors to propose that interaction between LRP and the KPI domain of APP at the cell surface modulates APP internalization and subsequent Aβ production. Although this attractive hypothesis is consistent with the current models of Aβ production, it implies that the neuron specific APP695 isoform, which lacks the KPI domain, is excluded from this interaction. This prediction would, at first approximation, be surprising since neuronal APP is believed to be the major source of Aβ in brain. In this study, Inventors tested this hypothesis and their results failed to confirm this proposed model. Inventors show in cells deficient in LRP that a number of steps in APP processing, trafficking, and turnover are indeed impaired in the absence of LRP. Surprisingly, these perturbations are present with both APP695 and APP751 isoforms. Moreover, these alterations can be restored when n an artificial LRP construct, which truncates the N-terminus of the 85 kDa β-subunit but retains the entire cytoplasmic domain, is expressed in the LRP deficient cells, indicating that interactions between the respective cytoplasmic regions play a major role in APP processing. Inventors further narrowed the essential region in the cytoplasmic tail to a small domain that encompasses the second NPXY motif in LRP. In view of the findings that a number of cytoplasmic proteins have been shown to interact with the cytoplasmic tail of both APP and LRP, Inventors propose a model by which APP processing is modulated by communication between the cytoplasmic domains of APP and LRP, possibly via the cytoplasmic adaptor proteins.

Hypothesis: LRP Interacts with APP Via the Cytosolic Domain to Regulate APP Processing.

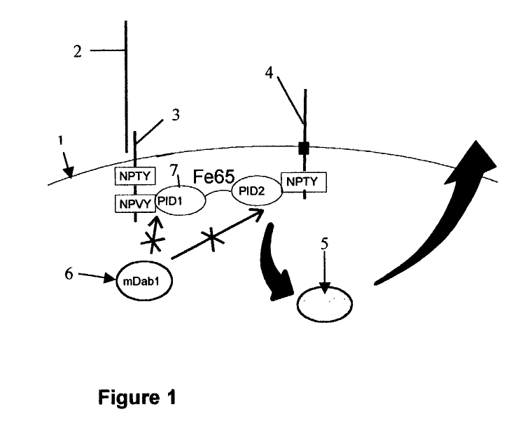

The mechanism put forth by Ulery and colleagues is attractive for a number of reasons. First, it links the recognized binding of APP to LRP to a known pathway of Aβ generation, i.e. the endocytic pathway. Second, it provides new insights into how APP processing can be regulated into the amyloidogenic and non-amyloidogenic pathways. However, this model also implies that the neuron-specific APP695 isoform, which and overexpression of these proteins, such as Fe65 and X11/Mint-1, have been shown to alter APPs secretion and Aβ generation, though not in any consistent fashion [Ando K, et al. J. Biol. Chem. 276:40353-40361, 2001, Borg J, et al. J. Biol. Chem. 273, No 24:14761-14766, 1998, Borg J P, et al. Mol. Cell Biol. 16:6229-6241, 1996, Fiore F, et al. J. Biol. Chem. 270:30853-30856, 1995, Guenette S Y, et al. J. Neurochem. 73:985-993, 1999, Sastre M, et al. J. Biol. Chem. 273, No 35:22351-22357, 1998, Sabo S, et al. J. Biol. Chem. 274, No. 12:7952-7957, 1999]. In this context, Trommsdorf and colleagues speculated that the respective cytoplasmic regions of LRP and APP communicate with each other via these adaptor molecules to influence APP trafficking, although no experimental data was presented [Trommsdorff M, et al. J. Biol. Chem. 273:33556-33560, 1999]. Consistent with this view, LRP, Fe65, and APP were found to colocalize with each other by FRET analysis [Kinoshita A, et al. J. Neurosci. 21:8354-8361, 2001]. Importantly, in Inventors preliminary studies described below, Inventors have obtained compelling evidence that the cytosolic tail of LRP is necessary and sufficient to modulates APP processing (Section C.4). Loss of LRP affects APP at multiple steps, much more so than was originally described by Ulery and colleagues. In fact, the perturbations in APP processing (APPs, APP C-terminal fragments (CTF), Aβ, APP turnover, APP internalization) are even more extensive than have been described in presenilin deficient cells. Significantly, these alterations in APP processing in LRP-deficient cells are essentially equivalent between APP751 and APP695 isoforms, i.e. APP isoform independent. Furthermore, perturbations in these APP processing steps can be restored when an artificial LRP construct consisting of the C-terminal 370 amino acids (a N-terminal truncated LRP β-subunit), lacking all the known ligand binding domains, is expressed in the LRP deficient cells. Finally, Inventors identified the cytoplasmic tail, and possibly the second of two NPXY motifs, is necessary and sufficient for the effects on APP processing. Taken together, these studies provided strong evidence that LRP may be at the center of a cellular pathway that mediates not only Aβ clearance but also Aβ production through it's regulation of APP processing (FIG. 1).

### MATERIALS AND METHODS

### EXAMPLE 1

Cell Lines and cDNA Constructs

Mouse fibroblasts deficient in LRP (LRP−/−; PEA 13) and corresponding LRP expressing control cells (LRP±; PEA 10) were obtained from American Type Culture Collection (Rockville, Md., USA) and cultured as described previously (Kang et al., 2000). LRP deficient CHO cell line (13-5-1), CHO cell line expressing a LRP trafficking mutant (14-2-1), and corresponding CHO K1 control cells (kind gift of Dr. S. Leppla) were grown in alpha DMEM supplemented with 10% fetal bovine serum (FitzGerald et al., 1995). Human APP695 and APP751 were inserted into the pBabepuro retroviral expression vector (Morgenstern and Land, 1990) and transferred into GP+E86 packaging cell line. Stable transformants were selected with puromycin (5 μg/ml). After infection with recombinant viruses, mouse fibroblasts were selected with puromycin (2.5 μg/ml)and analyzed after clonal selection for equal APP expression. The last 370 amino acids of the 601 amino acid LRP β-subunit, consisting of the transmembrane domain and the entire cytoplasmic region, and its corresponding signal peptide sequence (LRP-CT) were subcloned into the pBabehygro retroviral expression vector (Morgenstern and Land, 1990) and transferred into GP+E86 packaging cell line. Stable transformants were selected with hygromycin (2.5 μg/ml). After infection with recombinant viruses, LRP−/− mouse fibroblasts were selected with hygromycin (2.5 μg/ml) and analyzed (LRP−/−CT). Deletion constructs lacking amino acid residues 4469-4484 (LRP-CTΔ1) and amino acid residues 4486-4507 (LRP-CTΔ2) designed to exclude the two NPXY-motifs (residues 4470-4473 and 4504-4507), respectively, were generated by PCR. Retroviruses expressing the two deletion constructs were used to infect LRP−/− fibroblasts as before. After hygromycin selection, individual clones were selected for analysis based on comparable expression levels of LRP-CTΔ1 and LRP-CTΔ2. LRP deletion constructs lacking amino acid residues 4505-4507 (LRP-CTΔNPVY) or amino acid residues 45074510 (LRP-CTΔYATL) were generated by PCR were transiently transfected into CHO14-2-1 using Fugene6 (Roche).

### EXAMPLE 2

Antibodies

The polyclonal antiserum (1704) was generated by immunizing rabbits with a synthetic peptide corresponding to the last 15 amino acids of the cytoplasmic domain of human LRP coupled to keyhole limpet hemocyanin. Monoclonal antibodies 1G7, 5A3 and 26D6, which react with the ectodomain of APP, have been described previously (Koo et al., 1996; Kang et al. 2000). Polyclonal antibody CT15, which reacts with the cytoplasmic domain of APP and the polyclonal antiserum 863, which was raised against the mid-region of APP, has been described previously (Sisodia et al., 1993; Marquez-Sterling et al., 1997).

### EXAMPLE 3

Immunoprecipitation and Immunoblotting.

For detection of intracellular proteins, cell extracts were prepared using an NP40 lysis buffer. To detect APPs and secreted Aβ, media conditioned by the respective cell lines for 24 h or 72 h were collected for analyses. For APPs detection, conditioned media were immunoprecipitated using the monoclonal APP ectodomain antibodies 1G7 and 5A3. Extracts and immunoprecipitates were fractionated by SDS-PAGE in 4-12% tris-glycine gels. In all cases gel loading was normalized to total protein content in the cell extract or the corresponding cell extracts when medium samples were used. Western blotting was carried out with the indicated antibodies and detected by enhanced chemiluminescence (Pierce). Quantitation of the chemiluminescence signal was carried out with a CCD camera imaging system (GeneGnome, Syngene, Frederick, Md.).

### EXAMPLE 4

Ab ELISA Measurements.

Media from LRP−/−, LRP± LRP−/−CT fibroblasts was collected after an incubation period of 72 hours in serum free IS-CHO medium. Debris was removed by centrifugation (13,000 rpm for 20 min) and the supernatants were subjected to Aβ40 quantification using a standard sandwich ELISA described previously (Kang et al., 2000).

### EXAMPLE 5

Metabolic Labeling.

Confluent cultures of APP751 transfected LRP± and LRP−/− fibroblasts were incubated in methionine-free Dulbecco's modified Eagle's medium supplemented with 150 μCi/ml [35S] methionine/cysteine for 15 min. Cells were lysed immediately (time 0) or chased for 1, 2, or 4 hours to determine the turnover of APP. APP-CTF turnover was determined by incubation with 150 μCi/ml [35S] methionine/cysteine for 1 hour. Cells were lysed immediately (time 0) or chased for 3, 6, or 18 hours. For these studies, APP was immunoprecipitated with polyclonal antibodies CT15. The immunoprecipitates were fractionated by SDS-PAGE (6% Tris-glycine gels for full length APP and 4-12% Tris-Tricine gels for APP-CTF) and exposed to either film or to phosphor imaging for quantitation.

### EXAMPLE 6

Surface Labeling and Uptake Assay.

To measure APP surface expression or internalization of surface APP, iodinated 1G7 monoclonal antibody was added to the cells in triplicate exactly as described previously (Koo et al., 1996). For the measurement of cell surface APP, the iodinated antibody was added to APP transfected LRP± and LRP−/− fibroblasts at 4° C. for 30 min followed by extensive washes. Specific binding was calculated after subtraction of the radioactivity obtained from parallel cultures of untransfected LRP± and LRP−/− fibroblasts, respectively. The radioactivity was then normalized to the rate of production of APP, which was determined in parallel cultures. The latter cells were metabolically labeled as described before and immunoprecipitated with polyclonal APP antibody to the C-terminus (CT15). Immunoprecipitates were subjected to SDS-PAGE, gels were dried and the APP signal was quantified by phosphor imaging.

To measure internalization of cell surface APP, iodinated 1G7 antibody was added to confluent cultures of LRP±, LRP−/−, or LRP−/−CT fibroblasts stably transfected with APP695 or APP751 at 37° C. for 30 min. After incubation, cells were rapidly chilled on ice, and the reaction was quenched by the addition of ice-cold binding medium. Chilled cells were extensively washed and remaining surface antibody was detached by two acid washes. The cells were then lysed and collected for analysis. Acid-labile and acid-resistant radioactivity represents the surface and internalized pools of APP, respectively. Specific binding was determined after subtraction of the radioactive counts obtained from parallel cultures of untransfected LRP± and LRP−/− fibroblasts. The ratio of acid-resistant to acid-labile counts therefore provided a measure of the internalized versus cell surface pools of APP. All experiments in this study were repeated one to three times. Results are presented as either the averages of all experiments ±SEM or a representative experiment ±SEM.

### EXAMPLE 7

Cell Surface Biotinylation.

Cell surface proteins were biotinylated with 0.5 mg/ml EZ-Link Sulfo-NHS-biotin (Pierce) in 10 mM borate buffer, pH 9.0, for 30 min at 4° C. Following washes with PBS containing 50 mM NH4Cl to quench any unconjugated biotin, the cells were lysed in NP40 lysis buffer and incubated overnight with streptavidin-agarose beads. Bound proteins were eluted from the beads by boiling the samples for 5 min in sample buffer. Extracts were fractionated by SDS-PAGE in 4-12% tris-glycine gels followed by Western blotting with the anti C-terminal LRP antibody 1704.

### RESULTS

### EXAMPLE 8

APP Processing is Altered in the Absence of LRP.

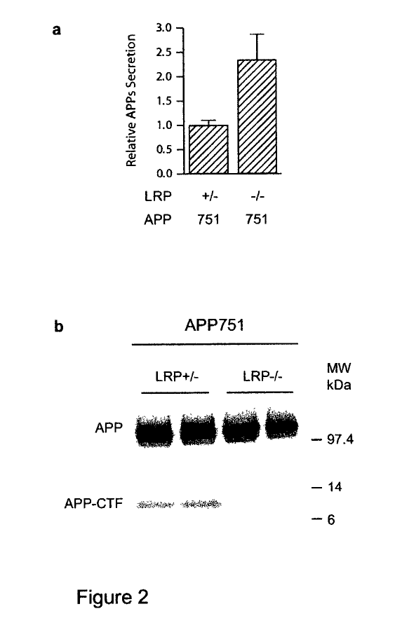

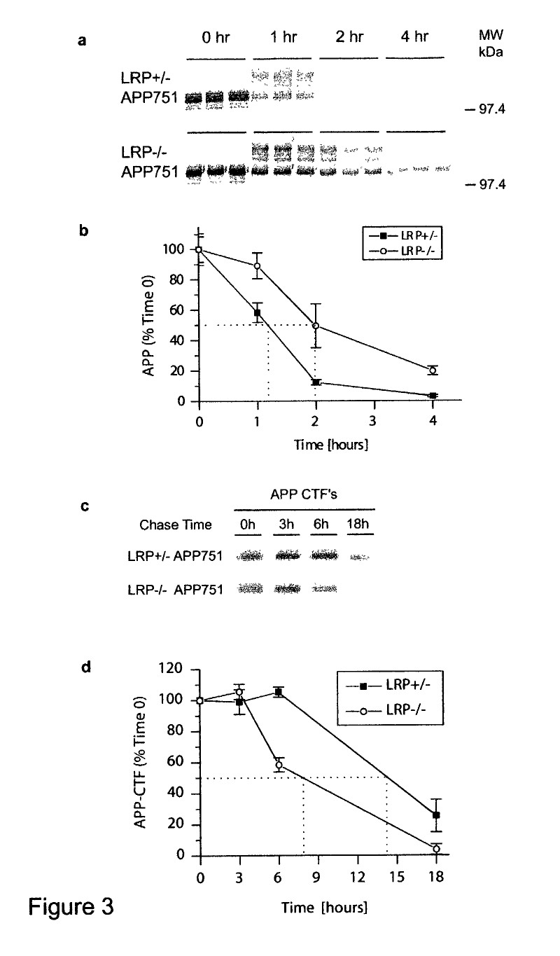

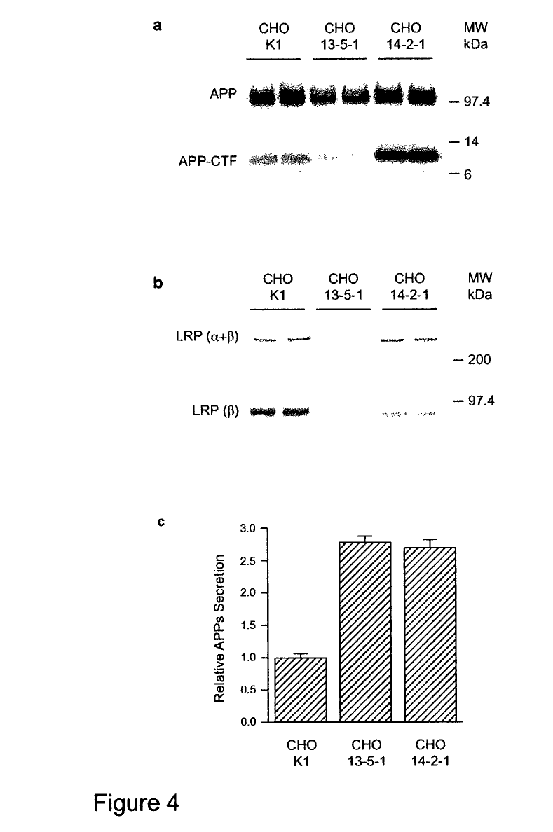

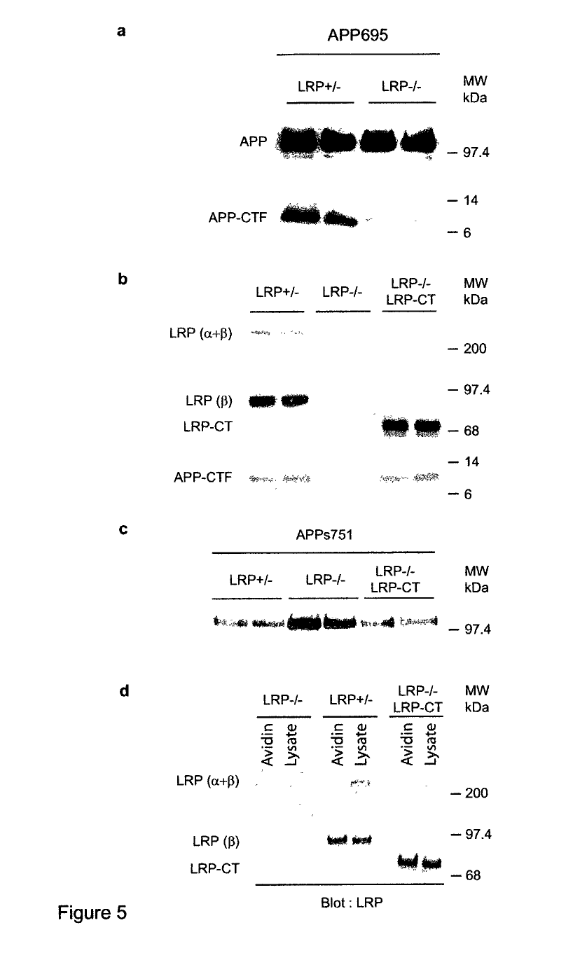

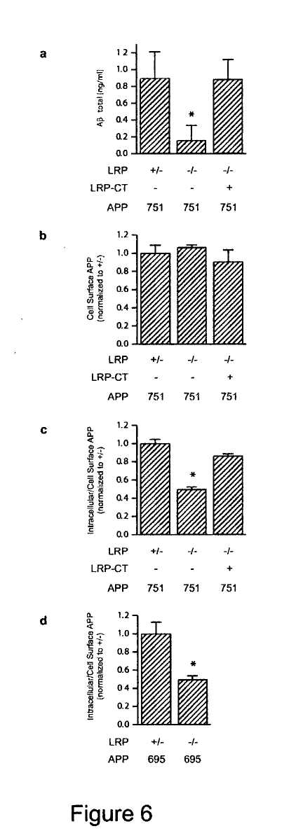

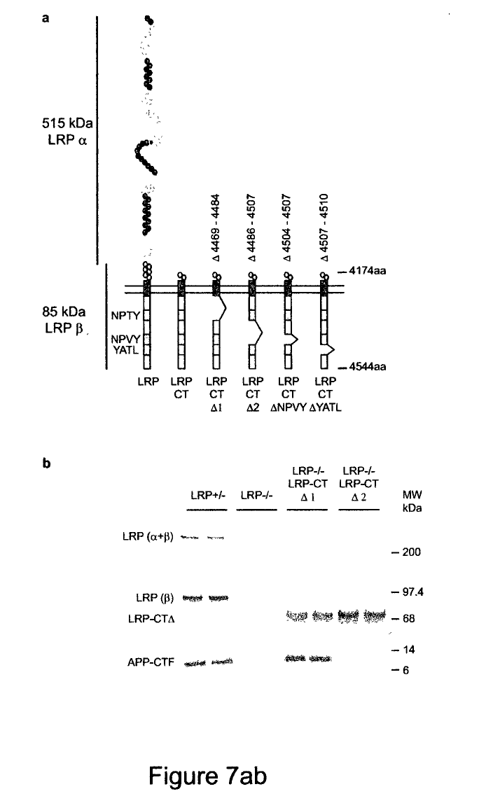

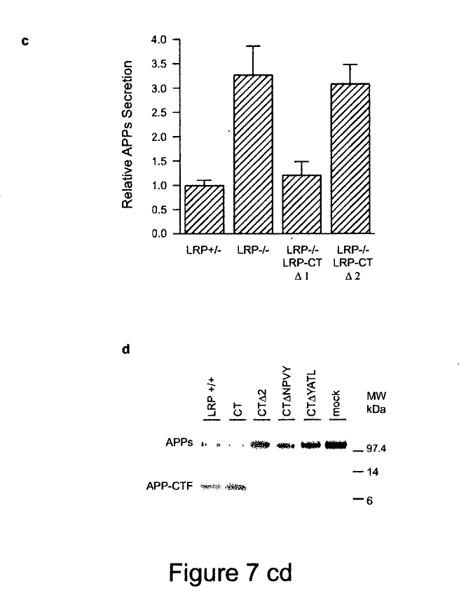

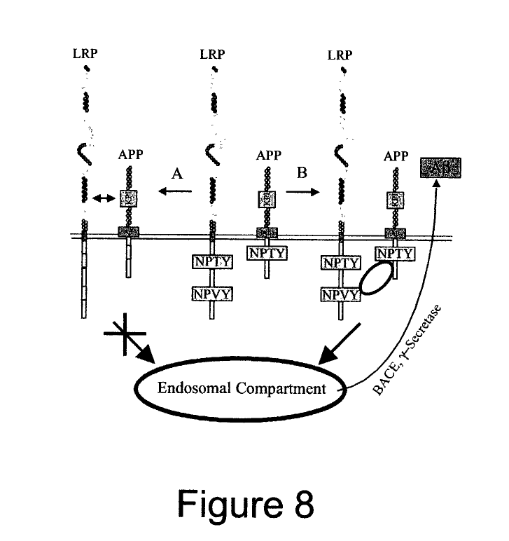

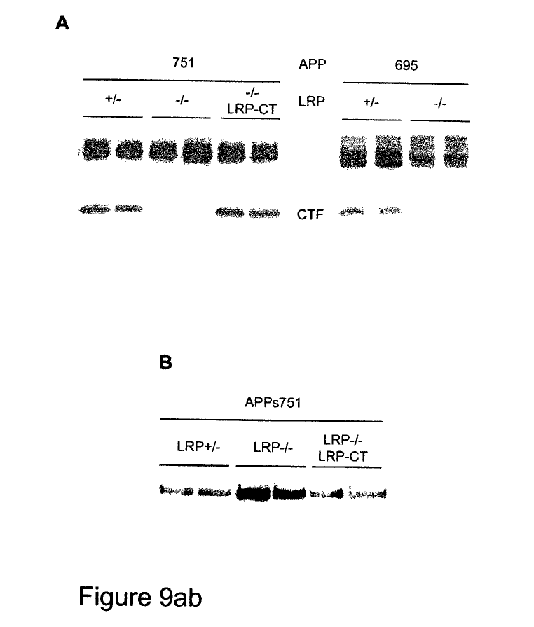

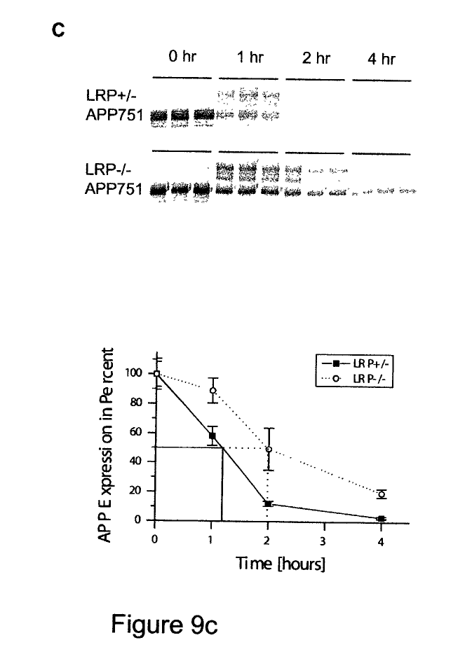

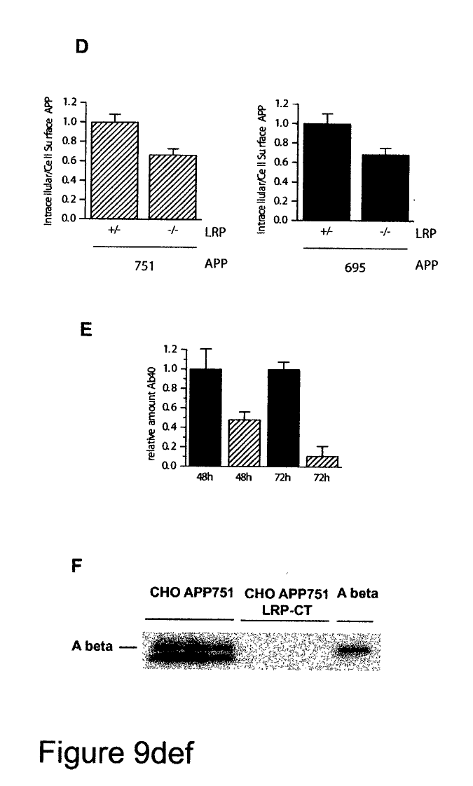

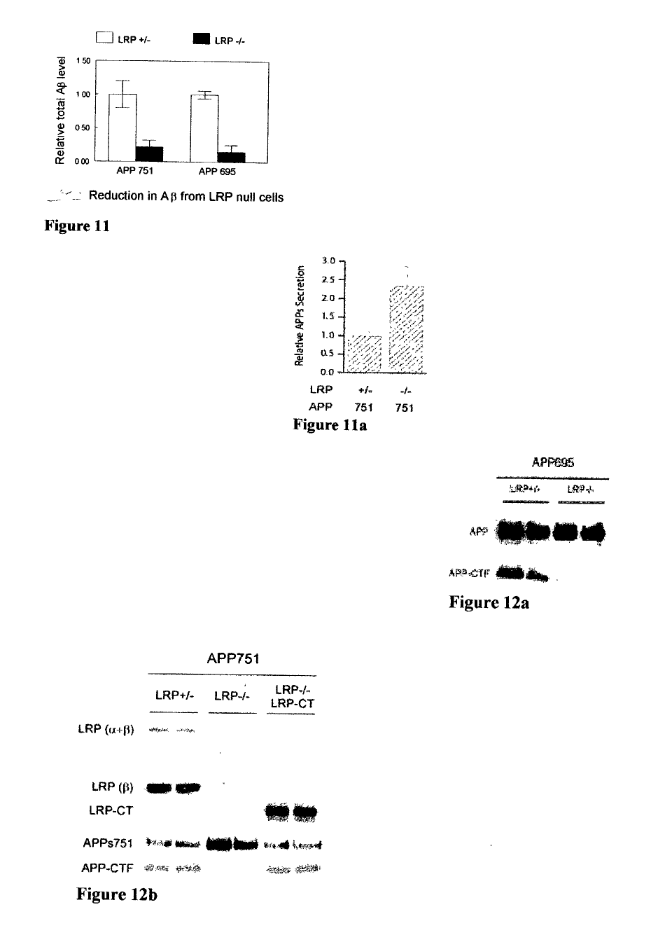

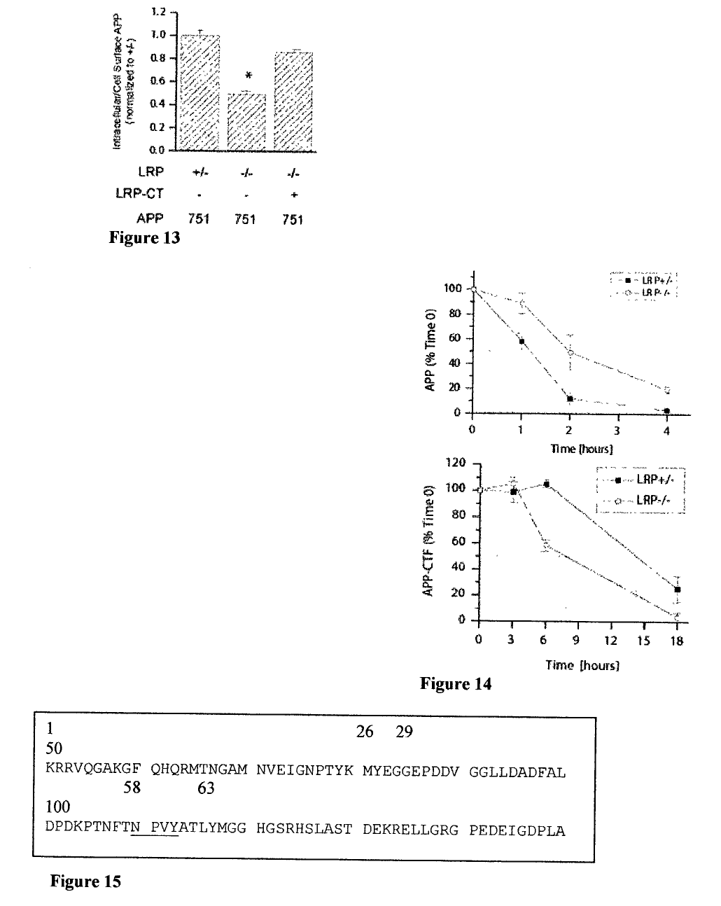

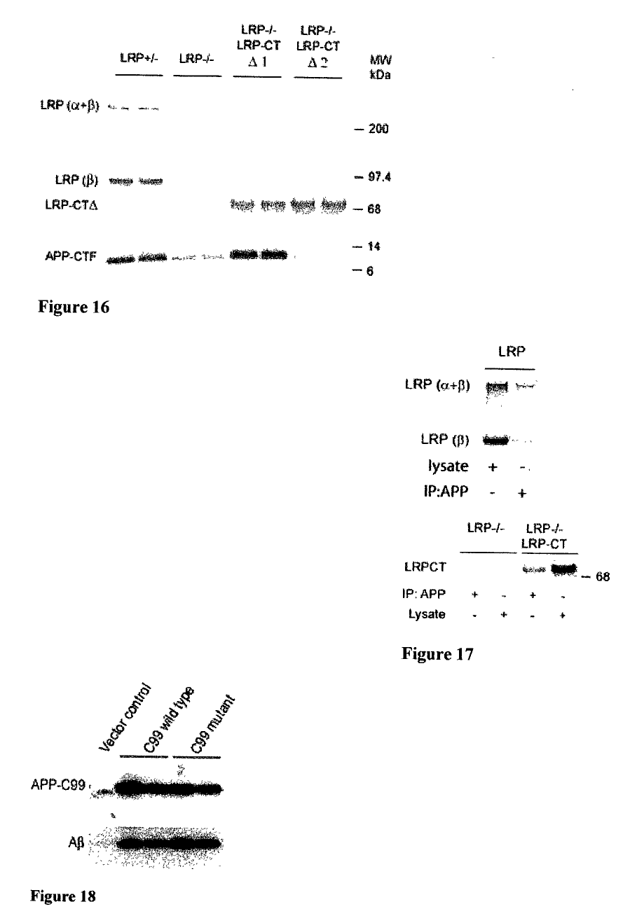

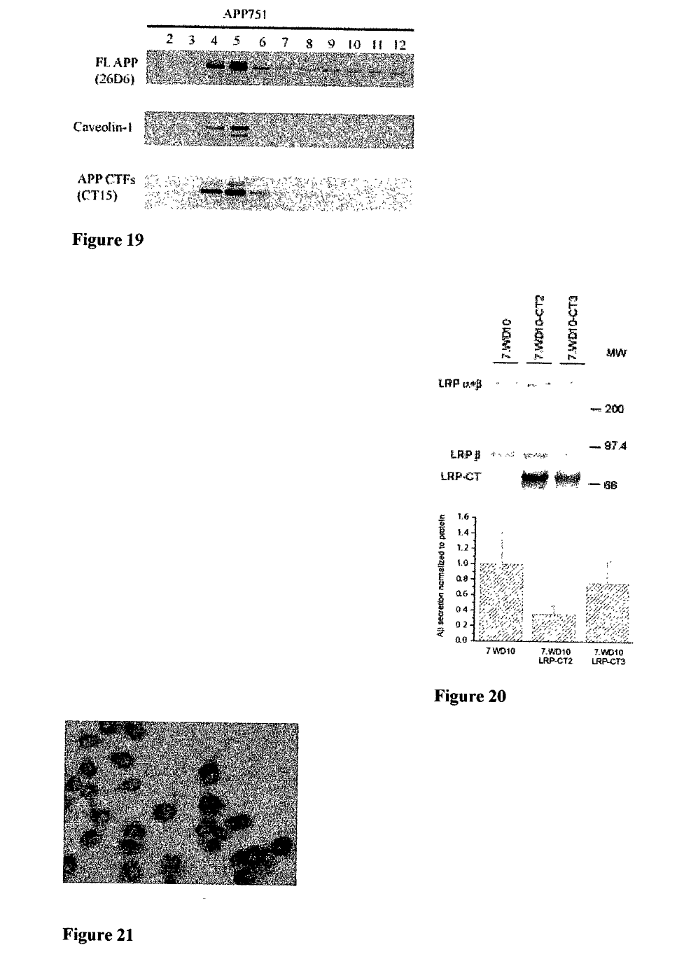

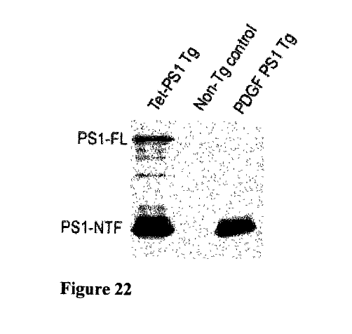

To determine whether the processing of APP is affected in the absence of LRP, APP751 was first introduced into mouse fibroblasts deficient in LRP (LRP−/−) and corresponding control (LRP±) cells by retroviral mediated infection. Single clones of comparable expression were chosen for analyses. When the cell supernatants were normalized to APP expression, the level of APPs in LRP−/− cells transfected with APP751 was 2.5 fold higher than LRP± cells (FIG. 2a), consistent with the recently reported results (Ulery et al., 2000).

We next assayed for APP C-terminal fragment (CTF) in LRP deficient cells because APPs secretion and the generation of CTFs are frequently correlated with each other. Although the expression of APP in LRP−/− and control LRP± cells was comparable (FIG. 2b, top), the levels of the α-secretase cleaved CTF (CTF-α) were significantly decreased in LRP−/− cells (between 4-6 fold; average 5.2 fold ±1.8 SD n=4), as compared to controls (FIG. 2b, bottom).

The marked reduction in the amount of APP-CTF in LRP deficient cells was unexpected. Accordingly, Inventors next examined the turnover of full length APP by pulse chase analysis in LRP± and LRP−/− fibroblasts stably transfected with APP751 to determine if instability of APP can account for loss of CTFs. Unexpectedly, the turnover rate of APP in LRP−/− fibroblasts was dramatically slowed rather than increased as compared to control fibroblasts, indicating that LRP normally facilitates the rapid degradation of APP. Even after 4 hr chase period, APP can still be detected in LRP−/− fibroblasts at a time when APP in control cells was virtually all degraded (FIG. 3a). The normal short half-life of APP (˜70 min) was almost doubled (˜120 min) in LRP deficient cells (FIG. 3b).

The reduced turnover of full length APP in LRP deficient cells is surprising because increased stability of APP should intuitively have resulted in more, not less, APP-CTF. Therefore, the turnover of APP-CTF itself was next analyzed by pulse chase paradigm. The cells were labeled for 1 hour, a duration that was found to result incomparable levels of CTFs in both cell LRP−/− and LRP± cells (FIG. 3c), indicating that generation of CTF was not impaired. After the initial 3-hour chase period, APP-CTF levels in LRP± and LRP−/− cells remained similar. At later time points however, APPCTFs in LRP−/− were considerably more unstable. After 18 hr chase period when APPCTFs were still present in LRP± fibroblasts, APP-CTFs in LRP−/− fibroblasts were almost completely degraded (FIG. 3c). The turnover rate of APP-CTFs in LRP−/− cells was approximately doubled (FIG. 3d). These results therefore indicate that the low levels of APP-CTF in LRP−/− cells are due in part from reduced stability of the fragments in the absence of LRP. The above studies were performed in mouse fibroblasts overexpressing human APP. To exclude false positive results caused by overexpression, Inventors next analyzed untransfected CHO cells deficient in LRP (13-5-1) (FitzGerald et al., 1995). Confirming the results in mouse fibroblasts overexpressing APP, the levels of CTFs derived from endogenous APP, predominantly APP751, were consistently decreased in LRP deficient CHO 13-5-1 cells as compared to controls (FIG. 4a). In addition, since generation of APP-CTFs can occur both in the secretory and in the late endosomal-lysosomal compartments, a CHO cell line expressing a mutant form of LRP (14-2-1) was analyzed to determine where the turnover of APP-CTF was perturbed. Specifically, Inventors asked whether retention of LRP in the ER and Golgi complex might restore the increased instability of APP-CTF seen in LRP deficient cells. The CHO cell line, 14-2-1, expresses a mutant LRP molecule that is defective in Golgi to plasma membrane trafficking, resulting in retention of LRP in the ER and Golgi compartments (FitzGerald et al., 1995). In these cells, the abnormal trafficking of LRP is reflected in the increased ratio of full-length (a+βsubunit) LRP to light chain (β-subunit) LRP because the mutant LRP is retained in the early compartment, thus reducing the level of furin cleaved β-subunit (FIG. 4b top). Interestingly, in CHO 14-2-1 cells, not only is the endogenous APP-CTF level not reduced as in CHO 13-5-1 LRP deficient cells, the level even exceeds that seen in control CHO K1 cells while the full-length APP remained unchanged (FIG. 4a). These results indicate that LRP in the early compartments influences specifically the stability of APP-CTFs. However, secretion of APPs (FIG. 4c) in 14-2-1 cells are altered similar to cells lacking LRP expression, indicating that post-Golgi APP trafficking steps are highly dependent on LRP (see below).

### EXAMPLE 9

Processing of APP695 is Altered by an LRP Knock-Out.

The above results are consistent with the notion that LRP influences many aspects of APP processing, indeed much more than the changes in APPs and Aβ that were reported recently (Ulery et al., 2000). Inventors next wanted to determine whether the KPI domain in APP is solely responsible for these changes in APP processing. For this analysis, single clones of LRP−/− and LRP± fibroblasts expressing comparable levels of APP695 were isolated. Surprisingly, the levels of the APP-CTFs derived from APP695were also significantly reduced in LRP−/− cells as compared to controls (FIG. 5a). This suggested that processing of APP is not regulated exclusively through interaction of its KPI-domain with LRP since the APP695 isoform lacking this domain also demonstrate LRP dependent regulation of CTF turnover.

The finding from APP695 expressing LRP−/− cells suggested that although the interaction between LRP and APP is higher with KPI containing APP isoform (Rebeck et al., 2001), the KPI domain is not essential for LRP dependent aspects of APP processing. Rather, it suggests that the respective C-termini of APP and LRP play functionally important roles in APP trafficking, a concept first proposed by Trommsdorf and colleagues. To test this hypothesis, a truncated LRP construct consisting of the Cterminal 370 amino acids of LRP (LRP-CT) was engineered, and stably introduced into LRP−/− cells expressing APP751. This follows the approach taken by several laboratories that used mini-LRP receptors to delineate a number of functional ligand binding s domains of LRP (Herz et al., 1990; Li et al., 2000). However, the LRP-CT construct used in this study is a N-terminal deleted β-subunit, thus lacking all four functional ligand-binding domains. Interestingly, when LRP-CT construct was introduced in LRP deficient cells expressing APP751 (FIG. 5b, top), the diminished levels of APP-CTFs were restored to levels similar to LRP± cells (FIG. 5b, bottom). To further confirm this hypothesis, were-examined the LRP dependent changes in APPs secretion first noted in this study** (FIG. 2a). Consistent with the APP-CTF result, introduction of LRP-CT construct into LRP−/− cells also restored the levels of APPs back to the levels seen in APP751 overexpressing cells (FIG. 5c). Finally, Inventors used surface biotinylation to ascertain that LRP-CT reaches the cell surface (FIG. 5d), an anticipated finding since LRP-CT is able to restore the reduction in APP-CTF levels in LRP−/− cells. Taken together, these experiments suggest that the β-subunit, possibly only the cytoplasmic domain of LRP, play an important role in regulating APP processing.

### EXAMPLE 10

Ab Levels in LRP±, LRP−/−, and LRP-CT Fibroblasts.

As described above, it was recently reported that loss of LRP is associated with a marked reduction in Aβ levels, which was attributed to loss of the normal interaction between LRP and the KPI domain of APP (Ulery et al., 2000). In view of the changes in APP processing that are independent of the KPI-isoform, Inventors next examined the levels of Aβ in LRP deficient fibroblasts. In LRP−/− cells transfected with APP751, the level of Aβ in medium was reduced to approximately 20% of control LRP± cells transfected with APP751 after 72 hours collection. However, expression of LRP-CT construct in LRP−/− cells resulted in a dramatic increase in the amount of Aβ in medium (FIG. 6a). Indeed, similar to APPs (FIG. 5c), Aβ from LRP−/− cells expressing LRP-CT was restored to control level present in LRP± control cells.

### EXAMPLE 11

Surface Expression and Internalization of APP in LRP± LRP−/− and LRP−/− LRP-CT Fibroblasts.

The internalization of cell surface APP is believed to represent a major pathway for generation and subsequent release of Aβ into the extracellular space. Inventors therefore examined if transport of APP to the cell surface and its subsequent endocytosis are affected by the C-terminus of LRP. Iodinated APP monoclonal antibody, 1G7, was used to determine APP surface expression in LRP± and LRP−/− fibroblasts stably expressing APP751. Using this assay, the levels of APP at the cell surface in LRP± and LRP−/− cells were unchanged (FIG. 6b). Similarly, the expression of LRP-CT in LRP deficient cells did not alter the amount of cell surface APP (FIG. 6b). Since the level of cell surface APP is not grossly impaired in the absence of LRP, this result suggests that the reduction in Aβ production in LRP−/− cells is not secondary to a gross defect in transport of APP to the cell surface. In LRP−/− and LRP± cells stably transfected with APP695 or APP751 constructs, APP endocytosis was measured using iodinated APP antibody. With this established assay, the internalization of APP was reduced by 50% in LRP−/− cells as compared to LRP± control cells. Significantly, this reduction in APP internalization was seen in both APP695 and APP751 expressing cells (FIGS. 6c and d). Inventors then focused on APP751 expressing LRP−/− cells and asked whether this defect in APP internalization is similarly related to the absence of the LRP C-terminus. Indeed, consistent with all the changes noted above, the impairment in APP internalization was largely restored to control level following introduction of LRP-CT construct in LRP−/− cells (FIG. 6c).

### EXAMPLE 12

The C-Terminus of LRP Modulates APP Processing.

In this study, each LRP-dependent step in APP processing perturbed in the absence of LRP can be restored by the expression of the C-terminus of LRP (LRP-CT), and where tested, both APP695 and APP751 isoforms showed the same defects when LRP is absent. These results provided strong evidence that is the LRP C-terminal fragment, and potentially only the cytoplasmic domain, is important in the regulation of multiple steps in APP processing. A number of important physiological roles have been attributed to the cytoplasmic tail of LRP, among which are two NPXY motifs that have been shown to interact with a family of cytoplasmic adaptor proteins. However, the N-terminal truncated LRP-CT construct still contains about 230 aa in the extracellular region where interaction with APP can theoretically take place, even though it does not contain any known ligand-binding domain. Therefore, to clarify which region in the C-terminus of LRP is involved in the modulation of APP processing, two LRP-CT mutants each deleting the respective NPXY motif were engineered. The two constructs, LRP-CTΔ1 and LRP-CTΔ2, deleting approximately 20 amino acids each, were stably transfected into LRP−/− cells expressing APP751. Single clones of comparable expression were isolated for analysis FIG. 7b). As before, loss of LRP is associated with a large decrease in the levels of APP-CTFs. Interestingly, LRP-CTΔ1, but not LRP-CTΔ2, restored APP-CTFs to control levels (FIG. 7b, bottom). Similarly, introduction of LRP-CTΔ1 but not the LRPCTΔ2 construct into LRP−/− cells also restored the levels of APPs to the levels seen in LRP± control cells (FIG. 7c). These results indicate that the responsible regulatory sequences are contained within LRP-CTΔ2, or between amino acids 4486-4507. This region contains the tetrapeptide NPVY motif, or the second of the two NPXY motifs in the cytoplasmic tail (FIG. 7a). Thus, this experiment established that it is specifically the cytoplasmic domain of APP that is critical for modulating APP trafficking. To narrow down the sequence within the LRP C-terminus responsible for these effects, Inventors engineered two additional deletion constructs lacking either the tetrapeptide NPVY domain (LRP-CTΔNPVY) or the adjacent tetrapeptide YATL domain (LRP-CTΔYATL). Transfection of APP695 and the respective LRP-CT constructs into LRP deficient CHO13-5-1 cells revealed that, unlike LRP-CT, neither the LRP-CTΔNPVY nor the LRPCTΔYATL was able to restore the levels of APP-CTF or secreted APPs to that seen in wild type LRP+/+ control cells (FIG. 7d). This suggested that tyrosine at position 4507 contained in of both deletion constructs is the critical residue.

### DISCUSSION

The view that lipoprotein receptors function predominantly if not exclusively in lipid transport has been challenged by recent findings that several members of the family which are receptors for apolipoprotein E function in development and signaling processes (reviewed by Herz and Buffer 2000; Boucher et. al., 2002; Loukinova et. al., 2002; Meet. al., 2002). In addition, a number of cytoplasmic adaptor and scaffold proteins bind to the cytoplasmic domain of LRP, although specific functions related to these interactions remain unclear. Therefore, the current study was designed to test the hypothesis that the regulation of APP processing by LRP occurs via the KPI domain and if not, test the hypothesis that this effect is mediated by the cytoplasmic domain of LRP. Inventors' studies showed that LRP modulates multiple steps in APP processing and trafficking, including the turnover of both full length and C-terminal fragments of APP, secretion of APPs, internalization of cell surface APP, and the gene ration and release of Aβ peptide. Significantly, because the processing of both APP695 and APP751 were altered in the absence of LRP, these changes must be independent of the KPI isoform of APP. This then led Inventors to demonstrate the cytoplasmic domain of LRP, specifically the amino acid residues around the second NPXY motif, encompassing a seven amino acid region (NPVYATL), is critical for the regulation of APP processing. Taken together, Inventors propose a model for LRP regulation of APP processing in which interaction between the cytoplasmic rather then the extracellular domains of LRP and APP are responsible for changes in APP processing and Aβ generation (FIG. 7).

In addition to the changes in the levels of APPs and Aβ noted previously, (Ulery al., 2000), Inventors' study showed that the absence of LRP produced two unanticipated effects on the turnover of APP. Specifically, the half-life of full length of APP was increased where the half-life of APP-CTFs was reduced. However, the slower turnover of full length APP and its reduced internalization in the absence of LRP does not account for the decreased stability in APP-CTFs. Inventors hypothesize that this change in APP-CTF stability is related to LRP's effects in the ER and intermediate compartments. This is because the level of APP-CTF was markedly increased in the CHO 14-2-1 cell line expressing a LRP trafficking mutant. LRP is not absent in this cell line but rather does not traffic to the cell surface as it is retained intracellularly. Thus, the elevation of APPCTFs in CHO 14-2-1 cells indicate an important interaction that influences the stability of APP-CTFs in these early compartments and prevents the C-terminal fragments from rapid degradation. This interpretation is consistent with not only the evidence that the ER and intermediate compartments are major sources of APP-CTFs (Annaert et al. 1999; Yan et al., 2001), it is also in agreement with the finding that APP associates with endogenous full length (α+β subunits) LRP (Data not shown), thereby placing the interaction in the same intracellular compartments. Taken together, Inventors hypothesize that the interaction between LRP and APP influences the turnover of APP-CTFs after their formation in the early secretory compartments.

In this study, Inventors provided evidence that LRP's effects on APP processing is mediated through the LRP cytoplasmic domain and that this interaction occurs early in the secretory compartments. By introducing various truncated LRP constructs into LRP-deficient cells, Inventors' studies showed that the distal C-terminus in the LRP cytoplasmic region is the major determinant of these effects. In particular, the LRP-CT deletion constructs (Δ1 and Δ2) implicated the second of the two NPXY motifs as important in this interaction. This notion was confirmed when two smaller deletion constructs: LRPCTΔNPVY (deleting residues 4504-4507) and LRP-CTΔYATL (deleting residues 4507-4510), were both unable to rescue the defects in APP processing in LRP deficient cells. Inventors postulate the tyrosine at position 4507 is the critical residue as it is common to both deletion constructs. Since LRP complexes with APP (Rebeck et al., 2001) but probably not directly, Inventors propose that this interaction specifically modulates APP processing. As LRP affects not only trafficking but also the stability of full length APP and the C-terminal fragments, Inventors hypothesize that these effects must be mediated in multiple intracellular compartments.

In the absence of LRP, Aβ level is markedly diminished, confirming an earlier report (Ulery et al., 2000). This decrease in Aβ is also correlated with a reduction in APP internalization from the cell surface. The impairment in APP internalization is in agreement with the hypothesis that endocytic processing is a major contributor to Aβ release. Similarly, the increase in APPs secretion is consistent with previous reports showing that loss of the APP endocytic signal elevates the levels of APPs in medium (Koo et al., 1996). In this regard, it is interesting that the NPVY-YATL motif in LRP implicated in Inventors' studies overlap with internalization signal in LRP. Although LRP contains two NPXY and two dileucine motifs that are consensus signals for endocytosis, Li and colleagues recently reported that the -YATL-tetrapeptide domain overlapping with second NPXY motif in the cytoplasmic tail of LRP is a major determinant in LRP internalization (Li et al., 2000). Since the region deleted in the LRP-CTΔ2, LRP-CTΔNPVY and LRP-CTΔYATL constructs include the tyrosine in the second NPXY motif (4486-4507), internalization of the truncated LRP molecule is likely impaired. It has been proposed that the LRP dependent changes are mediated via cytoplasmic adaptor proteins, which is capable of interacting with both APP and LRP via its multiple binding domains (Trommsdorf et al., 1998). Whether LRP and APP internalization is coordinately regulated through direct interaction or indirectly through adaptor proteins to regulate Aβ production is an intriguing possibility that awaits further study.

In summary, Inventors' results differed significantly from the proposed model in which LRP modulates APP processing via interaction in the respective extracellular domains, in particular, the KPI domain of APP (Ulery et al., 2000). Previous studies have convincingly demonstrated that the interaction of KPI isoform of APP with LRP is significantly greater than the non-KPI containing APP695 isoform, especially in complexes containing proteinases (Kounnas et al., 1995; Knauer et al., 1996; Rebeck et al., 2001; Kinoshita et al. 2001). The results reported in this study do not argue against this association. Rather, Inventors' findings suggest that the functional consequences of the interaction between LRP and the KPI isoform of APP are distinct from the APP trafficking steps examined in this study, namely, APP turnover, APPs secretion, APP internalization and Aβ production. Lastly, as members of the LDL receptor gene family, including LRP, are implicated in neurodevelopment, it is possible that the internalization of LRP and its binding partners might be important for transducing these extracellular signals (Trommsdorf et al., 1998; Hiesberger et al., 1999). Coincidently, APP has recently been shown to play a role in gene transcription similar to the Notch receptor (Cao and Sudhof 2001). In the latter situation, the cytoplasmic domain of APP together with Fe65 and Tip60 are required for a multimeric complex to translocate into the nucleus to affect transcription by a heterologous signaling system. Thus, LRP and APP, together with the cytoplasmic proteins, may indeed function beyond trafficking events and affect the transduction of cellular signals. If so, LRP may play a greater and varied role in Alzheimer's disease pathogenesis than has been suspected.

Overview

The focus of this disclosure is on the mechanisms of amyloid β-protein (Aβ) production from the amyloid precursor protein (APP). Increasing evidence suggests that the formation and deposition of Aβ in brain may be a very early and potentially critical event in the development of Alzheimer's disease (AD) pathology. The cellular pathways that lead to the production of Aβ are being defined, pointing to multiple intracellular sites where Aβ can be generated. Inventors have previously proposed that processing of APP in the endocytic pathway is a major pathway for the production and release of Aβ from wild type APP. Whereas the mechanisms of Aβ generation are becoming elucidated, how Aβ production is regulated within the cell is poorly understood. Inventors attention in the upcoming period shift to the role of the low density lipoprotein receptor-related protein (LRP) on APP processing and Aβ production.

LRP is a large type I membrane protein that functions as a multi-functional receptor with multiple roles in both ligand uptake and signal transduction. In regards to AD, LRP has been linked genetically to AD, is a receptor for secreted APP (APPs), is complexed to APP, and many of its ligands are found within senile plaques in brains of AD individuals. Significantly, Inventors and others have shown that LRP mediates clearance of A□ complexes, hence, a putative degradation pathway for A□. Surprisingly, Inventors recently found that loss of LRP results in a wide range of perturbations in APP processing, including diminished APP turnover, loss of APP C-terminal fragments, impaired APP internalization, increased APPs secretion, and a reduction in A□ production. These changes encompass a range of defects in APP processing that have previously not been attributed to an APP associated protein. The extensive changes lead Inventors to hypothesize that LRP regulates APP processing at multiple cellular compartments. Further studies suggest that a small region within the cytoplasmic tail of LRP is necessary and sufficient to mediate these effects. Thus, in it's receptor function, LRP is capable of removing extracellular A□ when complexed to an LRP ligand. At the same time, LRP interacts with APP in ways that dramatically alter the biology of APP. Inventors therefore propose that LRP has multiple and important roles in regulating APP processing and A□ production.

This invention uses established tissue culture and animal models to address the important regulatory mechanisms that affect A□ production and release. The working hypothesis that guides this study states that the interaction of APP and LRP via the respective cytosolic regions, either directly or indirectly, represents an important mechanism by which A□ generation is regulated. Results from the hereinbelow studies provide significant new insights into the pathways that regulate APP processing and trafficking, and in so doing, affect the generation of Aβ from neurons and non-neuronal cells. Moreover, the studies provide novel approaches by which A□ production can be targeted without inhibiting □-secretase activity indiscriminately.

PS1 is Localized to Early Secretory Compartments and Aβ can be Generated from the Golgi Compartment.

To understand the compartments where PS is localized and where □-secretase activity occurs, Inventors carried out a subcellular localization study of PS1 in transfected cells. Using subcellular fractionation, Inventors showed that full length PS1 is present principally in the ER compartment whereas the stable N- and C-terminal PSI fragments transit to the Golgi compartments (Borg J P, et al. Mol. Cell Biol. 16:6229-6241, 1996). Inventors' study did not address the issue of lipid rafts as those results had not yet been reported and Inventors' fractionation protocol would not have isolated the cholesterol rich microdomains. Nonetheless, Inventors do recognize that some laboratories have reported the presence of not only PS, but also APP, in these rafts. Consistent with the finding that PS is primarily in ER and Golgi fractions, Inventors subsequently showed that the accumulation of APP CTFs in PS1 deficient cells takes places in the same fractions (Bu G and Rennke S. J. Biol. Chem. 271:22218-22224, 1996). Interestingly, when these fractions were analyzed for Aβ levels, Aβ42/Aβ40 ratios were higher in Golgi fractions in cells expressing FAD-associated PS1 mutations. Inventors interpret these results to indicate that PS1 acts primarily in the Golgi compartment and that a portion of APP internalized from the cell surface transits to the TGN for □-secretase cleavage (Bu G and Rennke S. J. Biol. Chem. 271:22218-22224, 1996).

PS1 has Multiple Activities and the Hydrophilic Loop is not Required for APP γ-Secretase Activity.

In both PS1 and PS2, a large hydrophilic cytosolic domain contains sites for phosphorylation, caspase cleavage, endoproteolysis, and sequences that interact with a number of proteins. Inventors showed that the PS1 loop interacts with β-catenin, a central intermediate in the Wnt signaling pathway [Kang D E, et al. J. Neurosci. 19:4229-4237, 1999]. To determine if the hydrophilic domain is necessary for either PS endoproteolysis or Aβ42 production, a series of deletion mutants were generated and analyzed. Inventors' collaborative studies showed that PS has separate effects related to APP γ-secretase cleavage and the modulation of β-catenin turnover. These are separate activities that can be mapped to different domains. Whereas the hydrophilic loop domain is dispensable for PS endoproteolysis and APP γ-secretase activity (Chow N W, et al. J. Biol. Chem. 271:11339-11346, 1996), the loop is however required for facilitating PS1 turnover (Bu G and Rennke S. J. Biol. Chem. 271:22218-22224, 1996).

### EXAMPLE 13

APP V717F Mutation does not Increase Intracellular A□42 Levels

BACKGROUND: While the mutations at codon 717 were the first to show a selective increase in the levels of Aβ42, the mechanism by which this occurs is unknown. Inventors studied this question by examining the intracellular levels of Aβ42 from cells expressing the V717F mutation.

RESULTS: It has been reported that PS mutations increase not only the ratios of Aβ42/Aβ40 in extracellular fluids (plasma and culture medium) but also intracellularly. Inventors therefore examined Inventors' CHO cells that overexpress various APP mutations or PS1 mutations. Whereas intracellular levels of Aβ42 are elevated in cells expressing the PS1 mutations, Inventors were unable to document any changes in cells expressing APP V717F mutation. This suggested that the pathways of Aβ generation, especially with regards to Aβ42, are slightly different among the wild type, SW, and V717F forms of APP. Significantly, the mechanism for increased Aβ42 with APP codon 717 mutation may be different than that associated with the presenilin mutations. The results are submitted shortly (De Strooper B and Annaert W. J. Cell Sci. 113:1857-1870, 2000).

### EXAMPLE 14

Aβ is Reduced and APPs is Increased in Cells Lacking LRP in an Isoform Independent Manner

RATIONALE: The recent report that inhibiting or eliminating LRP resulted in marked reduction in Aβ secretion was unanticipated and exciting [Ulery P G, et al. J. Biol. Chem. 275:7410-7415, 2000]. At the same time, as mentioned previously, the KPI dependency of this model was surprising to us. Therefore, Inventors sought to confirm the findings reported and study the APP isoform relationship.

RESULTS: APP695 and APP751 were expressed in LRP deficient cells (LRP−/−) and corresponding LRP control cells (LRP±) by retroviral mediated infection. Single clones of comparable expression level were chosen for analyses of APPs and Aβ secretion. When the cell lysates were normalized to both protein and APP levels, the amount of APPs in LRP−/− cells was three-fold higher than in LRP± cells (FIG. 11a). Aβ in culture medium of LRPγ and LRP−/− cells transfected with APP751 was measured by ELISA using an A□40 end-specific monoclonal antibody. At both the 48 hr and 72 hr time intervals, LRP−/− cells secreted approximately 50-80% less Aβ than control LRP± cells (FIG. 11). These results in APP751 expressing LRP−/− cells are virtually identical to the Ulery report. Inventors next examined the same parameters in LRP−/− cells expressing APP695. Surprisingly, the particular APP isoform had no effect, i.e., essentially the same changes in APPs and Aβ levels were seen in cells transfected with APP695. Importantly, both Aβ40 and Aβ42 are affected to comparable degrees (not shown). Shown in this representative figure are ELISA measurements of Aβ levels in culture medium (FIG. 11a).

SIGNIFICANCE: In this study, Inventors confirmed the seminal findings that in the absence of LRP, secretion of APPs is significantly increased while Aβ (both Aβ40 and Aβ42) release is markedly reduced. Surprisingly, Inventors found that this effect is independent of the APP isoform. This suggested that processing of APP is not regulated exclusively through interaction of its KPI-domain with LRP because APP695 isoform lacking this domain also demonstrated LRP-dependent regulation of APPs and Aβ secretion.

### EXAMPLE 15

C. 4.2 Inhibition of APP CTFs in Cells Lacking LRP

RATIONALE: In view of the APPs and Aβ results from C.4. 1, Inventors next asked whether other aspects of APP processing are perturbed when LRP is absent. Because Aβ production, APPs secretion, and the generation of APP CTFs are frequently correlated, Inventors assayed for APP CTF in LRP± and LRP−/− cells stably transfected with either APP695 or APP751.

RESULTS: Analyzing clones of APP751 and APP695 transfected in LRP−/− and control LRP± cells with comparable expression, the levels of the a-secretase-cleaved APP CTF (C83) were consistently and significantly decreased in LRP−/− cells as compared to controls (FIG. 12a). Again, this alteration in the levels of APP CTF occurred in LRP−/− cells independent of the APP isoform (FIG. 12b). APP CTF levels were reduced an average of five-fold from multiple experiments in LRP−/− cells. The APP isoform independence indicated to Inventors that the KPI domain is not responsible for this marked difference in CTF generation. Because it has been proposed that the cytoplasmic domains of LRP and APP may interact with each other, Inventors engineered a truncated LRP construct beginning with the signal peptide together with the C-terminal 370 amino acids of LRP consisting of the transmembrane and cytoplasmic domains of LRP (LRP-CT). By surface biotinylation, this construct reaches the cell surface similarly to full length LRP (data not shown). Significantly, when this construct, LRP-CT, was expressed in LRP deficient cells, APP CTF and APPs were restored to control levels (FIG. 12b).

SIGNIFICANCE: The above results represent critical preliminary observations because they showed other steps in APP processing, in this instance, the level of APP CTFs, are LRP dependent. In addition, the results indicated that the cytoplasmic domain of LRP, at least the region encoded by the LRP-CT fragment, modulates APP processing.

### EXAMPLE 16

C.4.3 Cell Surface Internalization of APP is Impaired in Absence of LRP

RATIONALE: If the reduction in Aβ in LRP−/− cells is secondary to impaired endocytic processing, Inventors would expect to see changes in APP internalization in LRP−/− cells stably transfected with either APP695 or APP751. The prediction was that loss of APP internalization should parallel the reduction in Aβ release seen in the LRP−/− cells.

RESULTS: An APP internalization assay was performed in LRP-deficient cells expressing either APP695 or APP751 using a protocol established in Inventors' laboratory in which iodinated APP monoclonal antibody (1G7) was added to cultured cells at 37° C. for 30 minutes [Koo E H and Squazzo S L. J. Biol. Chem. 269:17386-17389, 1994]. Internalized and cell surface APP can be rapidly determined by the acid resistant and acid labile pools, respectively. In the LRP± cells transfected with APP695 or 751, APP was rapidly internalized from the cell surface, as has been shown for other cell types (data not shown). In contrast, after 30 minutes, the internalized pool of APP in LRP−/− cells was reduced by approximately 50% as compared to LRP ± cells (FIG. 13). This finding was present in both APP695 and APP751 expressing LRP−/− cells, again confirming that the changes are APP isoform independent (not shown). Inventors next asked if the reduction in APP internalization could be restored by expressing the LRP-CT construct. Consistent with the APP CTF results above (section C.4.2), the impairment in APP internalization can be restored to almost control levels, again confirming that the LRP-CT domain is necessary and sufficient to regulate this aspect of APP processing (FIG. 13). Finally, to correlate the changes with Aβ generation and release, LRP-CT expression in LRP−/− cells also restored the Aβ levels back to control conditions, in parallel with the restoration of APP cell surface internalization (not shown).

SIGNIFICANCE: Results from this study demonstrate that APP internalization is impaired in the absence of LRP, in parallel with the reduction in Aβ secretion first described by Ulery and colleagues and from Inventors' own experiments. Importantly, these perturbations in APP internalization and A□ production can be restored with LRP-CT, providing further evidence that the C-terminus of LRP is critical for regulating APP processing.

### EXAMPLE 17

C.4.4 LRP Affects the Turnover of APP and APP CTF Independently

RATIONALE: The marked reduction in the amount of APP-CTF in LRP deficient cells was unexpected. Inventors therefore determined the turnover of full-length APP and also that of APP CTFs to see if stability of the proteins are affected by loss of LRP.

RESULTS: The turnover of full length APP was determined by pulse chase analysis in LRP± and LRP−/− fibroblasts stably transfected with APP751 to determine if instability of APP can account for loss of CTFs. Unexpectedly, the turnover rate of APP in LRP−/− fibroblasts was slowed rather than increased as compared to control fibroblasts, indicating that LRP normally facilitates the rapid degradation of APP. The normal short half-life of APP (70 min) was almost doubled (120 min) in LRP deficient cells (FIG. 14). The reduced turnover of full length APP in LRP deficient cells is counterintuitive because increased stability of APP should have resulted in more, not less, APP-CTF. Therefore, the turnover of APP-CTF itself was analyzed. The cells were labeled for 1 hour, a duration that was found to result in comparable levels of CTFs in both LRP−/− and LRP± cells, indicating that generation of CTF was not impaired. However, APP-CTFs in LRP−/− were less stable. The turnover rate of APP-CTFs in LRP−/− cells was approximately twice that seen in LRP± cells, indicating that the low levels of APP-CTF in LRP−/− cells are due in part from reduced stability of the fragments in the absence of LRP (FIG. 14).

SIGNIFICANCE: The above results indicated that LRP normally facilitates the turnover of multiple APP species: both full length and CTFs. Inventors hypothesize that these occur in different compartments, an issue that are addressed in section D.1.2.

### EXAMPLE 18

C.4.5 The LRP C-terminus modulates APP processing

RATIONALE: To summarize, Inventors have showed that each LRP-dependent step in APP processing perturbed in the absence of LRP can be restored by the expression of the C-terminus of LRP (LRP-CT), and where tested, both APP695 and APP751 isoforms showed the same defects. These results provided strong evidence that the LRP C-terminal fragment, and potentially only the cytoplasmic domain, is important in the regulation of multiple steps in APP processing. A number of potentially important sites have been identified in the cytoplasmic tail of LRP, among which are two NPXY motifs that have been shown to interact with a family of cytoplasmic adaptor proteins [Howell B W, et al. Mol. Cell Biol. 19:5179-5188, 1999, Trommsdorff M, et al. J. Biol. Chem. 273:33556-33560, 1999]. However, the N-terminally truncated LRP-CT construct still contains about 230aa in the extracellular region where interaction with APP can theoretically take place, even though it does not contain any known ligand-binding domain. Therefore, Inventors needed to determine whether specific sequences or domains within the LRP cytoplasmic tail are necessary and sufficient for these effects.

RESULTS: To clarify which region in the C-terminus of LRP is involved in the modulation of APP processing, two LRP-CT mutants, each deleting the respective NPXY motif, were engineered. These were chosen so that each of the NPXY motifs would be removed separately. The two constructs, LRP-CTΔ1 (deleting residues 24-40) and LRP-CTΔ2 (minus 42-63), deleting approximately 20 amino acids each, were stably transfected into LRP −/− cells expressing APP751 (FIG. 15). As before, loss of LRP is associated with a large decrease in the levels of APP-CTFs. Interestingly, introduction of LRP-CTΔ1, but not LRP-CTΔ2, into LRP −/− cells restored APP-CTFs to control levels (FIG. 16). Similarly, LRP-CTΔ1, but not LRP-CTΔ2, also restored the levels of APPs to that seen in LRP +/− control cells (not shown).

SIGNIFICANCE: The preceding results indicated that the responsible regulatory sequences are contained within LRP-CTΔ2, or between amino acids 4486-4507 (or residues 41-63 of the 100 amino acids in the LRP cytoplasmic tail), as deletion of this region rendered the LRP-CT incapable of restoring the alterations in APP processing back to normal. This region contains the FTNPVY motif, or the second of the two NPXY motifs in the cytoplasmic tail (FIG. 15). Thus, this experiment established that it is specifically the cytoplasmic domain of APP that is critical for modulating APP trafficking. In Aim 1 (D.1.1), Inventors propose to narrow down the sequence within the LRP C-terminus responsible for these effects by smaller deletion constructs and site specific mutagenesis.

### EXAMPLE 19

Summary of Preliminary Studies

Inventors preliminary experiments showed that LRP normally modulates multiple steps in APP processing and trafficking, including the turnover of both full length and C-terminal fragments of APP, secretion of APPs, internalization of cell surface APP, and the generation and release of Aβ peptide. Significantly, many of the LRP dependent steps are independent of the KPI isoform of APP, as the processing of both APP695 and APP751 are abnormal in the absence of LRP. In view of the finding that deletion of the second NPXY motif in the cytoplasmic tail of LRP is essential for these effects, Inventors propose that the functional interaction between LRP and APP that influences APP processing occurs via the cytoplasmic rather than extracellular domains and is likely to take place in multiple cellular compartments (FIG. 1). To Inventors knowledge, this is the most extensive range of changes in APP processing that has been shown to be dependent on another molecule. The basis of the LRP dependent processing steps and whether this interaction can be exploited as a therapeutic avenue is explored herein below.

Rationale: Using deletions of approximately 20 amino acids, Inventors preliminary studies showed that certain sequences within the cytoplasmic domain of LRP are necessary and sufficient to regulate major aspects of APP trafficking and processing. Within each of the deleted regions are not only the NPXY motifs but also other potentially important sequences, such as tyrosine residues (FIG. 15) [Barnes H, et al. J. Biol. Chem. 276:19119-19125, 2001.]. Thus, to understand how LRP actually modulates APP processing, Inventors have to pinpoint the precise residue(s) that are critical for this LRP fuinction. In this subaim, Inventors identify these particular residues through a systematic deletion of smaller amino acid segments, followed by single site mutagenesis.

Experimental Design: The approach in this section is similar to that taken both in the past [Perez R G, et a. J. Biol. Chem. 274:18851-18856, 1999] and in the preliminary studies. As Inventors have initially narrowed the region to that deleted within LRP-CTΔ2, Inventors begin by performing smaller deletions within this 22 amino acid domain. Closer inspection of this sequence reveal that the second of the two NPXY motifs is right at the C-terminal end (-NPVY-). Interestingly, it was recently reported that the dominant signal for endocytosis of LRP is not the consensus NPXY motifs but the YATL motif [Li Y, et al. J. Biol. Chem. 275:17187-17194, 2000], beginning at position 66 (position 1 is the first residue of the 100 amino acid long cytoplasmic tail of LRP). This tyrosine residue overlaps with the second NPVY motif and is deleted in the LRP-CTΔ2 construct (FIG. 14). Accordingly, Inventors conduct three sets of experiments. First, Inventors delete the second NPVY (positions 60-63) and then the YATL (positions 63-66). The LRP-CT deletion constructs are then expressed in LRP −/− cells and APP processing (levels of CTF, APPs secretion, APP internalization) are assessed as before. The constructs are expressed in the CHO—K1 LRP −/− cells as before (clone 13-5-1, kindly given to Inventors by Dr. S. Leppla) [25]. Inventors focus on these particular sequences first because they rationalize that the NPVY motif should be the critical region, which is how Inventors designed the two large CTΔ1 and CTΔ2 deletions initially. However, if both deletions are able to restore the alterations in APP processing in LRP −/− cells, then it suggests that the critical sequence is outside of this 7 amino acid domain. In this case, Inventors start at the N-terminus of the original 22 amino acid region deleted in LRP-CTΔ2 by doing nested deletions to narrow down the critical residues. If one but not both tetrapeptide deletions cannot restore the defects in APP processing in LRP −/− cells, then Inventors proceed to alanine scanning mutagenesis through the appropriate tetrapeptide sequences. If both tetrapeptide deletions fail to restore APP processing, then it would suggest that the overlapping tyrosine (Holcomb L, et al. Nature Medicine. 4:97-100, 1998) is the critical residue. In this case, Inventors perform the following alanine mutagenesis: N60A, Y63A, and L66A. These three amino acids are chosen in order to distinguish between endocytosis of LRP (YATL) versus disruption of LRP:protein interactions, such as Fe65 which binds NPTY motifs [Guenette S Y, et al. Proc. Natl. Acad. Sci U.S.A. 93:10832-10837, 1996, Russo T, et al. FEBS Lett. 434:1-7, 1998]. In the third series of experiments, Inventors take the mutants engineered and tested in the above two experiments and perform co-immunoprecipitations (co-IP) with APP. Inventors and others have postulated that the interaction between APP and LRP is functionally important [Trommsdorff M, et al. J. Biol. Chem. 273:33556-33560, 1999, Ulery P G, et al. J. Biol. Chem. 275:7410-7415, 2000]. As reported, LRP complexes with APP [Rebeck G W, et al. Brain Res. Mol. Brain Res. 87:238-245, 2001]. Interestingly, Inventors can not only co-IP APP with LRP, but also with LRP-CT (FIG. 16). Therefore, each of the mutant LRP constructs expressed in LRP −/− cells are co-immunoprecipitated for APP. Inventors utilize polyclonal and monoclonal antibodies that recognize APP and LRP. Appropriate positive and negative controls are included, as Inventors have done in the past for presenilin with APP or β-catenin and presenilin complexes [Kang D E, et al. J. Neurosci. 19:4229-4237, 1999, Xia W, et al. Proc. Natl. Acad. Sci. USA. 94:8208-8213, 1997].

### EXAMPLE 20

Does LRP Regulate APP Processing Through its Cytoplasmic Domain Because of the Endocytic Signal

Rationale: As was pointed out by the Brown and Goldstein laboratories in their seminal study on the sequence required for rapid endocytosis of the LDLR, many cell surface receptors share the same NPXY motif in the cytoplasmic region, including APP, LRP, and GP330 (megalin, another LDLR family member closest to LRP) [Chen W -J, et al. J. Biol. Chem. 265:3116-3123, 1990]. Mutagenesis of either the N, P, Y, or the aromatic residue F, two residues N-terminal to the N residue, all reduced internalization by more than 60%. As described above (FIG. 14), LRP contains two such NPXY motifs: IGNPTY (aa 24-29 by numbering of the 100 aa cytoplasmic tail) and FTNPVY (aa 58-63). Thus, either could represent the internalization signal. Li and colleagues recently pointed out that in addition to the NPXY motifs, there are two other canonical internalization motifs in the LRP tail: two di-leucine (aa 43-44 and 86-87) and one YXXL (aa 63-66) motif [Li Y, et al. J. Biol. Chem. 275:17187-17194, 2000]. Their results using mini-LRP receptor constructs argue that the dominant signal for endocytosis is YXXL, with some contribution from the distal LL motif Thus, sequence analysis is only partially predictive, just as the dominant signal in APP is YENP rather than NPTY. Importantly, the YXXL motif overlaps with the FTNPVY motif, sharing a potentially critical tyrosine residue, which was deleted in Inventors LRP-CT□2 construct (C.4.5). With this background, Inventors test whether the endocytic signal within LRP is necessary and sufficient for its effect on APP by substituting the LDLR tail for LRP. If, on the other hand, the mechanism by which LRP affects APP processing is due to protein interactions independent of the internalization signal and unique to LRP, then the LRP-LDLR chimera is not able to restore APP processing altered in the absence of LRP. As a complementary experiment, Inventors express an APP-LDLR chimera in the same LRP −/− cells in order to ask whether the dependency on LRP can be bypassed if a different cytoplasmic tail is added to APP. It has been shown that an APP-LDLR chimera (exchanging the LDLR cytoplasmic tail for APP), is able to traffic quite normally, in fact, Aβ40 is increased two fold relative to wild type APP [Annaert W G, et al. J. Cell Biol. 147:277-294,1999]. In this way, one can define which domain within APP, rather than LRP, is required for the regulatory actions of LRP by doing the APP-LDLR cytoplasmic domain exchange.

Experimental Design: In this part, Inventors generate the LRP-LDLR chimera by substituting the 50 aa cytoplasmic tail from LDLR with the 100 aa cytoplasmic tail of LRP. The chimera are generated by PCR technique as Inventors have done before. The starting LRP construct is the same LRP-CT truncated construct that is effective in restoring alterations in APP processing when expressed in LRP −/− cells. Inventors designate the construct LRP-CT-LDLR. After the construct is engineered, it is expressed in the same LRP −/− cells where Inventors can assess processing of endogenous APP. However, to measure Aβ, Inventors need to overexpress APP in the LRP −/− cells as the levels are too low for consistent measurements. As a corollary, Inventors also generate the APP-LDLR chimera where the 47 amino acid cytoplasmic tail of APP is replaced by the 50 amino acid cytoplasmic tail of LDLR, exactly as was reported [Annaert W G, et al. J. Cell Biol. 147:277-294,1999]. This APP-LDLR receptor are expressed in LRP −/−, LRP −/− expressing LRP-CT, and control LRP +/+ cells. In LRP −/−, Inventors test whether the presence of the LDLR cytoplasmic tail is capable by itself to correct the defects in APP processing in the absence of LRP. In LRP-CT expressing cells, Inventors determine if the LDLR cytoplasmic tail is functionally exchangeable with the APP tail. Throughout this process, Inventors examine multiple parameters in APP processing, including APPs secretion and Aβ levels, APP internalization, and APP CTF levels as in section D.1.1. Since Aβ40 and Aβ42 appear to be coordinately affected in LRP, Inventors concentrate on measuring total Aβ for this and all subsequent experiments, unless otherwise indicated. As regards to APP CTF's, the C-terminal fragment from the APP-LDLR obviously be different from APP. Inventors accordingly use a LDLR C-terminal monoclonal antibody available from ATCC.

### EXAMPLE 21

LRP Affects APP Processing in Multiple Sites

Rationale: One approach to determine the mechanism by which LRP regulates APP processing is to examine the subcellular compartments where this interaction occurs. In Inventors preliminary studies, they confirmed that LRP can be co-immunoprecipitated with APP. Interestingly, Inventors saw that APP complexes with both full length LRP and the LRP β-subunit (FIG. 17). Because LRP undergoes furin cleavage in the TGN to generate the large α- and the smaller β-subunits [Herz J, et al. EMBO J. 9:1769-1776, 1990], Inventors results suggest that APP complexes with LRP in the early secretory compartments. If so, Inventors would predict that LRP influence APP in the ER/Golgi compartments. As these same compartments are major sites for the generation of APP CTFs [Annaert W G, et al. J. Cell Biol. 147:277-294,1999, Yan R, et al. J. Biol. Chem. 276:36788-36796, 2001], this could explain the observed changes in APP CTF levels in the absence of LRP. Because changes in APP internalization are likely to occur at the cell surface, Inventors hypothesize that LRP influences APP in multiple compartments.

Experimental Design: Inventors examine two different LRP mutants that mislocalize LRP and determine their effects on APP processing and APP:LRP complex formation. The hypothesis is that by complexing with APP, LRP influences APP processing in multiple compartments, not all of which are related to Aβ generation. In the first experiment, Inventors add an ER retention signal (KK at positions-3 and -4) at the C-terminus of LRP, much has been done successfully with APP and BACE [Cook D G, et al. Nature Medicine. 3:1021-1023, 1997, Huse J T, et al. J. Biol. Chem. 2002, Maltese W A, et al. J. Biol. Chem. 276:20267-20279, 2001]. As a result, this LRP mutant is localized predominantly in the ER when transfected into LRP −/− cells overexpressing wild type APP. The mislocalization is confirmed by immunofluorescence (with appropriate control markers such as BiP or calnexin) or biochemically (where LRP exist predominantly as full length species as well as by the failure to acquire endoglycosidase H resistance). In the second experiment, a mutant CHO cell line generated by and already obtained from Dr. S. Leppla (14-2-1) is examined. Instead of being deficient in LRP, the endogenous LRP in 14-2-1 cell line is retained primarily in the Golgi without being sorted to the cell surface [FitzGerald D J, et al. J. Cell Biol. 129:1533-1541, 1995]. Thus, Inventors can ask how retention of LRP in the intermediate compartments alters APP processing. Inventors predict that LRP dependent steps in APP processing beyond the Golgi compartment remain perturbed in the 14-2-1 cells. As before, Inventors examine multiple steps in is APP processing, including APPs secretion, Aβ levels in medium, turnover of full length APP, APP internalization, levels of APP CTF, and turnover of APP CTFs. Finally, Inventors correlate these changes to APP:LRP complex formation to determine if physical association between APP and LRP is a requirement.

### EXAMPLE 22

Characterize the Pathway or Compartment Where LRP Acts to Influence Aβ Generation.

Rationale: In this area, Inventors turn their attention to how LRP affects APP within the known pathways of Aβ generation. The reported evidence supports the view that several cellular pathways are responsible for the generation of Aβ: 1) Aβ, especially Aβ42, can be formed in the early secretory pathway but Aβ formed in the ER may be largely retained in the cell [Cook D G, et al. Nature Medicine. 3:1021-1023, 1997, Skovronsky D M, et al. ______ 141:1031-1039, 1999, Tienari P J, et al. Proc. Natl. Acad. Sci U.S.A. 94:41254130,1997, Wild-Bode C, et al. J. Biol. Chem. 272:16085-16088, 1997], 2) Aβ can be formed in the Golgi/TGN compartments and utilization of this pathway is enhanced in the case of the APP “Swedish” 670/671 mutation (APP-SW) [Citron M, et al. Nature. 360:672-674,1992, Haass C, et al. Nature Medicine. 1:1291-1296, 1995, Perez R G, et al. J. Biol. Chem. 271:9100-9107,1996, Thinakaran G, et al. J. Biol. Chem. 271:9390-9397, 1996], 3) Aβ can be formed from APP processed in the endocytic pathway following internalization from the cell surface, possibly in the endosomal compartments and TGN [Cescato R, et al. J. Neurochem. 74:1131-1139,2000, Daugherty B L and Green S A. Traffic. 2:908-916,2001, De Strooper B and Annaert W. J. Cell Sci. 113:1857-1870,2000, Koo E H and Squazzo S L. J. Biol. Chem. 269:17386-17389, 1994, LeBlanc A C and Gambetti P. Biochem. Biophys. Res. Commun. 204:1371-1380,1994]; and 4) Aβ can be generated from cholesterol rich microdomains (lipid rafts or detergent insoluble glycolipid-enriched fraction [DIG]) [Lee S -J, et al. Nature Medicine. 4:730-734, 1999, Morishima-Kawashima M and Ihara Y. Biochemistry. 37:15247-15253, 11-3-1998, Parkin E T,et al. J. Neurochem. 1534-1543, 1999, Riddell D R, et al. Current Biology. 11:1288-1293, 2001]. In this subaim, Inventors attempt to fit the role of LRP in Aβ genesis within the context of known pathways of Aβ production. Specifically, Inventors preliminary results led Inventors to hypothesize that reduction in Aβ levels in the absence of LRP is secondary to impaired APP internalization, and consequently, diminished endocytic processing. This hypothesis are tested in this aim.

Experimental Design: Up to this point, Inventors have concentrated on manipulating LRP to define the mechanism by which it affects APP processing. Here, Inventors concentrate on known APP mutants and determine how their processing is affected in the presence or absence of LRP. Specifically, since Inventors hypothesize that loss of LRP results in less APP processing in the endocytic pathway, Inventors predict that APP mutants that bypass the internalization pathway should therefore be unaffected by LRP deficiency. In the first set of experiments, Inventors express the APP-SW and the APP C-terminal fragment “C99” encoding the last 99 amino acids of APP which is generated after β-secretase cleavage. The Swedish mutation produces more Aβ but also renders the endocytic pathway less important in Aβ generation, most likely because the mutation is a better substrate for β-secretase cleavage such that APP is efficiently cleaved prior to exiting the Golgi [Citron M, et al. Nature. 360:672-674, 1992, Vassar R, et al. Science. 286:735-741, 1999]. C99 is essentially a β-secretase cleaved APP proteolytic fragment and it is therefore comparable to the Swedish mutation [Maltese W A, et al. J. Biol. Chem. 276:20267-20279, 2001]. Thus, deletion of the C-terminus in both APP-SW and C99, unlike wild type APP, reduces Aβ production by only a minor degree [Iwata H, et al. J. Biol. Chem. 276:21678-21685, 2001]. Inventors have confirmed the latter result using a C99 construct with two alanine substitutions in the YENP internalization motif of APP, i.e. (Y/A)EN(P/A). Whereas these alanine substitutions in full length APP create an internalization deficient mutant with substantially diminished Aβ secretion [Iwata H, et al. J. Biol. Chem. 276:21678-21685, 2001], the same mutations within C99 have very little effect on Aβ secretion (FIG. 18). Inventors express APP-SW and C99 in LRP −/− cells versus control LRP +/+ cells and determine if Aβ secretion is altered in the absence of LRP. The prediction is that if LRP does regulate APP internalization and is the mechanism by which Aβ levels are reduced in APP −/− cells, then loss of LRP should have only a minor effect on Aβ secretion from APP-SW and C99. Inventors also survey other steps in APP processing (turnover, CTFs, etc.) to see if these parameters are perturbed as well.

In the second experiment, Inventors examine the generation of Aβ in cholesterol rich microdomains. A number of laboratories have shown that lipid rafts are likely to represent intracellular sites of Aβ production. Inventors predict that if LRP influences APP trafficking to these sites as a way to modulate Aβ levels, then changes in APP localization, APP CTFs and conceivably even BACE and presenilins are altered in lipid rafts in the absence of LRP. Accordingly, Inventors isolate the buoyant rafts according to published methods and examine the levels of APP, APP CTFs (α- and β-secretase cleaved fragments), PS-1, BACE, and Aβ within these fractions [Lee S -J, et al. Nature Medicine. 4:730-734, 1999, Rockenstein E, et al. J. Neurosci. Res. 66:573-582, 2001]. Coincidently, one of the earliest reports of Aβ in lipid rafts used APP751 transfected CHO cells developed in Inventors laboratory [Lee S -J, et al. Nature Medicine. 4:730-734, 1999]. Thus, using a similar protocol, Inventors also documented the presence of full length APP and APP CTFs (α- and β-cleaved) in the low density fractions (FIG. 19). In addition to immunoblotting for the various APP related proteins (BACE, PS-1, APP CTFs), Inventors also measure Aβ levels by ELISA. Appropriate controls include raft markers such as flotillin and caveolin-1, but ER (calnexin) and Golgi (TGN38) markers are excluded.

### EXAMPLE 23

LRP Regulates APP Processing Via the Formation of an Adaptor Protein:APP:LRP Complex.

Rationale: In this set of experiments, Inventors initiate studies that provide, in molecular detail, the mechanism by which LRP regulates APP processing. At present, there is simply insufficient data to offer a parsimonious view as to how LRP may affect APP metabolism through the cytoplasmic domains. In fact, even a succinct model of Aβ generation from APP that incorporates the known APP interacting proteins is at present a difficult task. Nonetheless, Inventors can conceive of three working models by which LRP regulates APP processing. These all begin with the observation that the YENPTY motif in APP that is important for internalization and A□ secretion is the same site (with minor variations) for binding to a number of cytosolic proteins, including Fe65/Fe65L, X11 (or Mint-1)/X11L, JIP-1B (JNK-interacting protein), and mammalian disabled (mDab1) [Borg J P, et al. Mol. Cell Biol. 16:6229-6241, 1996, Fiore F, et al. J. Biol. Chem. 270:30853-30856, 1995, Homayouni R, et al. J. Neurosci. 19:7507-7515, 1999, Howell B W, et al. Mol. Cell Biol. 19:5179-5188,1999, Matsuda S, et al. J. Neurosci. 21:6597-6607, 2001, Russo T, et al. FEBS Lett. 434:1-7, 1998, Scheinfeld M H, et al. J. Biol. Chem. 277:3767-3775,2002, Van Gassen G, et al. Neurobiol. Dis. 7:135-151,2000].

Interestingly, Fe65, JIP-1, and mDab1 have also been shown to interact with LRP [Gotthardt M, et al. J. Biol. Chem. 275:25616-25624, 2000, Howell B W, et al. Mol. Cell Biol. 19:5179-5188,1999, Trommsdorff M, et al. J. Biol. Chem. 273:33556-33560, 1999]. Other APP interacting proteins such as PAT1, G_{O}, kinesin-1, and APP-BP1, either do not bind to this region or not enough is known about the actual binding site [Chow N W, et al. J. Biol. Chem. 271:11339-11346, 1996, Kamal A, et al. Neuron. 28:449-459, 2000, Nishimoto I, et al. Nature. 362:75-79, 1993, Zheng P, et al. Proc. Natl. Acad. Sci U.S.A. 95:14745-14750, 1998]. As it appears that the respective ψENPXY motifs (where ψ is any aromatic group) in both APP and LRP (this are elucidated in Aim D.1.1) are critical for Aβ generation, Inventors postulate the following three models: 1) APP and LRP must normally be complexed together via cytosolic adaptor molecules for normal APP processing; 2) APP and LRP are rapidly internalized via clathrin-coated vesicles, an interaction that is displaced by the cytoplasmic adaptor molecules that compete for binding to the same site; and 3) LRP regulates APP processing via an indirect mechanism that does not require complex formation between APP, LRP, or other cytosolic adaptor proteins. In the last scenario, the LRP cytoplasmic tail, putatively the NPXY motif, may participate in signal transduction pathway (such as tyrosine phosphorylation) that in turn alter APP processing [Barnes H, et al. J. Biol. Chem. 276:19119-19125, 2001]. Each of the three models have inherent strengths and weaknesses. Inventors have chosen here to focus on the first model. Inventors postulate that APP:cytosolic adaptor protein:LRP complexes are present in which APP and LRP do not interact directly (FIG. 1). Inventors further postulate that Fe65 or the related Fe65L is the key adaptor molecule because it is the only one reported to date with separate binding domains for both APP and LRP, creating a natural linker between the two proteins [Russo T, et al. FEBS Lett. 434:1-7, 1998, Trommsdorff M, et al. J. Biol. Chem. 273:33556-33560, 1999]. Fe65 and Fe65L possess two phosphotyrosine interacting domains (PID) and a “WW” domain that may function in nuclear transcription. PID1, the more N-terminal domain, binds to LRP whereas PID2, the C-terminal domain, binds to APP. Incidently, the “PID” or the equivalent phosphotyrosine binding “PTB” term is a bit of a misnomer as tyrosine phosphorylation is not required for binding to APP or LRP [Howell B W, et al. Mol. Cell Biol. 19:5179-5188,1999]. The role Fe65 plays in APP and LRP metabolism is not clear [Van Gassen G, et al. Neurobiol. Dis. 7:135-151, 2000]. Inventors own preliminary studies in CHO cells have replicated some of the published results in which overexpression of Fe65 augmented APPs secretion and a reduction in Aβ release. In this way, the changes in APPs and Aβ are reminiscent of the LRP deficient cells, suggesting that overexpression of Fe65 creates a dominant negative phenotype. For Fe65 to do this, Inventors postulate that both LRP and Fe65 are normally present in limiting amounts such that overexpression of Fe65 result in relative LRP deficiency. In this model, overexpression of another adaptor molecule (such as mDab1) compete with Fe65 for binding to APP [Lau K F, et al. NeuroReport. 11:3607-3610,2000], creating a state partially resembling LRP deficiency (FIG. 1). Consistent with this view, overexpression of mDab1 has been shown to reduce LDLR internalization [Gotthardt M, et al. J. Biol. Chem. 275:25616-25624, 2000]. Thus Inventors envision a situation where mDab1 or other proteins compete for the same substrates thereby inhibiting the APP:LRP interaction, resulting in a reduction both APP and LRP internalization. Accordingly, Inventors test this model of APP:LRP interaction occurring via cytosolic adaptor molecule Fe65 as the mechanism by which LRP regulates APP processing.

Experimental Design: Inventors test three predictions that derive from Inventors working model that the LRP regulation of APP processing occurs via a complex involving cytosolic adaptor proteins: 1) APP interacts with LRP in a Fe65-dependent fashion, 2) overexpression of Fe65 results in a partial LRP null phenotype, and 3) other cytosolic interacting molecules inhibit the APP:Fe65:LRP interaction. In the first experiment, Inventors examine the APP:LRP interaction. APP, LRP, and Fe65 can be colocalized by FRET [Kinoshita A, et al. J. Neurosci. 21:8354-8361, 2001] and APP:LRP can be co-immunoprecipitated [Rebeck G W, et al. Brain Res. Mol. Brain Res. 87:238-245,2001]. Inventors have also detected the same APP:LRP co-IP from transfected cells (FIG. 17) but it has not been demonstrated whether APP interacts directly with LRP. None of the reported 2-hybrid screens using the cytoplasmic tail of APP have detected interaction with LRP, and vice versa. Therefore, Inventors determine that Fe65 but not mDab1 is sufficient and necessary for this interaction by pull down and co-IP assays. GST fusion protein expressing the APP and LRP cytoplasmic tails are constructed. HEK 293 cells are transiently transfected with APP, LRP, Fe65, or mDab1 in various combinations indicated below. If antibodies are not available, then the protein are tagged by HA(hemagglutinin) or FLAG epitopes. The essential prediction is that APP can precipitate LRP in a Fe65 dependent manner but this interaction is inhibited by mDab1. For example, in the case using GST-APP to pull down LRP, Inventors perform the following:

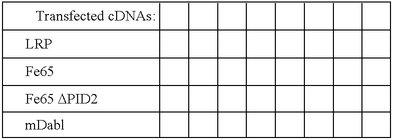

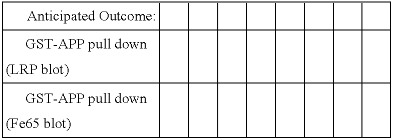

In this experiment, Inventors have added a mutant Fe65 ΔPID2 as a negative control as APP binding should be lost when the PID2 domain is deleted. The converse also is tested in which GST-LRP-tail is used as the affinity reagent and APP substitute for LRP in the transfection. Please note that the above schematic is a simplified scheme meant as an example only and other important negative controls were not included. But the general approach/strategy is apparent. The GST-pull down experiments subsequently are confirmed by co-IP from the transfected cells.

In the second set of experiments, Inventors determine whether overexpression of Fe65 impairs APP and LRP processing by acting as a dominant or competitive negative mutant. Inventors briefly mentioned the preliminary results above with APPs and Aβ when Fe65 was overexpressed in APP75 1-transfected CHO cells. These studies are repeated for reliable quantitation and other aspects of APP trafficking are examined, as performed in the earlier aims. Inventors also assess the binding and uptake of LRP ligands. To ascertain that these outcomes are due to inhibition by Fe65, Inventors overexpress Fe65 mutants that cannot bind either APP or LRP, i.e., ΔPID2 and ΔPID 1, respectively. If these mutant constructs mimic the results from full length Fe65 even though it cannot bind it's known substrate, then it supports the idea that Fe65 or Fe65L in excess behaves as an inhibitor. Finally, to demonstrate that LRP may be limiting, Inventors co-transfect Fe65 with full length LRP cDNA. The premise is that an overabundance of Fe65 result in the failure to link up limiting amounts of LRP with APP. Thus, co-expression of Fe65 with LRP should greatly diminish the effects of Fe65 expressed by itself In the third set of experiments, Inventors test the concept that other APP and LRP interacting proteins with only single binding sites, such as mDab, X11/Mint-1 and JIP-1, compete for binding with Fe65 or Fe65L, and by displacing Fe65, produce a partial loss of function with respect to APP processing. In the first experiment described above, Inventors have asked whether mDab1 overexpression reduce APP:LRP complex formation, presumably by displacing Fe65. In this section, Inventors extend those studies by asking whether overexpression of mDab1 alter APP processing, specifically APP internalization, APPs secretion, and Aβ release. If mDab1 does indeed function by competing for endogenous Fe65 substrate, then the effect are minimized by co-transfection with LRP.

### EXAMPLE 24

LRP can be Targeted to Inhibit Aβ Production.

Rationale: The preliminary results demonstrated that LRP has a major influence on APP processing that is likely to occur in multiple cellular compartments. To Inventor's knowledge, LRP's ability to regulate APP processing encompasses the most extensive changes that have been described to date of any proteins known to interact with APP. In this way, Inventors results significantly extended those reported by Ulery and colleagues and also demonstrated that the critical domain is in the LRP cytoplasmic tail. Whereas the mechanisms by which LRP modulates APP processing remain to be elucidated, and Aims 1 and 2 should provide significant insights into this question, Inventors results also argue that the APP:LRP interaction may represent a new approach whereby the production of Aβ can be targeted. In this regard, Inventors preliminary studies uncovered a paradoxic result. As described above, when LRP-CT is expressed in LRP −/− cells, there was partial to full restoration of the APP processing steps altered in the absence of LRP. In asking whether expression of LRP-CT in wild type cells (i.e. LRP +/+) results in an additive phenotype, as Inventors had anticipated, Inventors obtained the opposite result. Overexpression of LRP-CT in APP751-expressing CHO cells inhibited Aβ secretion, suggesting a dominant negative action resulting from high levels of LRP-CT. To confirm this result, Inventors isolated individual clones of LRP-CT stably expressed in APP751 cells and found an inverse correlation between LRP-CT expression and Aβ secretion (FIG. 11). Furthermore, transient expression of LRP-CT in human glioma and N2A mouse neuroblastoma cells likewise reduced Aβ secretion. Inventors therefore hypothesize that LRP-CT is only partially functional, or a partial loss-of-function mutant (not shown). In LRP −/− cells, expression at high levels produce a phenotype resembling normal cells but in wild type cells, overexpression results in a negative phenotype as it impairs the function of endogenous LRP. In this subaim, Inventors have two goals: test the concept that LRP-CT is a partial loss-of-function mutant and negatively influences LRP function and determine whether this finding can be exploited to inhibit Aβ levels in normal cells.

Experimental Design: In the first set of experiments, Inventors determine whether expression of LRP-CT, an artificial LRP C-terminus construct that is shorter than the normal LRP β-subunit, in wild type cells act to inhibit normal LRP function. Two approaches are taken. First, Inventors compare APP processing in APP751 CHO cells expressing either LRP-CT or only the 100 amino acid LRP cytoplasmic tail (LRP-tail) to that in LRP −/− cells. The CHO cells stably overexpressing LRP-CT have already been generated. CHO cells stably expressing LRP-tail are generated and both cell lines are characterized in detail. LRP-CT and LRP-tail act as a dominant or competitive negative inhibitor of endogenous LRP in CHO cells, therefore, APP processing, including Aβ secretion, is abnormal, much as Inventors have described in LRP −/− cells. In the second experiment, Inventors ask if uptake of LRP ligands are affected to assess the function of endogenous LRP. Over 20 ligands that bind to LRP have been described [Herz J and Strickland D K. J. Clin. Invest. 108:779-784, 2001], and for simplicity, Inventors assess two ligands: α-2-macroglobulin (α2M) and the 39 kDa receptor-associated protein (RAP). α2M is a normal physiologic ligand of LRP and RAP is an LRP chaperone molecule but has been used by many laboratories as an artificial LRP ligand [Li Y, et al. J. Biol. Chem. 275:17187-17194, 2000, Mikhailenko I, et a. J. Biol. Chem. 276:39484-39491,2001]. Inventors have previously generated a GST-RAP construct whereby RAP can be isolated after thrombin cleavage [Kang D E, et al. J. Clin. Invest. 106:1159-1166, 2000]. Both α2M (commercially available from Athens Research), activated by methylamine, and RAP are radiolabeled with ¹²⁵I and added to chilled cells at 4° C. according to established protocols. The cells are warmed to 37° C. and after 1, 3, and 5 hours, the medium and cells are harvested and specific surface binding, uptake, and degradation are measured. Inventors have extensive experience in monitoring the internalization of APP and Inventors use appropriate negative controls and competition experiments to ensure specific binding. A critical control is to express the LRP domains I and II mini-receptor because this truncated LRP construct is able to mediate high affinity binding and internalization of α2M [Mikhailenko I, et a. J. Biol. Chem. 276:39484-39491, 2001]. Thus, Inventors predict that expression of this construct (which Inventors have already generated), unlike LRP-CT, does not inhibit Aβ secretion. Lastly, Inventors perform co-immunoprecipitation experiments to determine if the expression of the “blocking” LRP sequence interferes with APP:LRP complex formation, as Inventors would predict.

In the second set of experiments, Inventors test whether smaller domains of the LRP cytoplasmic tail can be utilized to inhibit A□ production. The hypothesis is that if APP:LRP interaction can be perturbed, then APP processing and A□ generation are impaired. As Inventors have seen that expression of the entire cytoplasmic tail of LRP produces a “competitive negative” phenotype in various cell lines, this concept is tenable. Accordingly, Inventors deliver “blocking peptides” into cultured cells to inhibit APP processing and A□ production. The discovery and optimization of several protein transduction domains have facilitated the delivery of macromolecules not only in cultured cells but also in vivo. The three most popular are the Drosophila homeotic transcription protein antennapedia (Antp), herpes simplex VP22 structural protein, and the HIV-1 transcription activator Tat protein [101]. In collaborative studies with Dr. Dale Bredesen on the APP “C31” peptide which is released after caspase cleavage of APP (see section C.3.1), C31 fused to Antp (made as a synthetic peptide) can be efficiently transduced into both primary neurons and astrocytes. Recently, Inventors have turned to the Tat-based transducing fusion proteins [Schwarze S R, et al. Science. 285:1569-1572, 1999, Schwarze S R, et al. Trends Cell Biol. 10:290-295, 2000]. Unlike Antp peptides, this method obviates the need to synthesize long peptides, as Tat fusion proteins are easily produced in large quantities from bacterial cells and fusion proteins up to 100 kDa can be successfully delivered. With the Tat system, essentially 100% of cultured cells, including primary neurons, can be transduced, a property not possible with conventional transfection techniques. Inventors have the necessary vectors, and as seen from a Tat: □-galactosidase control, CHO and N2a neuroblastoma cells are readily transduced at almost 100% efficiency in the 100-200 nM concentration (FIG. 21). Initially, Inventors express the entire LRP cytoplasmic tail as a Tat fusion protein. Inventors start with the larger 68 amino acid Tat leader that includes that Tat sequence, a His tag for nickel column purification, and a HA epitope tag. Initially, Inventors place the tag at the N-terminus but may switch to the C-terminus if the fusion protein activity is sterically hindered. They follow the protocol established in the Dowdy lab to express and purify Tat fusion proteins in bacteria. Inventors first task is to determine if the Tat:LRP fusion protein mimic the results obtained by standard mammalian transfection technique. Then Inventors begin deletions from the N- and C-termini to narrow down to the minimal effective domain. Again, Inventors assess not only A□ secretion, but also APP processing as well as binding and internalization of LRP ligands (as performed above) to determine the degree to which LRP function has been inhibited. In addition, Inventors ascertain that the fusion proteins are indeed taken up by biochemical or immunofluorescence assays. Then Inventors proceed to test the Tat:LRP fusion peptides in other cell types, including primary neurons, to see if the results are more generalizable. Primary neurons are cultured from APP transgenic mice obtained from Dr. Eliezer Masliah (see section below) [Marquez-Sterling N R, et al. J. Neurosci. 17:140-151, 1997, Perez R G, et al. J. Neurosci. 17:9407-9414, 1997].

### EXAMPLE 25

Blocking APP:LRP Interaction is Effective in Inhibiting Aβ Production and Amyloid Pathology In Vivo.

Rationale: In the preceding experiments, Inventors tested the hypothesis that interference with the APP:LRP interaction can reduce the secretion of A□ from cultured cells. Here, Inventors conduct a proof-of-principle experiment in vivo. The concept that LRP, as a major regulator of APP processing and Aβ production, can be targeted for Aβ reduction needs to be tested in animals. LRP is a receptor for many ligands and genetic knockout of LRP leads to embryonic lethality [Herz J, et al. Cell. 71:411-421, 1992]. Thus, it is likely that inhibiting APP:LRP interaction negatively impacts LRP function.

Experimental Design: In previous experiments, Inventors characterized in detail the effects of LRP-CT and LRP-tail on APP processing, Aβ secretion, and uptake of LRP ligands in cultured cells. Here, Inventors express the LRP cytoplasmic tail in transgenic mice and determine if amyloid pathology in brain is altered. For the in vivo studies, Inventors express the smallest domain that is effective in reducing Aβ secretion on the premise that interfering with the entire LRP cytoplasmic tail is likely to result in greater inhibition with LRP function and hence more adverse cellular effects. To do this, the results using Tat:fusion protein are very helpful as they have narrowed the region that is effective in inhibiting Aβ secretion. Therefore, the first step is to express these small domains in cultured cells by transfection into various APP expressing cell lines (CHO, 293, and neuroblastoma). A tag, such as HA or myc, can also be added to differentiate the small peptides from endogenous LRP. Based on Inventors initial experience with LRP-CT, these studies replicate the Tat: LRP fusion protein results, and Inventors will proceed to in vivo experiments. For this, Inventors propose to generate transgenic mice that express the same domain (herein designated as “LRP-tail domain”) taken from the LRP cytoplasmic tail driven by a neuron-specific promoter. Inventors predict that the LRP-tail domain is a small fragment of the 100 amino acid LRP cytoplasmic tail. The hope is that the LRP-tail domain can be expressed at high levels, is not toxic, and allows Inventors to test the hypothesis that its presence blocks Aβ production and reduces the amyloid pathology in brains of APP transgenic mice. Dr. Eliezer Masliah at UCSD recently generated several interesting lines of APP transgenic mice, one of which (line 41) develops is plaque pathology as early as 3-4 months age [Rockenstein E, et al. J. Neurosci. Res. 66:573-582,2001]. These animals express human APP751 containing both the “Swedish” 670/671 KM-NL) and the “London” V7171) mutations driven by the mouse Thy-1 promoter. Other transgenic lines also develop early plaque pathology, but they either involve two crosses (e.g., APP×PS1 mutant) [Holcomb L, et al. Nature Medicine. 4:97-100, 1998], show unpredictable early lethality [Chishti M A, et al. J. Biol. Chem. 276:21562-21570, 2001], or Inventors do not have access to them. Inventors are already working with the Line 41 animals as part of another study.

Accordingly, Inventors express the LRP-tail domain in transgenic mice driven by the murine Thy-1 promoter. Protocols for generating transgenic mice are now well established. Inventors have had success generating various PS1 transgenic mice and tetracycline inducible APP and PS1 transgenic mice [Berezovska O, et al. Mol. Brain Res. 69:273-280, 1999, Kang D E, et al. J. Neurosci. 19:42294237, 1999, Xia X, et al. Proc. Natl. Acad. Sci U.S.A. 98:10863-10868, 2001](see below). Thus, this aspect is relatively straightforward. The LRP-tail domain construct (examine and tested in aim D.3.1 and above) is inserted into the mouse Thy-1 expression vector. Pronuclear injections (C57BL/6×DBA/2 F1) are carried out. The outcross strain of mice is the same used to generate the APP Line 41 mice. Founders are analyzed from tail bleeds by PCR and Southern blotting techniques. Founders are bred and positive animals from subsequent litters are analyzed for RNA (northern blotting) and protein (western blotting) expression. Lines that express varying levels of LRP-tail domain are expanded and then crossed to Line 41 APP transgenic mice. Inventors analyze only mice heterozygous for each transgene and compare non-transgenic littermates (APP expression alone and LRP-tail domain alone without APP). Because both the APP and LRP-tail domain transgenes are driven by the same promoter, Inventors are able to target similar neuronal populations. It should be noted that in brain, neurons represent the site of highest LRP expression. Inventors analyze mice between 10-12 months of age when amyloid pathology is quite extensive [Rockenstein E, et al. J. Neurosci. Res. 66:573-582, 2001]. Indeed, at this age, plaque burden, i.e., area covered by amyloid deposits, reaches 10 percent—an amount equal to or slightly higher than that seen in AD individuals. Power calculations from Inventors NSAID study showed that for even a 20% reduction in plaque number, five mice are sufficient for 80% confidence [Weggen S, et al. Nature. 414:212-216, 2001]. Inventors start with eight animals in each condition (experimental and littermate controls). The extra animals compensate for early death, etc. However, sufficient animals are housed so that older ages are available for analysis after the initial series have been examined. The weights of the animals are recorded and general health monitored in cooperation with the University veterinary staff. The brains of animals are divided in half sagitally: one hemisphere is snap frozen while the other is fixed in 4% paraformaldehyde, cryoprotected with sucrose and sectioned for morphological analyses. Inventors survey a number of biochemical and morphological parameters in brain. A□ levels are determined from the frozen hemispheres by ELISA. For the morphological measurements, Inventors are assisted by Dr. Eliezer Masliah, whose laboratory is very experienced in examining the brains of APP transgenic animals. Immunohistochemistry for amyloid plaques using A□ specific antibodies (both total A□ and end-specific A□40 and A□42 antibodies), anti-ubiquitin to detect dystrophic neurites, GFAP to visualize reactive astrocytes, and anti-synaptophysin are performed. Inventors estimate plaque densities, synaptophysin-positive synaptic terminals, and dystrophic neurites from representative brain sections. Inventors follow the general approach reported by the Masliah laboratory [Matsuda S, et al. J. Neurosci. 21:6597-6607, 2001].

In the above approach, there is one potential problem. Inventors are mindful that constitutive expression of LRP-tail domain in mice may be harmful or lethal to the animals. This is because the blocking peptide may effectively inhibit LRP function and genetic deficiency in LRP is embryonically lethal [Herz J, et al. Cell. 71:411-421, 1992]. Thus, Inventors take an alternative approach by expressing LRP-tail domain under tetracycline regulation. This method has been used successfully by many laboratories but for neuron specific expression, current technology is still largely restricted to the CamKIIα driven system pioneered by Dr. Mark Mayford [Mayford M, et al. 274:1678-1683, 1996, Yamamoto A, et al. Cell. 101:57-66, 2000]. Inventors collaborated with Dr. Mayford when he was at UCSD to generate several lines of tet-inducible animals under the CamKIIα promoter. In animals expressing human APP, the highest expression was three-fold over endogenous: not sufficient for plaque deposition but certainly quite high in absolute protein levels. In comparison, the YAC APP transgenic mice express at levels equal to endogenous APP [Lamb B T, et al. Nature Neuroscience 2:695-697, 1999]. Other lines Inventors have successfully generated include wild type and mutant PS1 (FIG. 22) and BACE. In Inventors hands, the CainKIIα promoter directs expression of the transgene not only to the hippocampus, but also rather diffusely in neurons of the cortex. Inventors generate LRP-tail domain animals driven by the minimal tetO promoter which by itself does not express any protein. When crossed to the CamKIIα-tTA animals, the resulting double transgenic mice express the LRP transgene in the absence of tetracycline. When doxycycline is given, then expression is inhibited [Mayford M, et al. Current Biology. 7:R580-R589, 1997]. Both lines are crossed to the APP transgenic mice (line 41), a slightly more cumbersome but unavoidable breeding scheme. The plan is to activate expression of the LRP-tail domain transgene in late adult life. This way, adverse effects due to developmental inhibition of LRP function are avoided and the triple transgenic mice may survive to late adult life to be analyzed as outlined above.

### SUMMARY

1. The preceding findings argue that a small region encompassing the distal NPTYATL motif (residues 4504 to 4510) is necessary for the regulation of APP processing by LRP. To date, there is no evidence that LRP interacts directly with APP even though APP and LRP can be co-immunoprecipitated together. It has been proposed that adaptor molecules such as Fe65 can link APP to LRP. This was specifically proposed by Trommsdorf and colleagues in 1999 and is an attractive model because Fe65 contains two binding regions, PID1 and PID2, that interact with LRP and APP, respectively. However, no data was provided in that study for this model. And critically, it was specifically postulated that Fe65 binds to the upstream NPVY motif at residues 4470 to 4473. If this is true, then Fe65 could not be the adaptor protein that links LRP with APP to modulate the processing steps we have observed because the upstream NPVY motif is not needed in our cellular assays. As a result, co-immunoprecipitations and GST pull down assays were performed with various LRP-CT constructs lacking either the first or the second NPXY region, respectively. Unexpectedly, our results showed that Fe65 interacted with both NPXY motifs as deletions through either but not both NPXY regions in LRP were able to co-immunoprecipitate Fe65 from transfected cells. Pull down assay with GST fusion proteins encoding the last 100 amino acids of LRP or the entire cytoplasmic tail of LRP containing single alanine substitutions at N, Y, and L confirmed this observation. Specifically, all the alanine mutations within the NPTY region reduced the binding to in vitro translated Fe65 by over 50% as compared to wild type LRP. Importantly, this corresponded precisely to the transfection studies wherein LRP-CT containing these same alanine substitutions were unable to restore the abnormalities in APP processing in LRP deficient cells. Taken together, these findings suggested that the second NPTY motif is indeed important for the interaction with Fe65, contradicting the model proposed by Trommsdorf and colleagues.

4. If this proposed model is correct, then another prediction is that displacement of Fe65 from LRP or Fe65 from APP can be the basis of a screening assay for compounds that lower Aβ generation. Accordingly, LRP (or APP) and Fe65 represent the necessary substrate and reporter pair that is required for a high throughput assay. If LRP (or APP) represents the substrate, then Fe65 can be tagged in a way to function as a sensitive reporter: loss of binding can be readily detected and represents loss of the LRP (or APP):Fe65 interaction. Consequently, compounds that displace the normal interaction between LRP (or APP) and Fe65 respectively, should in theory also inhibit this interaction in vivo. If so, this then represents the basis of a high throughput assay for screening compounds that inhibit Aβ production through this mechanism.

While the present invention has now been described in terms of certain preferred embodiments, and exemplified with respect thereto, one skilled in the art will readily appreciate that various modifications, changes, omissions and substitutions may be made without departing from the spirit thereof It is intended, therefore, that the present invention be limited solely by the scope of the following claims.

References

Ando K, Iijima K I, Elliott J I, Kirino Y, Suzuki T. Phosphorylation-dependent regulation of the interaction of amyloid precursor protein with Fe65 affects the production of β-amyloid. J. Biol. Chem. 276:40353-40361, 2001.

Annaert W G, Levesque L, Craessaerts K, Dierinck I, Snellings G, Westaway D, George-Hyslop P S, Cordell B, Fraser P, De Strooper B. Presenilin 1 controls γ-secretase processing of amyloid precursor protein in pre-golgi compartments of hippocampal neurons. J. Cell Biol. 147:277-294,1999.

Barnes H, Larsen B, Tyers M, van Der Geer P. Tyrosine-phosphorylated low density lipoprotein receptor-related protein 1 (Lrp1) associates with the adaptor protein SHC in SRC-transformed cells. J. Biol. Chem. 276:19119-19125,2001.

Berezovska O, Frosch M P, McLean P, Knowles R, Koo E, Kang D E, Shen J, Lu F M, Lux S E, Tonegawa S, Hyman B T. The Alzheimer-related gene presenilin 1 facilitates notch 1 in primary mammalian neurons. Mol. Brain Res. 69:273-280,1999.

Borg J, Yang Y, De Taddeo-Borg M, Margolis B, Turner R S. The X11 protein slows cellular amyloid precursor protein processing and reduces Ab40 and Ab42 secretion. J. Biol. Chem. 273,No24:14761-14766, 1998.

Borg J P, Ooi J, Levy E, Margolis B. The phosphotyrosine interaction domains of X11 and FE65 bind to distinct sites on the YENPTY motif of amyloid precursor protein. Mol. Cell Biol. 16:6229-6241, 1996.

Bu G and Rennke S. Receptor-associated protein is a folding chaperone for low density lipoprotein receptor-related protein. J. Biol. Chem. 271:22218-22224, 1996.

Buxbaum J D, Liu K, Luo Y, Slack J L, Stocking K L, Peschon J H, Johnson R S, Castner B J, Cerretti D P, Black R A. Evidence that tumor necrosis factor α converting enzyme is involved in regulated α-Secretase cleavage of the Alzheimer amyloid protein precursor. J. Biol. Chem. 273:27765-27767, 1998.

Cao, X., and T. C. Sudhof (2001) A transcriptively active complex of APP with Fe65 and histone acetyltransferase Tip60. Science., 293, 115-20.

Cescato R, Dumermuth E, Spiess M, Paganetti P A. Increased generation of alternatively cleaved β-amyloid peptides in cells expressing mutants of the amyloid precursor protein defective in endocytosis. J. Neurochem. 74:1131-1139, 2000.

Chen W -J, Goldstein J L, Brown M S. NPXY, a sequence often found in cytoplasmic tails, is required for coated-pit mediated internalization of the low density lipoprotein receptor. J. Biol. Chem. 265:3116-3123, 1990.

Chishti M A, Yang D S, Janus C, Phinney A L, Home P, Pearson J, Strome R, Zuker N, Loukides J, French J, Turner S, Lozza G, Grilli M, Kunicki S, Morissette C, Paquette J, Gervais F, Bergeron C, Fraser P E, Carlson G A, George-Hyslop P S, Westaway D. Early-onset amyloid deposition and cognitive deficits in transgenic mice expressing a double mutant form of amyloid precursor protein 695. J. Biol. Chem. 276:21562-21570, 2001.

Chow N W, Korenberg J R, Chen X N, Neve R L. APP-BP1, a novel protein that binds to the carboxyl-terminal region of the amyloid precursor protein. J. Biol. Chem. 271:11339-11346, 1996.

Citron M, Oltersdorf T, Haass C, McConologue K, Hung A Y, Seubert P, Vigo-Pelfrey C, Lieberburg I, Selkoe D J. Mutation of the β-amyloid precursor protein in familial Alzheimer's disease increases Aβ-protein production. Nature. 360:672-674, 1992.

Cook D G, Forman M A, Sung J C, Leight S, Kolson D L, Iwatsubo T, Lee V M Y, Doms R W. Alzheimer's A□(1-42) is generated in the endoplasmic reticulum/intermediate compartment of NT2N cells. Nature Medicine. 3:1021-1023, 1997.

Cupers P, Bentahir M, Craessaerts K, Orlans I, Vanderstichele H, Saftig P, De Strooper B, Annaert W. The discrepancy between presenilin subcellular localization and □-secretase processing of amyloid precursor protein. J. Cell Biol. 154:731-740, 2001.

Daugherty B L and Green S A. Endosomal sorting of amyloid precursor protein-P-selectin chimeras influences secretase processing. Traffic. 2:908-916,2001.

Davis C G, van Driel I R, Russell D W, Brown M S, Goldstein J L. The low density lipoprotein receptor: identification of amino acids in cytoplasmic domain required for rapid endocytosis. J. Biol. Chem. 262:4075-4082, 1987.

De Strooper B and Annaert W. Presenilins and the intramembrane proteolysis of proteins: facts and fiction. Nature Cell Biology. 3:E221-E225, 2001.

De Strooper B and Annaert W. Proteolytic processing and cell biological functions of the amyloid precursor protein. J. Cell Sci. 1 13:1857-1870, 2000.

De Strooper B, Annaert W, Cupers P, Saftig P, Craessaerts K, Mumm J S, Schroeter E H, Schrijvers V, Wolfe M S, Ray W J, Goate A, Kopan R. A presenillin-1-dependent γsecretase-like protease mediates release of Notch intracellular domain. Nature. 398:518-522, 1999.

Dingwall C. Spotlight on BACE: the secretases as targets for treatment in Alzheimer disease. J. Clin. Invest. 108:1243-1246, 2001.

Dingwall, C. and G. Christie. (1999) Identification of a novel aspartic protease (Asp 2) as β-secretase. Mol. Cell. Neurosci., 14, 419-27.

Esler W P, Kimberly W T, Ostaszewski B L, Diehl T S, Moore C L, Tsai J Y, Rahmati T, Xia W, Selkoe D J, Wolfe M S. Transition-state analogue inhibitors of γsecretase bind directly to presenilin-1. Nat. Cell Biol. 2:428-434,2000.

Fassbender K, Simons M, Bergmann C, Stroick M, Lutjohann D, Keller P, Runz H, Kuhl S, Bertsch T, von Bergmann K, Hennerici M, Beyreuther K, Hartmann T. Simvastatin strongly reduces levels of Alzheimer's disease β-amyloid peptides Aβ42 and Aβ40 in vitro and in vivo. Proc. Natl. Acad. Sci U.S.A. 98:5856-5861, 2001.

Fiore F, Zambrano N, Minopoli G, Donini V, Dulio A, Russo T. The regions of the Fe65 protein homologous to the phosphotyrosine interaction/phosphotyrosine binding domain of Shc bind the intracellular domain of the Alzheimer's amyloid precursor protein. J. Biol. Chem. 270:30853-30856, 1995.

FitzGerald D J, Fryling C M, Zdanovsky A, Saelinger C B, Kounnas M, Winkles J A, Strickland D, Leppla S. Pseudomonas exotoxin-mediated selection yields cells with altered expression of low-density lipoprotein receptor-related protein. J. Cell Biol. 129:1533-1541, 1995.

Gervais F G, Xu D, Robertson G S, Vaillancourt J P, Zhu Y, Huang J, LeBlanc A, Smith D W, Rigby M, Shearman M S, Clarke E E, Zheng H, Van der Ploeg H T, Ruffolo S C, Thomberry N A, Xanthoudakis S, Zamboni R J, Roy S, Nicholson D W. Involvement of Caspases in proteolytic cleavage of Alzheimer's amyloid-β precursor protein and amyloidogenic A□ peptide formation. Cell. 97:395-406,1999.

Gotthardt M, Trommsdorff M, Nevitt M F, Shelton J, Richardson J A, Stockinger W, Nimpf J, Herz J. Interactions of the low density lipoprotein receptor gene family with cytosolic adaptor and scaffold proteins suggest diverse biological functions in cellular communication and signal transduction. J. Biol. Chem. 275:25616-25624, 2000.

Guenette S Y, Chen J, Ferland A, Haass C, Capell A, Tanzi R E. hFE65L influences amyloid precursor protein maturation and secretion. J. Neurochem. 73:985-993, 1999.

Guenette S Y, Chen J, Jondro P D, Tanzi R E. Association of a novel human FE65-like protein with the cytoplasmic domain of the β-amyloid precursor protein. Proc. Natl. Acad. Sci U.S.A. 93:10832-10837, 1996.

Haass C, Koo E H, Mellon A, Hung A Y, Selkoe D J. Targeting of cell-surface β-amyloid precursor protein to lysosomes: alternative processing into amyloid-bearing fragments. Nature. 357:500-503, 1992.

Haass C, Lemere C A, Capell A, Citron M, Seubert P, Schenk D, Lannfelt L, Selkoe D J. The Swedish mutation causes early-onset Alzheimer's disease by β-secretase cleavage within the secretory pathway. Nature Medicine. 1:1291-1296, 1995.

Hartmann, T., S. C. Bieger, B. Bruhl, P. J. Tienari, N. Ida, D. Allsop, G. W. Roberts, C. L. Masters, C. G. Dotti, K. Unsicker, and K. Beyreuther. (1997) Distinct sites of intracellular production for Alzheimer's disease A beta40/42 amyloid peptides. Nat. Med., 3, 1016-20.

Herz J and Strickland D K. LRP: a multifunctional scavenger and signaling receptor. J. Clin. Invest. 108:779-784, 2001.

Herz J, Clouthier D, Hammer R E. LDL Receptor-Related Protein Internalizes and Degrades uPA-PAI-1 Complexes and is Essential for Embryo Implantation. Cell. 71:411-421, 1992.

Herz J, Hamann U, Rogne S, Myklebost O, Gausepohl H, Stanley K K. Surface location and high affinity for calcium of a 500-kd liver membrane protein closely related to the LDL-receptor suggest a physiological role as lipoprotein receptor. EMBO J. 7:4119-4127, 1988.

Herz J, Kowal R C, Goldstein J L, Brown M S. Proteolytic processing of the 600 kd low density lipoprotein receptor-related protein (LRP) occurs in a trans-golgi compartment. EMBO J. 9:1769-1776, 1990.

Hiesberger, T., M. Trommsdorff, B. W. Howell, A. Goffinet, M. C. Mumby, J. A. Cooper, and J. Herz. (1999) Direct binding of Reelin to VLDL receptor and ApoE receptor 2 induces tyrosine phosphorylation of disabled-1 and modulates tau phosphorylation. Neuron., 24, 481-9.

Holcomb L, Gordon M N, McGowan E, Yu X, Benkovic S, Jantzen P, Wright K, Saad I, Mueller R, Morgan D, Sanders S, Zehr C, O'Campo K, Hardy J, Prada C -M, Eckman C, Younkin S, Hsiao K, Duff K. Accelerated Alzheimer-type phenotype in transgenic mice carrying both mutant amyloid precursor protein and presenilin 1 transgenes. Nature Medicine. 4:97-100, 1998.

Holtzman, D. M., R. E. Pitas, J. Kilbridge, B. Nathan, R. W. Mahley, G. Bu, and A. L. Schwartz. (1995) Low density lipoprotein receptor-related protein mediates apolipoprotein E-dependent neurite outgrowth in a central nervous system-derived neuronal cell line. Proc. Natl. Acad. Sci. USA., 92, 9480-4.

Homayouni R, Rice D S, Sheldon M, Curran T. Disabled-1 binds to the cytoplasmic domain of amyloid precursor-like protein 1. J. Neurosci. 19:7507-7515, 1999.

Horn, I. R., B. M. van den Berg, P. Z. van der Meijden, H. Pannekoek, and A. J. van Zonneveld. (1997) Molecular analysis of ligand binding to the second cluster of complement-type repeats of the low density lipoprotein receptor-related protein. Evidence for an allosteric component in receptor-associated protein-mediated inhibition of ligand binding. J. Biol. Chem., 272, 13608-13.

Howell B W, Lanier L M, Frank R, Gertler F B, Cooper J A. The disabled 1 phosphotyrosine-binding domain binds to the internalization signals of transmembrane glycoproteins and to phospholipids. Mol. Cell Biol. 19:5179-5188, 1999.

Huse J T, Liu K, Pijak D S, Carlin D, Lee V M, Doms R W. β-secretase processing in the trans-Golgi network preferentially generates truncated amyloid species that accumulate in Alzheimer's disease brain. J. Biol. Chem. 2002.

Huse J T, Pijak D S, Leslie G J, Lee V M, Doms R W. Maturation and endosomal targeting of β-site amyloid precursor protein-cleaving enzyme. The Alzheimer's disease β-secretase. J. Biol. Chem. 275:33729-33737, 2000.

Hussain, I., D. Powell, D. R. Howlett, D. G. Tew, T. D. Meek, C. Chapman, I. S. Gloger, K. E. Murphy, C. D. Southan, D. M. Ryan, T. S. Smith, D. L. Simmons, F. S. Walsh,

Hyman B T, Strickland D, Rebeck G W. Role of the low-density lipoprotein receptor-related protein in β-amyloid metabolism and Alzheimer disease. Arch. Neurol. 57:646-650, 2000.

Ikezu T, Trapp B D, Song K S, Schlegel A, Lisanti M P, Okamoto T. Caveolae, plasma membrane microdomains for a-secretase-mediated processing of the amyloid precursor protein. J. Biol. Chem. 273:10485-10495, 1998.

Iwata H, Tomita T, Maruyama K, Iwatsubo T. Subcellular compartment and molecular subdomain of β-amyloid precursor protein relevant to the Aβ42-promoting effects of Alzheimer mutant presenilin 2. J. Biol. Chem. 276:21678-21685, 2001.

Kamal A, Stokin G B, Yang Z, Xia C H, Goldstein L S. Axonal transport of amyloid precursor protein is mediated by direct binding to the kinesin light chain subunit of kinesin-I. Neuron. 28:449-459, 2000.

Kang D E, Pietrzik C U, Baum L, Chevallier N, Merriam D E, Kounnas M Z, Wagner S L, Troncoso J C, Kawas C, Katzman R, Koo E H. Modulation of amyloid β-protein clearance and Alzheimer's disease susceptibility by the LDL receptor-related protein pathway. J. Clin. Invest. 106:1159-1166, 2000.

Kang D E, Soriano S, Frosch M P, Collins T, Naruse S, Sisodia S S, Leibowitz G, Levine F, Koo E H. Presenilin 1 facilitates the constitutive turnover of β-catenin: differential activity of Alzheimer's disease-linked PS1 mutants in the β-catenin signaling pathway. J. Neurosci. 19:4229-4237, 1999.

Katzman R. Alzheimer's disease. New Engl. J. Med. 314:964-973, 1986.

Kinoshita A, Whelan C M, Smith C J, Mikhailenko I, Rebeck G W, Strickland D K, Hyman B T. Demonstration by fluorescence resonance energy transfer of two sites of interaction between the low-density lipoprotein receptor-related protein and the amyloid precursor protein: role of the intracellular adapter protein Fe65. J. Neurosci. 21:8354-8361, 2001.

Knauer, M. F., R. A. Orlando, and C. G. Glabe. (1996) Cell surface APP751 forms complexes with protease nexin 2 ligands and is internalized via the low density lipoprotein receptor-related protein (LRP). Brain Research., 740, 6-14.

Kojro E, Gimpl G, Lammich S, Marz W, Fahrenholz F. Low cholesterol stimulates the nonamyloidogenic pathway by its effect on the α-secretase ADAM 10. Proc. Natl. Acad. Sci U.S.A. 98:5815-5820, 2001.

Koo E H and Squazzo S L. Evidence that production and release of amyloid β-protein involves the endocytic pathway. J. Biol. Chem. 269:17386-17389, 1994.

Koo E H, Sisodia S S, Archer D A, Martin U, Weidemann A, Beyreuther K, Masters C L, Fischer P, Price D L. Precursor of amyloid protein in Alzheimer's disease undergoes fast anterograde axonal transport. Proc. Natl. Acad. Sci. USA. 87:1561-1565, 1990.

Kounnas M Z, Moir R D, Rebeck G W, Bush A I, Agraves W S, Tanzi R E, Hyman B T, Strickland D K. LDL receptor-related protein, a multifunctional apoE receptor, binds secreted β-amyloid precursor protein and mediates its degradation. Cell. 82:331-340, 1995.

Krieger M and Herz J. Structures and functions of multiligand lipoprotein receptors: Macrophage scavenger receptors and LDL receptor-related protein (LRP). Ann. Rev. Biochem. 63:601-637, 1994.

Lamb B T, Bardel K A, Kulnane L S, Anderson J, Holtz G, Wagner S L, Sisodia S, Hoeger E J. Amyloid production and deposition in mutant amyloid precursor protein and presenilin-1 yeast artificial chromosome transgenic mice. Nature Neuroscience 2:695-697, 1999.

Lammich S, Kojro E, Postina R, Gilbert S, Pfeiffer R, Jasionowski M, Haass C, Fahrenholz F. Constitutive and regulated α-secretase cleavage of Alzheimer's amyloid precursor protein by a disintegrin metalloprotease. Proc. Natl. Acad. Sci U.S.A. 96:3922-3927,1999.

Lau K F, McLoughlin D M, Standen C L, Irving N G, Miller C C. Fe65 and X11b co-localize with and compete for binding to the amyloid precursor protein. NeuroReport. 11:3607-3610, 2000.

LeBlanc A C and Gambetti P. Production of Alzheimer 4 kDa β-amyloid peptide requires the C-terminal cytosolic domain of the amyloid precursor protein. Biochem. Biophys. Res. Commun. 204:1371-1380, 1994.

Lee S -J, Liyanage U, Bickel P E, Xia W, Lansbury P T, Kosik K. A detergent-insoluble membrane compartment contains Aβ in vivo. Nature Medicine. 4:730-734,1999.

Li Y, Marzolo M P, van Kerkhof P, Strous G J, Bu G. The YXXL motif, but not the two NPXY motifs, serves as the dominant endocytosis signal for low density lipoprotein receptor-related protein. J. Biol. Chem. 275:17187-17194, 2000.

Li Y M, Lai M; Xu M, Huang Q, DiMuzio-Mower J, Sardana M K, Shi X P, Yin K C, Shafer J A, Gardell S J. Presenilin 1 is linked with □-secretase activity in the detergent solubilized state. Proc. Natl. Acad. Sci U.S.A. 97:6138-6143, 2000.

Loukinova, E., S. Ranganathan, S. Kuznetsov, N. Gorlatov, M. M. Migliorini, D. Loukinov, P. G. Ulery, I. Mikhailenko, D. A. Lawrence, and D. K. Strickland. (2002) PDGF-induced tyrosine phosphorylation of the LDL receptor-related protein (LRP): Evidence for integrated co-receptor function between LRP and the PDGF receptor. J. Biol. Chem. 19:19.

Maltese W A, Wilson S, Tan Y, Suomensaari S, Sinha S, Barbour R, McConlogue L. Retention of the Alzheimer's amyloid precursor fragment C99 in the endoplasmic reticulum prevents formation of amyloid β-peptide. J. Biol. Chem. 276:20267-20279, 2001.

Marquez-Sterling N R, Lo A C, Sisodia S S, Koo E H. Trafficking of cell-surface β amyloid precursor protein: evidence that a sorting intermediate participates in synaptic vesicle recycling. J. Neurosci. 17:140-151, 1997.

Matsuda S, Yasukawa T, Homma Y, Ito Y, Niikura T, Hiraki T, Hirai S, Ohno S, Kita Y, Kawasumi M, Kouyama K, Yamamoto T, Kyriakis J M, Nishimoto I. c-Jun N-terminal kinase (JNK)-interacting protein-1b/islet-brain-1 scaffolds Alzheimer's amyloid precursor protein with JNK. J. Neurosci. 21:6597-6607, 2001.

May, P., Y. K. Reddy, and J. Herz. (2002) Proteolytic processing of LRP mediates regulated release of its intracellular domain. J. Biol. Chem. 20:20.

Mayford M, Bach M E, Huang Y -Y, Wang W, Hawkins R D, Kandel E R. Controlling memory formation through regulated expression of a CAMKII transgene. Science. 274:1678-1683, 1996.

Mayford M, Mansuy I M, Muller R U, Kandel E R. Memory and behavior: a second generation of genetically modified mice. Current Biology. 7:R580-R589,1997.

Mikhailenko I, Battey F D, Migliorini M, Ruiz J F, Argraves K, Moayeri M, Strickland D K. Recognition of α 2-macroglobulin by the low density lipoprotein receptor-related protein requires the cooperation of two ligand binding cluster regions. J. Biol. Chem. 276:39484-39491, 2001.

Moestrup K and Gliemann J. Analysis of Ligand Recognition by the Purified α-2-Macroglobulin Receptor (Low Density Lipoprotein Receptor-related Protein); Evidence that high affinity of α-2macroglobulin-protinease complex is achieved by binding to adjacent receptors. J. Biol. Chem. 266:14011-14017, 1991.

Morgenstern, J. P., and H. Land. (1990) Advanced mammalian gene transfer:

high titre retroviral vectors with multiple drug selection markers and a complementary helper-free packaging cell line. Nucleic Acid Res., 18, 3587-96.

Morishima-Kawashima M and Ihara Y. The presence of amyloid β-protein in the detergent-insoluble membrane compartment of human neuroblastoma cells. Biochemistry. 37:15247-15253, 11-3-1998.

Morris S M and Cooper J A. Disabled-2 colocalizes with the LDLR in clathrin-coated pits and interacts with AP-2. Traffic. 2:111-123,2001.

Murayama O, Tomita T, Nihonmatsu N, Murayama M, Sun X, Honda T, Iwatsubo T, Takashima A. Enhancement of amyloid β42 secretion by 28 different presenilin 1 mutations of familial Alzheimer's disease. Neurosci. Lett. 265:61-63, 1999.

Narita M, Holtzman D M, Schwartz A L, Bu G. α-2-macroglobulin complexes with and mediates the endocytosis of β-amyloid peptide via cell surface low-density lipoprotein receptor-related protein. J. Neurochem. 69:1904-1911,1997.

Nishimoto I, Okamoto K, Matsuura Y, Takahashi S, Murayama Y, Ogata E. Alzheimer amyloid protein precursor complexes with brain GTP-binding protein G0. Nature. 362:75-79, 1993.

Parkin E T, Hussain I, Turner A J, Hooper N M. Characterization of detergent-insoluble complexes containing the familial Alzheimer's Disease-associated presenilins. J. Neurochem. 1534-1543, 1999.

Parkin E T, Turner A J, Hooper N M. Amyloid precursor protein, although partially detergent-insoluble in mouse cerebral cortex, behaves as an atypical lipid raft protein. Biochem. J. 344 Pt 1:23-30, 1999.

Perez R G, Soriano S, Hayes J D, Ostaszewski B, Xia W, Selkoe D J, Chen X, Stokin G B, Koo E H. Mutagenesis identifies new signals for β-amyloid precursor protein endocytosis, turnover, and the generation of secreted fragments, including Aβ42. J. Biol. Chem. 274:18851-18856, 1999.

Perez R G, Squazzo S L, Koo E H. Enhanced release of the amyloid β-protein from codon 670/671 “Swedish” mutant β-amyloid precursor protein occurs in both secretory and endocytic pathways. J. Biol. Chem. 271:9100-9107, 1996.

Perez R G, Zheng H, Van der Ploeg L H T, Koo E H. The β-amyloid procursor protein of Alzheimer's disease enhances neuron viability and modulates neuronal polarity. J. Neurosci. 17:9407-9414, 1997.

Prasher V P, Farrer M J, Kessling A M, Fisher E M C, West R J, Barber P C, Butler A C. Molecular mapping of Alzheimer-type dementia in Down's syndrome. Ann. Neurol. 43:380-383, 1998.

Rebeck G W, Moir R D, Mui S, Strickland D K, Tanzi R E, Hyman B T. Association of membrane-bound amyloid precursor protein APP with the apolipoprotein E receptor LRP. Brain Res. Mol. Brain Res. 87:238-245,2001.

Riddell D R, Christie G, Hussain I, Dingwall C. Compartmentalization of β secretase (Asp2) into low-buoyant density, noncaveolar lipid rafts. Current Biology. 11:1288-1293,2001.

Rockenstein E, Mallory M, Mante M, Sisk A, Masliah E. Early formation of mature amyloid-beta protein deposits in a mutant APP transgenic model depends on levels of Abeta(1-42). J. Neurosci. Res. 66:573-582, 2001.

Russo T, Faraonio R, Minopoli G, De Candia P, De Renzis S, Zambrano N. Fe65 and the protein network centered around the cytosolic domain of the Alzheimer's β-amyloid precursor protein. FEBS Lett. 434:1-7, 1998.

Sabo S, Lanier L M, Ikin A F, Khorkova O, Sahasrabudhe S R, Greengard P, Buxbaum J D. Regulation of b-amyloid secretion by FE65, an anyloid protein precursor-binding protein. J. Biol. Chem. 274,No. 12:7952-7957, 1999.

Sastre M, Turner R S, Levy E. X11 interaction with β amyloid precursor protein modulates its cellular stabilization and reduces amyloid b-protein secretion. J. Biol. Chem. 273,No35:22351-22357, 1998.

Scheinfeld M H, Roncarati R, Vito P, Lopez P A, Abdallah M, D'Adamio L. Jun NH2-terminal kinase (JNK) interacting protein 1 (JIP1) binds the cytoplasmic domain of the Alzheimer's beta-amyloid precursor protein (APP). J. Biol. Chem. 277:3767-3775, 2002.

Schwarze S R, Ho A, Vocero-Akbani A, Dowdy S F. In vivo protein transduction: delivery of a biologically active protein into the mouse. Science. 285:1569-1572, 1999.

Schwarze S R, Hruska K A, Dowdy S F. Protein transduction: unrestricted delivery into all cells? Trends Cell Biol. 10:290-295, 2000.

Selkoe D J. Alzheimer's disease: genes, proteins, and therapy. Physiol Rev. 81:741-766,2001.

Shibata M, Yamada S, Kumar S R, Calero M, Bading J, Frangione B, Holtzman D M, Miller C A, Strickland D K, Ghiso J, Zlokovic B V. Clearance of Alzheimer's amyloid-β₁₋₄₀ peptide from brain by LDL receptor-related protein-1 at the blood-brain barrier [In Process Citation]. J. Clin. Invest. 106:1489-1499, 2000.

Simons M, Keller P, De Strooper B, Beyreuther K, Dotti C G, Simons K. Cholesterol depletion inhibits the generation of β-amyloid in hippocampal neurons. Proc. Natl. Acad. Sci. USA. 95:6460-6464,1999.

Simons M, Keller P, Dichgans J, Schulz J B. Cholesterol and Alzheimer's disease: is there a link? Neurology. 57:1089-1093, 2001.

Sinha S, Anderson J P, Barbour R, Basi G S, Caccavello R, Davis D, Doan M, Dovey H F, Frigon N, Hong J, Jacobson-Croak K, Jewett N, Keim P, Knops J, Lieberburg I, Power M, Tan H, Tatsuno G, Tung J, Schenk D, Seubert P, Suomensaari S M, Wang S, Walker D, John V,. Purification and cloning of amyloid precursor protein β-secretase from human brain. Nature. 402:537-540, 1999.

Sisodia S S, Koo E H, Beyreuther K, Unterbeck A, Price D L. Evidence that beta-amyloid protein in Alzheimer's disease is not derived by normal processing. Science. 248:492-495, 1990.

Sisodia S S, Koo E H, Hoffman P N, Perry G, Price D L. Identification and transport of full-length amyloid precursor proteins in rat peripheral nervous system. J. Neurosci. 13:3136-3142, 1993.

Skovronsky D M, Doms R W, Lee V M Y. Detection of a novel intraneuronal pool of insoluble amyloid β protein that accumulates with time in culture. 141:1031-1039, 1999.

Strickland D K, Ashcom J D, Williams S, Battey F, Behre E, McTigue K, Battey J F, Argraves W S. Primary structure of α2-macroglobulin receptor-associated protein. Human homologue of a Heymann nephritis antigen. J. Biol. Chem. 266:13364-13369, 1991.

Teplow D B. Structure and kinetic features of amyloid β-protein fibrillogenesis. Amyloid. 5:121-142, 2002.

Thinakaran G, Teplow D B, Siman R, Greenberg B, Sisodia S S. Metabolism of the “Swedish” amyloid precursor protein variant in neuro2a (N2a) cells: Evidence that cleavage at the “β-secretase” site occurs in the golgi appartus. J. Biol. Chem. 271:9390-9397, 1996.

Tienari P J, Ida N, Ikonen E, Simons M, Weidemann A, Multhaup G, Masters C L, Dotti C G, Beyreuther K. Intracellular and secreted Alzheimer β-amyloid species are generated by distinct mechanisms in cultured hippocampal neurons. Proc. Natl. Acad. Sci U.S.A. 94:4125-4130, 1997.

Tomlinson B E. The neuropathology of Alzheimer's disease. Neuropath. Appl. Neurobiol. 15: 491-512, 1989.

Trommsdorff, M., J. P. Borg, B. Margolis, and J. Herz. (1998) Interaction of cytosolic adaptor proteins with neuronal apolipoprotein E receptors and the amyloid precursor protein. J. Biol. Chem., 273, 33556-60.

Ulery P G, Beers J, Mikhailenko I, Tanzi R E, Rebeck G W, Hyman B T, Strickland D K. Modulation of β-amyloid precursor protein processing by the low density lipoprotein receptor-related protein (LRP). Evidence that LRP contributes to the pathogenesis of Alzheimer's disease. J. Biol. Chem. 275:7410-7415, 2000.

Van Gassen G, Annaert W, Van Broeckhoven C. Binding Partners of Alzheimer's Disease Proteins: Are They Physiologically Relevant? Neurobiol. Dis. 7:135-151, 2000.

Vassar R, Bennett B D, Babu-Khan S, Kahn S, Mendiaz E A, Denis P, Teplow D B, Ross S, Amarante P, Loeloff R, Luo Y, Fisher S, Fuller J, Edenson S, Lile J, Jarosinski M A, Biere A L, Curran E, Burgess T, Louis J C, Collins F, Treanor J, Rogers G, Citron M. □-secretase cleavage of Alzheimer's amyloid precursor protein by the transmembrane aspartic protease BACE. Science. 286:735-741, 1999.

Wadia J S and Dowdy S F. Protein transduction technology. Curr. Opin. Biotechnol. 13:52-56, 2002.

Ward, D. M., R. Ajioka, and J. Kaplan. (1989) Cohort movement of different ligands and receptors in the intracellular endocytic pathway of alveolar macrophages. J. Biol. Chem., 264, 8164-70.

Weggen S, Eriksen J L, Das P, Sagi S A, Wang R, Pietrzik C U, Findlay K A, Smith T E, Murphy M P, Bulter T, Kang D E, Marquez-Sterling N, Golde T E, Koo E H. A subset of NSAIDs lower amyloidogenic Aβ42 independently of cyclooxygenase activity. Nature. 414:212-216, 2001.

Wild-Bode C, Yamazaki T, Capell A, Leimer U, Steiner H, Ihara Y, Haass C. Intracellular generation and accumulation of amyloid β-peptide terminating at amino acid 42. J. Biol. Chem. 272:16085-16088, 1997.

Willnow T E, Orth K, Herz J. Molecular dissection of ligand binding sites on the low density lipoprotein receptor-related protein. J. Biol. Chem. 269:15827-15832, 1994.

Willnow T E, Rohlmann A, Horton J, Otani H, Braun J R, Hammer R E, Herz J. RAP, a specialized chaperone, prevents ligand-induced ER retention and degradation of LDL receptor-related endocytic receptors. EMBO J. 15:2632-2639, 1996.

Wolfe M S, Welming X, Ostaszewski B L, Diehl T S, Kimberly W T, Selkoe D J. Two transmembrane aspartates in presenillin-1 required for presenilin endoproteolysis and γ-secretase activity. Nature. 398:513-517, 1999.

Wolozin B. A fluid connection: cholesterol and Aβ. Proc. Natl. Acad. Sci U.S.A. 98:5371-5373,2001.

Xia W, Zhang J, Perez R, Koo E H, Selkoe D J. Interaction between amyloid precursor protein and presenilins in mammalian cells: Implications for the pathogenesis of Alzheimer disease. Proc. Natl. Acad. Sci. USA. 94:8208-8213, 1997.

Xia X, Qian S, Soriano S, Wu Y, Fletcher A M, Wang X J, Koo E H, Wu X, Zheng H. Loss of presenilin 1 is associated with enhanced β-catenin signaling and skin tumorigenesis. Proc. Natl. Acad. Sci U.S.A. 98:10863-10868, 2001.

Yamamoto A, Lucas J J, Hen R. Reversal of neuropathology and motor dysfunction in a conditional model of Huntington's disease. Cell. 101:57-66,2000.

Yamazaki T, Koo E H, Selkoe D J. Trafficking of cell surface amyloid β-protein precursor. II. Endocytosis, recycling, and lysosomal targeting detected by immunolocalization. J. Cell Science. 109:999-1008, 1996.

Yan R, Han P, Miao H, Greengard P, Xu H. The transmembrane domain of the Alzheimer's β-secretase (BACE1) determines its late Golgi localization and access to β-amyloid precursor protein (APP) substrate. J. Biol. Chem. 276:36788-36796, 2001.

Yankner B A. Mechanisms of neuronal degeneration in Alzheimer's disease. Neuron. 16:921-932, 1996.

Yu G, Nishimura M, Arawaka S, Levitan D, Zhang L, Tandon A, Song Y Q, Rogaeva E, Chen F, Kawarai T, Supala A, Levesque L, Yu H, Yang D S, Holmes E, Milman P, Liang Y, Zhang D M, Xu D H, Sato C, Rogaev E, Smith M, Janus C, Zhang Y, Aebersold R, Farrer L S, Sorbi S, Bruni A, Fraser P, George-Hyslop P. Nicastrin modulates presenilin-mediated notch/glp-1 signal transduction and EOAPP processing. Nature. 407:48-54, 2000.

Zheng P, Eastman J, Vande P S, Pimplikar S W. PAT 1, a microtubule-interacting protein, recognizes the basolateral sorting signal of amyloid precursor protein. Proc. Natl. Acad. Sci U.S.A. 95:14745-14750, 1998.

What is claimed is:

1. A truncated dominant negative mammalian LDL receptor related protein (LRP) cytoplasmic tail mutant (LRP-CT) molecule, comprising:
a signal peptide; and
a peptide sequence of about 100 to 400 amino acids at the cytoplasmic tail end of LRP , encompassing the entire transmembrane and cytoplasmic domains, and having a deletion of 22 amino acids, from residue number 4486 to residue number 4507, from an LRP molecule having 4544 amino acid residues, the deletion encompassing the domain that is critical in processing amyloid precursor protein (APP).

2. A DNA sequence for constructs for expressing truncated LDL receptor related protein (LRP) cytoplasmic tail mutant (LRP-CT) molecule selected from the group consisting of SEQ ID NO 1, SEQ ID NO 3, SEQ ID NO 4, SEQ ID NO 5, SEQ ID NO 6, SEQ ID NO 7, SEQ ID NO 8, SEQ ID NO 9, SEQ ID NO 10, SEQ ID NO 11, and SEQ ID NO 12.

3. A method for disrupting generation of amyloid β-protein (Aβ), comprising:
constructing a truncated LDL receptor related protein cytoplasmic tail mutant (LRP-CT);
introducing and overexpressing the mutant in a normal mammalian cell; and
allowing the mutant to react with an amyloid precursor protein (APP), required for producing Aβ, produced by the cell
wherein the reacting of the LRP-CT mutant protein with the APP reactive site competes with LRP diminishing formation of an APP:LRP complex and thereby interferes with enzymatic processing of APP that is required in Aβ production.

4. The method according to claim 3, wherein the interaction requires a third, adaptor, molecule.

5. The method according to claim 4, wherein the adaptor molecule is Fe65.

6. The method according to claim 3, wherein the inhibition is effectuated by a functional analogue of the truncated LRP-CT molecule.

7. The method according to claim 6, wherein the analogue of the truncated LRD-CT molecule is selected from the group comprising reagents, drugs, compounds peptides, peptidomimetics and antibodies to the reactive site of either APP or LRP cytoplasmic tail region.

8. A method for preventing or treating diseases wherein amyloid β-protein (Aβ) is a major constituent of amyloid plaques or amyloidosis by interfering with production of Aβ, comprising:
contacting the affected cells or tissues of a recipient with a truncated dominant negative LDL receptor related protein cytoplasmic tail mutant (LRP-CT) consisting of the signal peptide and 100-400 amino acids of cytoplasmic tail of LRP, encompassing the entire transmembrane and cytoplasmic domains, and having a deletion of 22 amino acids, from residue number 4486 to residue number 4507, from an LRP molecule having 4544 amino acid residues, the deletion encompassing the putative domain that is critical in processing amyloid precursor protein (APP), or an analogue thereof;
permitting the LRP-CT construct to internalize into the target cells;
allowing the construct to interact with APP or an adaptor molecule wherein the interaction results in binding to the APP or adaptor molecule, and wherein the binding competes with wild-type APP and interferes with enzymic processing of the APP into Aβ,
thereby preventing the triggering of a cascade of molecular events causing amyloid plaque formation found in these diseases; and
monitoring the recipient's Aβ profile.

9. The method according to claim 8, wherein diminished production of Aβ interferes with the cascade that results in development of Alzheimer's Disease.

10. A high throughput assay for screening compounds that inhibit AP production, comprising:
combining in an appropriate reaction chamber,
a substrate molecule;
a test molecule; and
a reporter molecule;
permitting the reaction to proceed for an appropriate time; and
detecting the loss of binding capability by the reporter molecules.
wherein the loss of binding represents interference and loss of the LRP (or APP):Fe65 interaction due to competition by the candidate test molecule.

11. The method according to claim 10, wherein the loss of interaction is between APP and Fe65,

12. The method according to claim 11, wherein the loss of interaction is between LRP and Fe65.

13. The method according to claim 10, wherein the reporter molecule is labeled.

14. The method according to claim 13, wherein the label is selected from a group consisting essentially of fluorescent dyes, enzyme reaction products, radioisotopes or

15. A method for inhibiting Aβ production by impairing formation of an LRP-Fe65-APP trimeric complex by blocking active receptor sites of any of the three components capable of forming the complex.

16. The method according to claim 15, wherein the impairing is by a truncated cytoplasmic tail of LRP.

17. The method according to claim 16, wherein the impairing is by the truncated cytoplasmic tail of LRP having sequences SEQ ID NOs 1, 2-8, and their analogs or functional equivalents.

18. The method according to claim 17, wherein the impairing is by a small functionally analogous molecule selected from the group consisting essentially of peptides, peptidomimetics, drugs or antibodies to the active site.

19. A method for inhibiting LRP or APP:Fe65 interaction in vivo, comprising:
administering to a recipient an effective amount of inhibitor compound to prevent the formation of LRP-Fe65-APP trimeric complexes facilitating catabolism of APP to produce Aβ.

20. The method according to claim 19, wherein the administering is orally.

21. The method according to claim 19, wherein the administering is by taking an effective drug in liquid or tablet form.

22. The method according to claim 19, wherein the administering is by injection intravenously or intraperitoneally, intracranially.

23. The method according to claim 19, wherein the administering is by infusion intravenously.

24. The method according to claim 19, wherein the administering is by gene therapy.

25. The method according to claim 19, wherein the administering is implanting into a recipient a time-release substance.

26. The method according to claim 19, wherein the administering is to the brain is facilitated by designing molecules that permeate the blood/brain barrier.

27. A kit suitable for providing the required reactants for screening assays, comprising:
a vessel of appropriate size containing APP;
a vessel containing an appropriate amount of Fe65 adaptor peptide;
a signal substance; and
instructions, including an appropriate standardized profile chart.
